(19) United States
(12) Patent Application Publication
(10) Pub. No.: US 2026/0000374 A1
(43) Pub. Date: Jan. 1, 2026

(54) CROSS GRID PHASE CONTRAST X-RAY COMPUTED TOMOGRAPHY

(71) Applicant: Battelle Memorial Institute, Richland, WA (US)

(72) Inventors: Erin A. Miller, Richland, WA (US); Andrew J. Gilbert, Richland, WA (US); Timothy A. White, Richland, WA (US); Nikhil S. Deshmukh, Richland, WA (US); Luke W. Campbell, Richland, WA (US); Dustin M. Kasparek, Richland, WA (US)

(73) Assignee: Battelle Memorial Institute, Richland, WA (US)

(21) Appl. No.: 19/320,969

(22) Filed: Sep. 5, 2025

Related U.S. Application Data
(63) Continuation-in-part of application No. 18/655,160, filed on May 3, 2024.
Application No. 18/655,160 is a continuation-in-part of application No. 18/519,861, filed on Nov. 27, 2023, now abandoned.
Application No. 18/519,861 is a continuation of application No. 17/442,340, filed on Sep. 23, 2021, now Pat. No. 11,826,187.
Application No. 17/442,340 is a continuation of application No. 16/363,989, filed on Mar. 25, 2019, now Pat. No. 11,006,912.

(51) Int. Cl. A61B 6/42; A61B 6/00; A61B 6/40; A61B 6/46

(52) U.S. Cl. CPC A61B 6/4291; A61B 6/4035; A61B 6/405; A61B 6/466; A61B 6/484

(57) ABSTRACT

Systems include an x-ray optical arrangement that includes an x-ray source configured to emit an x-ray beam along a beam path and through an object placement location situated to receive an object for inspection, an object grating situated along the beam path, and a detector situated along the beam path to receive the x-ray beam after propagating through the object grating and object placement location and to detect image data, wherein the object grating includes object grating elements arranged in an object grating pattern; and a movement stage providing a plurality of computed tomography imaging positions for the object by the x-ray optical arrangement; wherein the object grating pattern and a positioning of the x-ray source, object grating, and detector in relation to each other are configured to provide a plurality of computed tomography detection modes in the image data.

### CROSS REFERENCE TO RELATED APPLICATIONS

This is a continuation-in-part of U.S. patent application Ser. No. 18/655,160, filed May 3, 2024, which is a continuation-in-part of U.S. patent application Ser. No. 18/519,861, filed Nov. 27, 2023, which is a continuation of U.S. patent application Ser. No. 17/442,340, filed Sep. 23, 2021, now issued as U.S. Pat. No. 11,826,187, which is a U.S. National Stage of International Application No. PCT/US2020/023884, filed Mar. 20, 2020, which was published in English under PCT Article 21(2), which is a continuation of U.S. patent application Ser. No. 16/363,989, filed Mar. 25, 2019, now issued as U.S. Pat. No. 11,006,912, all of which are incorporated herein in their entirety.

### ACKNOWLEDGEMENT OF GOVERNMENT SUPPORT

This invention was made with Government support under Contract DE-AC0576RL01830 awarded by the U.S. Department of Energy. The Government has certain rights in the invention.

### FIELD

The present disclosure relates to phase-contrast x-ray imaging, and more particularly to phase-contrast x-ray imaging having improved quality and material discriminating power, and even more particularly with phase-contrast x-ray imaging used to provide computer tomography image data.

### BACKGROUND

X-ray based imaging is used in a variety of non-destructive examination (NDE) applications. In many of these applications, which can range from medical imaging to security screening, the primary x-ray characteristics are density and effective atomic number derived from multispectral x-ray attenuation measurements. The addition of phase contrast as a third imaging characteristic can improve material discrimination by detection of refractive and scattering effects in examined objects. However, unwanted spectral effects, misleading image artifacts, and the demands associated with producing images with increased material penetration required novel solutions in order to improve resultant x-ray images. Such solutions are described herein.

### SUMMARY

Disclosed are methods, systems, and non-transitory, computer-readable storage media storing programs for phase-contrast x-ray imaging having improved quality and material discriminating power.

In some embodiments, a method comprises emitting source x-rays from a polychromatic source operating at an endpoint energy greater than or equal to 100 keV and generating a spot size greater than or equal to 0.5 mm; creating a series of periodically repeating apparent sources from the source x-rays using a source grating; patterning the series of periodically repeating apparent sources into a patterned beam using an object grating placed proximal to an object to be imaged and at distances L₁ from the source grating and L₂ from a detector grating, wherein the periodicities, P, of the source and object grating elements are related by P_{source}=P_{object}*[(L₁+L₂)/L₂] and wherein the source and object grating elements are substantially parallel; acquiring through the detector grating a first image with the object and a second image without the object, wherein the detector grating is oriented substantially orthogonally relative to the object grating and beam axis and wherein the object grating and the detector grating have a substantially equivalent x-ray attenuating factor; measuring visibilities of the object grating from the first and second images to determine an object grating visibility reduction due to scatter and beam hardening; measuring visibilities of the detector grating from the first and second images to determine a detector grating visibility reduction due to beam hardening; and applying a beam hardening correction based on a comparison of the object grating visibility reduction and the detector grating visibility reduction to generate a corrected scatter image.

In certain embodiments, the method can further comprise operating the polychromatic source at an endpoint energy greater than or equal to 150 keV, 160 keV, 175 keV, 200 keV, or 450 keV. In certain embodiments, the method can further comprise tilting the object grating and detector grating by rotating the gratings about an axis parallel to grating element lines. In certain embodiments, the method can further comprise tilting the source grating by rotating the gratings about an axis parallel to grating element lines.

In certain embodiments, the object grating is approximately equidistant between the source and the detector. In certain embodiments, the detector grating has a periodicity, P_{detector}, equivalent to that of the source grating, P_{source}. In certain embodiments, the object and detector gratings comprise an equivalent material and have an equivalent thickness. In certain embodiments, the source grating, object grating, detector grating, or combinations thereof have grating elements comprising a parallel line pattern.

In certain embodiments, the object to be imaged is a scatter test object calibration standard and further comprising performing a calibration of x-ray scatter, the scatter test object calibration standard comprising metal or metal oxide particles distributed in a polymer matrix and having a stepped-wedge geometry of at least three different thicknesses. In certain embodiments, the object to be imaged is a beam hardening test object calibration standard and further comprising performing a calibration of beam hardening, the beam hardening test object calibration standard comprising three or more homogeneous materials in a range of atomic numbers, with no large density variations on length scales between 10 nm and 200 microns, and have a thickness such that 10-90% of the x-ray intensity is transmitted through the test object.

In some embodiments, a system comprises a polychromatic x-ray source configured to provide source x-rays at an endpoint energy greater than or equal to 100 keV and a spot size greater than 0.5 mm; a source grating configured to create a series of periodically repeating apparent sources from the source x-ray; an object grating proximal to a position of an object to be imaged and at distances L₁ from the source grating and L₂ from a detector grating, wherein the periodicities of the source and object gratings are related by P_{source}=P_{object}*[(L₁+L₂)/L₂], the object grating configured to pattern the series of periodically repeating apparent sources into a patterned beam; and a detector grating having detector grating elements that are oriented orthogonally relative to object grating elements and a beam axis, the detector and object gratings having an equivalent x-ray attenuation factor. The system further comprises processing circuitry operably connected to the detector and configured to execute computer-readable instructions to acquire through the detector grating a first image with the object and a second image without the object; measure visibilities of the object grating from the first and second images to determine an object grating visibility reduction due to scatter and beam hardening; measure visibilities of the detector grating from the first and second images to determine a detector grating visibility reduction due to beam hardening; and apply a beam hardening correction based on a comparison of the object grating visibility reduction and the detector grating visibility reduction to generate a corrected scatter image.

In certain embodiments, the polychromatic source is configured to provide source x-rays at an endpoint energy greater than or equal to 150 keV, 160 keV, 175 keV, 200 keV, or 450 keV. In certain embodiments, the object grating and detector grating are positioned such that object grating elements and detector grating elements are tilted by a rotation of the gratings about an axis parallel to grating element lines. In certain embodiments, the source grating is positioned such that source grating elements are tilted by a rotation of the gratings about an axis parallel to grating element lines. In certain embodiments, the object grating is positioned approximately equidistant between the source and the detector. In certain embodiments, the detector grating has a periodicity, P_{detector}, equivalent to that of the source grating, P_{source}. In certain embodiments, the detector grating abuts the detector. In certain embodiments, the object and detector gratings comprise an equivalent material and have an equivalent thickness. In certain embodiments, the source grating, object grating, detector grating, or combinations thereof have grating elements comprising a parallel line pattern.

In some embodiments, a non-transitory computer readable storage medium stores one or more programs, the one or more programs comprise instructions, which when executed by one or more processors operably connected to an x-ray imaging system, cause the system to acquire through the detector grating a first image with the object and a second image without the object; measure visibilities of the object grating from the first and second images to determine an object grating visibility reduction due to scatter and beam hardening; measure visibilities of the detector grating from the first and second images to determine a detector grating visibility reduction due to beam hardening; and apply a beam hardening correction based on a comparison of the object grating visibility reduction and the detector grating visibility reduction to generate a corrected scatter image. The x-ray imaging system to which the processor(s) are operably connected comprise a polychromatic x-ray source configured to provide source x-rays at an endpoint energy greater than or equal to 100 keV and a spot size greater than 0.5 mm; a source grating configured to create a series of periodically repeating apparent sources from the source x-ray; an object grating proximal to a position of an object to be imaged band at distances L₁ from the source grating and L₂ from a detector grating, wherein the periodicities of the source and object gratings are related by P_{source}=P_{object}*[(L₁+L₂)/L₂], the object grating configured to pattern the series of periodically repeating apparent sources into a patterned beam; and a detector grating having detector grating elements that are oriented orthogonally relative to object grating elements and a beam axis, the detector and object gratings having an equivalent x-ray attenuation factor.

In certain embodiments, the non-transitory computer readable storage medium storing one or more programs, the one or more programs comprising instructions, which when executed by one or more processors operably connected to the x-ray imaging system further cause the x-ray imaging system to perform a calibration, wherein the object to be imaged is a scatter test object, a beam hardening test object, or both.

According to an aspect of the disclosed technology, systems include an x-ray source configured to emit an x-ray beam along a beam path and through an object arranged for inspection in a field of view of the x-ray source; and an object grating, an analyzer grating, a detector grating, and a detector arranged with respect to each other in the field of view; wherein the object grating includes object grating elements arranged in a first pattern, the detector grating includes detector grating elements arranged in a second pattern that is separable from the first pattern, and the analyzer grating includes analyzer grating elements that are arranged to correspond to a combination of the first pattern and second pattern; wherein the analyzer grating, and/or the object grating and detector grating, are configured to move relative to each other to different phase positions, and wherein the detector is configured to collect indirect moiré image data of the object at the different phase positions. In some examples, the wherein the analyzer grating, and/or the object grating and detector grating, are configured to move relative to each other to the different phase positions by translating the analyzer grating transverse to the beam path to the different phase positions while the detector grating and object grating are fixed. In some examples, the object grating and detector grating are configured to move relative to the analyzer grating to the different phase positions by translating the object grating and detector grating transverse to the beam path to the different phase positions while the analyzer grating is fixed. In some examples, the object grating elements include a set of parallel linear object grating elements, the detector grating elements include a set of parallel linear detector grating elements arranged perpendicularly with respect to the parallel linear object grating elements, and the analyzer grating elements include a set of crossed grating elements. Some examples further include one or more movement stages coupled to the analyzer grating and/or the object grating and detector grating to provide the relative movement to the different phase positions. In some examples, the relative movement to different phase positions includes a first set of positions along a first axis aligned with a direction of the first pattern and a second set of positions along a second axis aligned with a direction of the second pattern. In some examples, the relative movement to different phase positions further includes a set of one or more additional off-axis positions configured to average spectral information associated with the object grating and detector grating and thereby reduce moiré artifacts associated with the moiré image data. In some examples, the relative movement to different phase positions further includes a set of positions to define a latticed array of positions. In some examples, the relative movement to the different phase positions includes a third set of positions aligned with the direction of the first pattern and spaced apart from the first set of positions, and a fourth set of positions aligned with the direction of the second pattern and spaced apart from the second set of positions. In some examples, the third set of positions is spaced apart from the first set of positions by a pi-shift and the fourth set of positions is spaced apart from the second set of positions by a pi-shift. Some examples further include a processor and memory configured with processor executable instructions which cause the processor to apply a beam hardening correction to the indirect moiré image data. Some examples further include a source grating situated adjacent to the x-ray source and configured to receive the x-rays from the x-ray source to produce a plurality of source grating x-ray sources. In some examples, the object grating, analyzer grating, and detector gratings are arranged in the field of view along the beam path such that the analyzer grating precedes the detector, the detector grating precedes the analyzer grating, and the position of the object to be inspected precedes the analyzer grating. In some examples, the object grating precedes the position of the object to be inspected.

According to another aspect of the disclosed technology, methods include emitting an x-ray beam from an x-ray source along a beam path and through a position for an object arranged to be inspected in a field of view of the x-ray source, wherein an object grating, an analyzer grating, a detector grating, and a detector are arranged with respect to each other in the field of view, wherein the object grating includes object grating elements arranged in a first pattern, the detector grating includes detector grating elements arranged in a second pattern that is separable from the first pattern, and the analyzer grating includes analyzer grating elements that are arranged to correspond to a combination of the first pattern and second pattern; moving the analyzer grating, and/or the object grating and detector grating, relative to each other to different phase positions; and detecting indirect moiré image data with the detector at the different phase positions. In some examples, the moving comprises translating the analyzer grating transverse to the beam path to the different phase positions while the detector grating and object grating are fixed. In some examples, the moving comprises translating the object grating and detector grating transverse to the beam path to the different phase positions while the analyzer grating is fixed. In some examples, the object grating elements include a set of parallel linear object grating elements, the detector grating elements include a set of parallel linear detector grating elements arranged perpendicularly with respect to the parallel linear object grating elements, and the analyzer grating elements include a set of crossed grating elements. In some examples, the moving comprises moving the analyzer grating, and/or the object grating and detector grating, relative to each other to the different phase positions with one or more movement stages. In some examples, the moving to the different phase positions comprises moving to a first set of positions along a first axis aligned with a direction of the first pattern and moving to a second set of positions along a second axis aligned with a direction of the second pattern. In some examples, the moving to the different phase positions further comprises moving to a third set of positions aligned with the direction of the first pattern and spaced apart from the first set of positions, and to a fourth set of positions aligned with the direction of the second pattern and spaced apart from the second set of positions. In some examples, the third set of positions is spaced apart from the first set of positions by a pi-shift and the fourth axis is spaced apart from the second set of positions by a pi-shift. In some examples, the moving to the different phase positions further includes moving to a set of one or more additional off-axis positions configured to average spectral information associated with the object grating and detector grating and thereby reduce moiré artifacts associated with the moiré image data. In some examples, the moving to the one or more additional off-axis positions includes moving to a set of positions such that the different phase positions comprise a crossed or L-shaped set of positions and a diagonal set of positions. In some examples, the moving to the one or more additional off-axis positions includes moving to a set of positions such that the different phase positions comprise a latticed array of positions. Some examples further include, with a processor and memory configured with processor executable instructions, applying a beam hardening correction to the indirect moiré image data. In some examples, the object grating, analyzer grating, and detector gratings are arranged in the field of view along the beam path such that the analyzer grating precedes the detector, the detector grating precedes the analyzer grating, and the position of the object to be inspected precedes the analyzer grating. In some examples, the object grating precedes the position of the object to be inspected.

According to another aspect of the disclosed technology, non-transitory computer readable storage media are provided, storing one or more programs, the one or more programs comprising instructions, which when executed by one or more processors operably connected to an x-ray imaging system that comprises: an x-ray source configured to emit an x-ray beam along a beam path and through an object arranged for inspection in a field of view of the x-ray source; and an object grating, an analyzer grating, a detector grating, and a detector arranged with respect to each other in the field of view; wherein the object grating includes object grating elements arranged in a first pattern, the detector grating includes detector grating elements arranged in a second pattern that is separable from the first pattern, and the analyzer grating includes analyzer grating elements that are arranged to correspond to a combination of the first pattern and second pattern; wherein the analyzer grating, and/or the object grating and detector grating, are configured to move relative to each other to different phase positions, and wherein the detector is configured to collect indirect moiré image data of the object at the different phase positions; causes the x-ray imaging system to collect the indirect moiré image data at the different phase positions.

Also disclosed are methods, systems, and non-transitory, computer-readable storage media storing programs for gratings-based x-ray computed tomography having improved characteristics.

According to an aspect of the disclosed technology, a system includes an x-ray optical arrangement that includes an x-ray source configured to emit an x-ray beam along a beam path through at least a portion of an object placement location situated to receive an object for inspection, through an object grating situated along the beam path, and to a detector situated along the beam path to receive the x-ray beam after propagating through the object grating and object placement location and to detect image data, wherein the object grating includes object grating elements arranged in an object grating pattern; and at least one movement stage coupled to the object placement location and/or the x-ray optical arrangement and configured to provide rotational movement between the x-ray optical arrangement and the object placement location to provide a plurality of computed tomography imaging positions for the object by the x-ray optical arrangement; wherein the object grating pattern and a positioning of the x-ray source, object grating, and detector in relation to each other are configured to provide a plurality of computed tomography detection modes in the image data. In some examples, the plurality of detection modes includes at least two of absorption, scatter, or differential phase. In some examples, the object grating elements include parallel line elements. In some examples, the object grating is situated such that the parallel line elements of the object grating are parallel to a rotational axis of the movement stage. In some examples, the at least one movement stage also provides for translational motion along a line parallel to the rotational axis between the x-ray optical arrangement and the object placement location, wherein the X-ray beam is configured with a height along the rotational axis that is less than a height of the object placement location, and wherein an effective detection height of the detector is less than the height of the object placement location, and wherein a plurality of computed tomography imaging positions comprise position taken at a plurality of different translational positions of the at least one movement stage. In some examples, the effective detection height of the detector includes a sufficient number of pixels to effectively detect at least one entire fringe spacing. Some examples further include a detector grating situated closer to the detector than the object placement location and including detector grating elements arranged in a detector grating pattern, wherein the detector grating elements include parallel line elements and the detector grating is situated such that the parallel line elements of the detector grating are perpendicular to the parallel line elements of the object grating. In some examples, the detector grating pattern is configured in relation to the object grating pattern to allow for a beam hardening correction to the image data through a separation of pattern visibility loss due to scatter from pattern visibility loss due to spectral changes. Some examples further include a source grating situated adjacent to the x-ray source and including a source grating pattern configured to produce a plurality of line sources from the x-ray beam. In some examples, the source grating pattern includes a plurality of parallel line elements, and the source grating is situated such that the parallel line elements of the source grating are parallel to the parallel line elements of the object grating. In some examples, the object grating is situated farther from the detector than the object placement location, or, wherein the object grating is situated closer to the detector than the object placement location. In some examples, the object grating is situated less than 0.45L, 0.4L, 0.3L, or 0.2L distance from the x-ray source, where L is the distance between the x-ray source and the detector. Some examples further include a processor and memory configured with processor-executable instructions which cause the processor to apply a beam hardening correction to the image data through a separation of pattern visibility loss due to scatter from pattern visibility loss due to spectral changes. Some examples further include a processor and memory configured with processor executable instructions which cause the processor to perform a computed tomography reconstruction with the image data for one or more of the computed tomography detection modes. In some examples, the memory is configured with processor executable instructions which cause the processor to perform the computed tomography reconstruction with the image data for the differential phase computed tomography detection mode, including Fourier transforming sinograms of the image data, filtering the transformations using a signum filter, and backprojecting the filtered transformations through the object space to obtain a slice; wherein the object grating includes parallel line elements arranged parallel to a rotational axis of the movement stage. In some examples, the memory is configured with processor executable instructions which cause the processor to perform the computed tomography reconstruction with the image data for the differential phase computed tomography detection mode, including integrating differential phase image data, Fourier transforming sinograms of the differential phase image data, filtering the transformations using a ramp filter, and backprojecting the filtered transformations through the object space to obtain a slice. Some examples further include a processor and memory configured with processor executable instructions which cause the processor to: Fourier transform the detected image data of an object-free region from which pre- and post-exposure grating-only image data can be compared; and fit Fourier components of the transformed detected image data that are in a Fourier space region around a source grid oscillation peak, to a linear combination of Fourier components of the pre- and post-exposure grating-only image data so that the linear combination can be used as a reference grating image during image processing to extract the phase and scatter images.

According to another aspect of the disclosed technology, a method includes, with an x-ray optical arrangement having an x-ray source configured to emit an x-ray beam along a beam path through at least a portion of an object placement location situated to receive an object for inspection, through an object grating situated along the beam path that includes object grating elements arranged in an object grating pattern, and to a detector situated along the beam path to receive the x-ray beam after propagating through the object grating and the object placement location to produce image data, and with at least one movement stage coupled to the object placement location and/or the x-ray optical arrangement and configured to provide rotational movement between the x-ray optical arrangement and the object placement location to provide a plurality of computed tomography imaging positions for the object by the x-ray optical arrangement: directing the x-ray beam through a first computed tomography imaging position and collecting first image data with the detector; rotating the object placement location and/or x-ray optical arrangement with the movement stage to a second computed tomography imaging position; and directing the x-ray beam through the second computed tomography imaging position and collecting second image data with the detector; wherein the object grating pattern and a positioning of the x-ray source, object grating, and detector in relation to each other are configured to provide a plurality of computed tomography detection modes in each of the first image data and second image data. In some examples, the plurality of detection modes includes absorption, scatter, and/or differential phase. In some examples, the object grating elements include parallel line elements. In some examples, the object grating is situated such that the parallel line elements of the object grating are parallel to a rotational axis of the movement stage. In some examples, the at least one movement stage also provides for translational motion along a line parallel to the rotational axis between the x-ray optical arrangement and the object placement location, wherein the X-ray beam is configured with a height along the rotational axis that is less than a height of the object placement location, and wherein an effective detection height of the detector is less than the height of the object placement location, and wherein a plurality of computed tomography imaging positions comprise position taken at a plurality of different translational positions of the at least one movement stage. In some examples, the effective detection height of the detector includes a sufficient number of pixels to effectively detect at least one entire fringe spacing. In some examples, the optical arrangement comprises a detector grating situated adjacent to the detector and including detector grating elements arranged in a detector grating pattern, wherein the detector grating elements include parallel line elements and the detector grating is situated such that the parallel line elements of the detector grating are perpendicular to the parallel line elements of the object grating. In some examples, the detector grating pattern is configured in relation to the object grating pattern to allow for a beam hardening correction to the image data through a separation of pattern visibility loss due to scatter from pattern visibility loss due to spectral changes. Some examples further include a source grating situated adjacent to the x-ray source and including a source grating pattern configured to produce a plurality of line sources from the x-ray beam. In some examples, the source grating pattern includes a plurality of parallel line elements, and the source grating is situated such that the parallel line elements of the source grating are parallel to the parallel line elements of the object grating. In some examples, the object grating is situated farther from the detector than the object placement location, or, wherein the object grating is situated closer to the detector than the object placement location. In some examples, the object grating is situated less than 0.45L, 0.4L, 0.3L, or 0.2L distance from the x-ray source, where L is the distance between the x-ray source and the detector. Some examples further include, with a processor and memory configured with processor executable instructions, applying a beam hardening correction to the image data through a separation of pattern visibility loss due to scatter from pattern visibility loss due to spectral changes. Some examples further include, with a processor and memory configured with processor executable instructions, performing a computed tomography reconstruction with the image data for one or more of the computed tomography detection modes. In some examples, the performing includes performing the computed tomography reconstruction with the image data for the differential phase computed tomography detection mode, including by Fourier transforming sinograms of the image data, filtering the transformations using a signum filter, and backprojecting the filtered transformations through the object space to obtain a slice; wherein the object grating includes parallel line elements arranged parallel to a rotational axis of the movement stage. In some examples, the performing includes performing the computed tomography reconstruction with the image data for the differential phase computed tomography detection modes, including by integrating differential phase image data, Fourier transforming sinograms of the differential phase image data, filtering the transformations using a ramp filter, and backprojecting the filtered transformations through the object space to obtain a slice. Some examples further include a processor and memory configured with processor executable instructions which provide for: Fourier transforming the detected image data of an object-free region from which pre- and post-exposure grating-only image data can be compared; and fitting Fourier components of the transformed detected image data that are in a Fourier space region around a source grid oscillation peak, to a linear combination of Fourier components of the pre- and post-exposure grating-only image data so that the linear combination can be used as a reference grating image during image processing to extract the phase and scatter images.

According to another aspect of the disclosed technology, a non-transitory computer readable storage medium storing one or more programs, the one or more programs comprising instructions, that are executable by one or more processors operably connected to an x-ray imaging system that comprises: an x-ray optical arrangement that includes an x-ray source configured to emit an x-ray beam along a beam path through at least a portion of an object placement location situated to receive an object for inspection, through an object grating situated along the beam path, and to a detector situated along the beam path to receive the x-ray beam after propagating through the object grating and object placement location and to detect image data, wherein the object grating includes object grating elements arranged in an object grating pattern; and at least one movement stage coupled to the object placement location and/or the x-ray optical arrangement and configured to provide rotational movement between the x-ray optical arrangement and the object placement location to provide a plurality of computed tomography imaging positions for the object by the x-ray optical arrangement; wherein the object grating pattern and a positioning of the x-ray source, object grating, and detector in relation to each other are configured to provide a plurality of computed tomography detection modes in the image data; wherein the instructions cause the x-ray imaging system to collect the image data at one or more of the computed tomography imaging positions.

The purpose of the foregoing summary and the latter abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. Neither the summary nor the abstract is intended to define the invention of the application, which is measured by the claims, nor is it intended to be limiting as to the scope of the claims in any way.

### BRIEF DESCRIPTION OF THE DRAWINGS

FIGS. 1A and 1B include a schematic diagram and a flow chart, respectively, depicting embodiments described herein.

FIG. 2 is a diagram of one embodiment of a computational system for enhanced phase-contrast x-ray imaging and/or beam-hardening correction.

FIG. 3 includes photos of one embodiment of an arrangement of source, object, and detector gratings in an enhanced phase-contrast x-ray imaging system.

FIG. 4 is a screenshot of one example of image acquisition and processing software.

FIG. 5 includes an image of a Fourier transform of an image obtained by enhanced phase-contrast x-ray imaging as described herein.

FIG. 6 is a graph showing corrected scatter ratio as a function of grid frequency (in LPI) for two different particle sizes.

FIG. 7 is a graph showing fringe visibility as a function of endpoint energy and grid spatial frequency.

FIG. 8 is a graph showing visibility reduction as a function of endpoint energy, for a 1.26 cm Al sample (shown with and without the beam hardening correction).

FIG. 9 is a graph showing corrected scatter ratio as a function of particle size, for a 60 kV spectrum. Theoretical predictions are shown as solid lines; measurements are indicated as points with error bars.

FIGS. 10A and 10B include images of a small bag (8″×9″) as in a security screening context. FIG. 10A is a conventional attenuation image. FIG. 10B is an attenuation image with corrected scatter overlay according to embodiments described herein. Propellants present in the bag show scatter, as do business cards, a watch band, and tic tac candies. Chapstick next to the model rocket engine looks similar by shape, but does not exhibit scatter.

FIGS. 11A and 11B are images related to one embodiment of a calibration standard. FIG. 11A is a photograph of ZnO scatter step wedge calibration standards. FIG. 11B are beam-hardening corrected scatter images taken at high energy (160 kV with 2 mm Cu and 2 mm Al filtration) and at low energy (100 kV with 2 mm Al filtration).

FIG. 12 is a line drawing from a photo of an embodiment of a system described herein (absent a source grid). An X-ray tube is at lower right and the beam direction is from lower right to upper left. Filter materials are immediately in front of the x-ray tube; the standard test objects can be seen halfway down the table at upper left; the object and detector grids are also visible in the upper left quadrant. The detector is obscured by the detector grid.

FIG. 13 is a graph showing calculated beam spectra at “high” energy and “low” energy.

FIG. 14 shows line drawings taken from a set of vials containing samples set for end-on imaging and a single vial containing explosive material.

FIG. 15 is a graph showing Dual Energy Results. The y-axis indicates μ_{L}/μ_{H}, which can be related to Z_{eff}, and the x-axis is UH, which can be related to density. Materials A-D represent four different explosive materials and encompass a number of preparations.

FIG. 16 is a graph showing scatter coefficient at low energy v_{L} as a function of μ_{H} (proportional to density). Explosives are labeled by letters A-D.

FIG. 17 is a graph showing scatter coefficient at high energy v_{H} as a function of μ_{H} (proportional to density). Explosives are labeled by letters A-D.

FIGS. 18A and 18B are (18A) close-up of a region of the grating image, showing the vertical grating lines and the longer period Moire pattern. Near the center is a line of bad pixels from a scratch on the detector and (18B) close up of a grating image with both the object and detector gratings, in a section of bad pixels.

FIGS. 19A-19C are regions of a Fourier transform of a grating image.

FIG. 20 is a Fourier transform of a grating image with both an object and detector grid, showing first and second harmonics as well as cross-harmonics.

FIGS. 21A and 21B are images before and after bad pixel correction of a line of bad pixels, respectively.

FIGS. 22A and 22B are images before and after bad pixel correction of a blob, respectively.

FIGS. 23A and 23B are images before and after bad pixel correction of a scatter image of a region illustrated in FIG. 21A.

FIGS. 24A and 24B are images of a region of bad pixels with both a detector and an object grid, and with contributions from the second harmonic peaks of both grids before and after bad pixel correction, respectively.

FIG. 25 is a schematic illustrating grid (i.e., grating) tilt. The beam direction is indicated by the arrow 2502.

FIGS. 26A and 26B are graphs showing modeled flux at the detector from a 0.4 mm spot size 100 kVp source showing values of fringe visibility fits (26A), and plot of fringe visibility (H₁/H₀) along with the second harmonic ratio (H₂/H₀) as a function of rotation angle (26B).

FIGS. 27A and 27B are graphs showing modeled flux at the detector from a 0 mm spot size 100 kVp source showing values of fringe visibility fits (27A), and plot of analytical fringe visibility (H₁/H₀) for perfect (Pb) attenuation and a parallel beam source (27B).

FIG. 28 is a graph showing the modeled fits to fringe visibility measurements with a tilted object grating.

FIG. 29 is a perspective schematic of an example x-ray system that can be used for indirect moiré imaging of objects.

FIG. 30 is a graph showing horizontal and vertical phase positions where indirect moiré images are collected.

FIGS. 31A-33B are phase contrast image pairs comparing uncorrected with corrected images of a test object for absorption (31A-31B), contrast ratio (32A-32B), and phase shift (33A-33B).

FIGS. 34A-34H are schematic depictions of unit cells for object, detector, and analyzer gratings along with various overlaps produced by relative movement between them.

FIGS. 35A-35C are schematic depictions of unit cells object, detector, and analyzer gratings, according to some examples.

FIGS. 36A-36E are indirect moiré images obtained with cross-grid examples.

FIG. 37 is a graph of a spectrum of a x-ray beam after transmission through an aluminum filter.

FIG. 38 is a graph of an example L-shaped set of phase position combinations for an object grating and a detector grating arranged along a beam path.

FIGS. 39A-39C are images of absorption coefficient, visibility ratio, and phase shift produced from simulated data for the broad spectrum source shown in FIG. 37.

FIGS. 40A-40C are images of absorption coefficient, visibility ratio, and phase shift produced from simulated data for a monochromatic (narrow) spectrum source.

FIGS. 41A-41C are images of absorption coefficient, visibility ratio, and phase shift for a set of phase positions.

FIGS. 42A-42C are images of absorption coefficient, visibility ratio, and phase shift after averaging zero phase shift and π-shift analyses.

FIG. 43 is a graph of a set of phase positions including an L-shaped set and a diagonal set.

FIG. 44 is a graph of a set of phase positions including an L-shaped set and a cross-shaped π-shifted set.

FIG. 45 is a graph of a latticed set of phase positions.

FIGS. 46A-46D are absorption coefficient images for different sets of phase positions corresponding to base phase steps, base+diagonal phase steps, base+pi-shift phase steps, and a latticed sequence, respectively.

FIGS. 47A-47D are visibility images for the same sets of phase positions used in FIGS. 46A-46D.

FIGS. 48A-48D are phase shift images for the same sets of phase positions used in FIGS. 46A-46D.

FIG. 49 is a schematic of an example gratings-based phase contrast imaging system in which an x-ray beam is patterned using a grating/grid, and a detector is placed some distance back from the object of interest.

FIG. 50 is a graph of cross section for scatter as a function of relative particle size D′.

FIG. 51A is a schematic of another gratings-based phase contrast imaging system in which a source grid is used to boost visibility of the object grid while using a large focal spot source, and a detector grid, oriented 90° relative to the source and object grids, is used to correct the dark field image for spectral changes. The object and detector grids are imaged directly on the detector.

FIG. 51B is a schematic of another grating-based phase contrast imaging system in which a source grid is used to boost visibility of the object grid while using a collimated beam having a large width in a direction perpendicular to a rotational axis and small height in a direction parallel to the axis of rotation, a wide but short detector, and a translational stage that may be used in forming tomography images at different heights to produce three-dimensional reconstructions with reduced scatter.

FIG. 52-53 are phase images of a scatter step wedge before source grid artifact correction and after source grid artifact correction, respectively.

FIG. 54-55 are a radiograph of the step wedge object before processing, at a single rotation angle, and a picture of the step wedge object, respectively.

FIG. 56A-56B are an extracted zeroth harmonic (absorption) image, which is equivalent to an X-ray radiograph, and an extracted visibility image showing darker regions where there is more small-angle scatter from the zinc-oxide microspheres in the step wedge object, respectively.

FIG. 57A-57B are a radiograph of a plastic columns object before image processing, at a single rotation angle, and a picture of the plastic columns object, respectively.

FIG. 58A-58B are extraction images of the absorption and differential phase information, respectively, from the plastic cylinders object.

FIG. 59A-59B are an absorption sinogram of the scatter step wedge object and a CT reconstruction using filtered back projection (FBP) and a ramp filter, respectively.

FIG. 60A-60B are reconstructions of the scatter signal (100 slice average) and the differential phase signal (10 slice average), respectively, for the step wedge object.

FIG. 61 is a graph of line profiles of the absorption, scatter, and differential phase reconstructions for the scatter step wedge object along column 1000 of the reconstructions.

FIG. 62A-62B are an absorption sinogram of the scatter plastics object and the CT reconstruction, respectively. PVDF is at the top, PTFE is at the left, and PEEK is at the bottom.

FIG. 63A-63B are reconstructions of the scatter signal and the differential phase signal, respectively, for the plastic columns object. PVDF is at the top, PTFE at the left, and PEEK at the bottom.

FIG. 64 is a graph of line profiles of the absorption, scatter, and differential phase reconstructions for the plastic columns object. The line profile is along column 790 of the reconstructions, through the PVDF and PEEK plastics.

FIG. 65A-65B are a radiograph of a 3D-printed object before image processing, at a single rotation angle, and a picture of the object, respectively.

FIG. 66A-66B are an absorption sinogram of the 3D-printed object and the CT reconstruction, respectively.

FIG. 67A-67B are reconstructions of the scatter signal and the differential phase signal, respectively, for the 3D-printed object.

FIG. 68 is a schematic of an example arrangement with approximately 1 m from source (left-most star) to object grid (middle arc) and object, a small imaging region (shown here as a circle, with the axis of rotation perpendicular to the plane of the page), and approximately 1 m from object grid and object to detector (right most arc).

FIG. 69 is another schematic of an example a GBX CT system for security screening. The imaged region is large compared to the source-detector distance, such that the object grid is placed at a different position. The axis of rotation is perpendicular to the plane of the page.

### DETAILED DESCRIPTION

Phase contrast x-ray imaging or gratings-based phase contrast imaging can allow for detection of small deviations in the direction of an x-ray as it passes through a material. These deflections, specifically scatter, can be used to detect texture in a material, such as a powder or a composite, below the imaging resolution of the system. The inventors have determined that measurements at high energies can provide scatter signatures indicative of sub-resolution texture within a sample in order to help identify materials and that the systems, methods, and storage media described herein can be relevant for applications ranging from medical imaging to materials characterization to security screening.

Embodiments described herein can be utilized for discernment of materials properties, especially in non-destructive examination applications. For example, material wetting or compression could be examined (e.g., concrete, plaster, materials that are formed through compression of powders), as could fiber orientations in materials made from carbon fibers or other fibrous materials. Medical applications are also possible, including diagnostic imaging with either radiography or CT. Scatter has been shown to give enhanced contrast for lung structure and for bones. Finally, additional security screening applications may be possible, such as detecting 3-D fabricated parts based on texture or locating powdered materials in mail screening or vehicle screening. Conventional airport security relies on dual-energy x-ray images that can be used to estimate material density and effective atomic number; these two features are relied upon to discriminate threat objects from benign consumer products. However, the estimation from conventional security scanners is often insufficient to effectively distinguish and identify threat objects. Phase contrast imaging can provide additional materials signatures from x-ray measurements: attenuation, which is similar to a conventional x-ray image; refraction or phase, which is based on electron density variations and can be sensitive to low-Z materials; and scatter, which detects the presence of texture (such as powders or composites) below the imaging resolution of the system. The addition of new signatures increases the number of features which can be used for material discrimination, potentially reducing false alarm rates during security screening. Furthermore, one mode may have a lower detection limit than absorption, enabling the detection/identification of additional items and/or features.

Current phase contrast imaging systems typically rely on a grid which produces an x-ray interference pattern (typically with a period of a few microns) and an analyzer grid matched to the undistorted interference pattern. These systems require sub-micron stability and are very difficult to scale to higher, more penetrating energies; they often operate at energies below 100 kVp. When grid fabrication for energies above 100 kVp is possible, it is difficult and expensive. First, the period should be smaller than the coherence length (which decreases as energy increases). Second, the thickness of the attenuating parts of the grid need to be thick enough to stop the x-rays, and this becomes larger at high energies. The net effect is that fabrication with fine feature sizes but extremely large aspect ratios are required; something that is often impractical to manufacture.

For aviation security, phase contrast imaging is not currently used. Dual energy systems provide estimates of material density and effective atomic number to help discriminate benign materials from threats. Adding phase contrast would allow refraction information and texture information to be measured in addition to dual energy, providing a broader basis of material signatures for discriminating materials, and potentially reducing false alarm rates.

Embodiments described herein can detect sub-resolution texture using an object grid as a patterning object in the beam and at a standoff distance from the detector, where the image of the object grid is projected. If a sample containing sub-resolution density variations (such as a powder) is placed near the object grid, the refractive index variations within the object will cause deflections of the x-ray beam, ultimately causing blurring of the projected object grid pattern. This can be described as a reduction in visibility of the pattern.

Importantly, traditional phase contrast imaging occurs at relatively low energies (<100 kVp). Embodiments described herein measure x-ray refraction and scatter at higher energies, while making corrections for spectral effects which can cause spurious scatter-like signals. The embodiments enable the use of high energies which are relevant for NDE applications including airport screening (e.g. 160 kVp). Indeed, the inventors have measured scatter at energies as high as 450 kVp.

The x-ray energies referred to herein are endpoint energies. An x-ray tube produces a polychromatic spectrum of x-rays, with a peak energy defined by the electron energy impinging upon the anode. As the x-rays pass through the object, some energies are more readily absorbed than others, which means that the spectrum behind the object is different than the original spectrum. This in turn changes the visibility of the grid lines. For most materials in the range of energies described herein for x-ray imaging, higher energies are more penetrating than lower energies, which are more readily absorbed in materials. This means that the original visibility of an object grid will tend to be higher for lower energies. When a polychromatic beam passes through an object, the lower energies in the spectrum are more readily absorbed, an effect referred to as “beam hardening”. In this case, the inventors have determined that since the resulting spectrum has more intensity at high energy than the original spectrum, this will cause a reduction in the visibility of the object grid, even in the absence of actual scattering in the object. As the system is run at higher energies and used to interrogate more attenuating objects, the changes in beam spectrum caused by object attenuation also lead to changes in grid pattern visibility, which must be corrected for in order to isolate the visibility reduction due to scatter.

Embodiments described herein differ from other three-grid combination systems and methods at least because some embodiments enable high-energy operation using a polychromatic radiography source (e.g., energies above 100 kVp, 125 kVp, 150 kVp, 160 kVp, 175 kVp, 200 kVp, or 450 kVp, with a spot size, defined as the spatial extent of the region on the x-ray tube anode from which x-rays are emitted, of at least 0.5 mm), in contrast to a synchrotron source or a conventional source operated at lower energies and/or spot sizes. In certain embodiments, the source operates with a current ranging from 0.1 to 1000 milliamps.

In particular, embodiments described herein differ from a three-grid Talbot-Lau interferometer, which uses a source grid to increase spatial coherence, an object grid that forms an interference pattern, and an analyzer grid that detects small changes in the very small interference pattern. In other words, the object grid is a phase element and sets up an interference pattern that impinges on the analyzer grid in order to help detect deviations in the interference pattern without resolving it directly. The source grid in the Talbot-Lau configuration is required to form a sufficiently smooth wave front to establish an interference pattern and the required coherence. In contrast, embodiments described herein utilize large spot sizes (at least 0.5 mm) while retaining the ability to not blur the pattern image and to improve resolution, not coherence. The Talbot-Lau analyzer grating is aligned with the object grating and matches the projected object grating period. Another distinction of present embodiments compared to a Talbot-Lau-style interferometer is the absence of a requirement for gratings which are both fine (period of 5 microns or less) and extremely high aspect ratio (often 10:1, and up to 100:1 for 100 keV), sub-micron alignment and stability, and highly precise (sub-micron) stepping of an analyzer grid placed near the detector. This combination is difficult and impractical for many applications for conventional systems. In contrast, some embodiments described herein utilize gratings having grating elements comprising parallel channels with an aspect ratio less than 10:1, 8:1, 5:1, or 3:1 when the source operates at an energy of at least 100 kVp. In certain embodiments, the gratings can have a scale greater than a 2 micron period, a 5 micron period, a 10 micron period, a 25 micron period, a 50 micron period, or a 100 micron period, which can enable different fabrication methods that are much easier.

In summary, the inventors have determined that the combination of a directly imaged, attenuation-based object grid, the use of a source grid to improve imaging of the object grid using a high-energy polychromatic source with a large spot size, and the use of a stationary detector grid having gratings oriented substantially orthogonally to that of the object grid, addresses the artifacts and beam hardening effects that limit the quality and discriminatory power of high-energy x-ray imaging that includes phase contrast. The object grid is visible on the detected image and is, therefore, sufficiently coarse to be directly visualized on the detector. However, this coarseness can reduce scatter sensitivity. In certain embodiments, the object grid is positioned substantially equidistant between the source and detector in order to optimize contrast for most samples by providing 2× magnification of the grid on the detector. Furthermore, most high energy sources have a large x-ray tube spot size, so their use is enabled by the added source grid. Finally, high energy applications typically involve highly attenuating objects, making the beam hardening correction critical for accurate results, which requires the detector grid. Thus, all three grids operate synergistically to enable embodiments disclosed herein.

The explanations of terms and abbreviations herein are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, distances, energies, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximations unless the word “about” is recited.

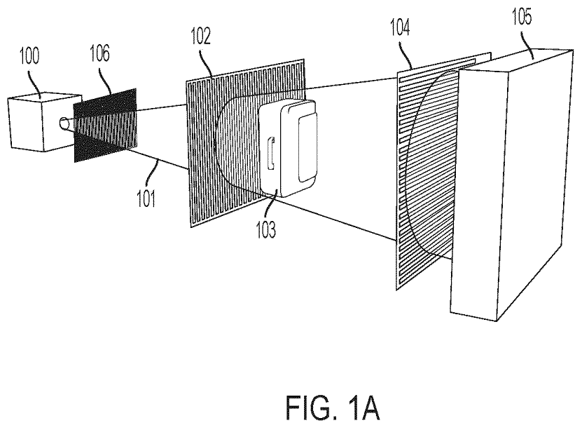

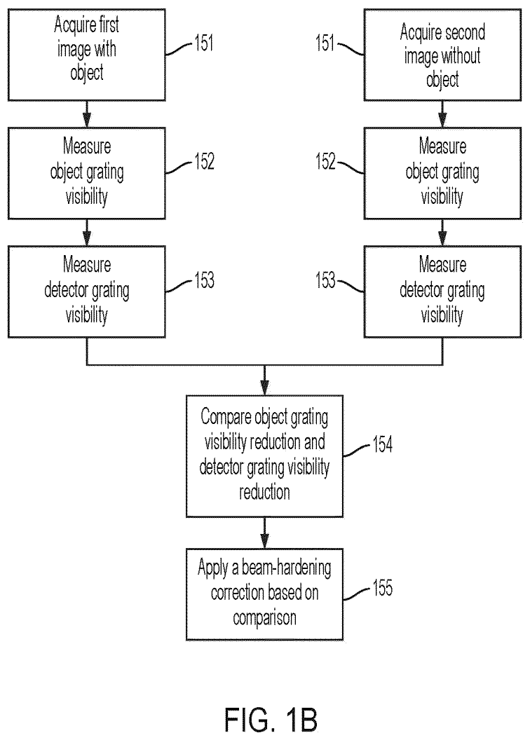

Referring to FIG. 1A, a diagram summarizes one embodiment of a system for beam-hardening-corrected, phase-contrast, x-ray imaging. As illustrated, the embodiment comprises three x-ray anti-scatter grids having parallel line grating elements. The source grid 106 is placed at the x-ray source 100, the object grid 102 is in proximity to an object 103 being imaged, and the detector grid 104 abuts the detector 105. The source and detector are placed some distance apart, with an object being imaged located between the source and the detector. The source is operated at high energies and generates a large spot size. The object grid can be on the source or detector side of the object and the object grid lines are spaced at period, P_{object}. The source grid is placed near the x-ray source with period, P_{source} substantially equal to P_{object}·[(L₁+L₂)/L₁], wherein L₁ and L₂ are distances from the source to the object and from the object to the grid, respectively. In certain embodiments, the distance between the source and the object and the distance between the object and detector are substantially equivalent. In certain embodiments, the detector grating has a periodicity that is equivalent to that of the source grating. The fundamental relationship is between P_{source} and P_{object}, to get the patterns to overlay when projected onto the detector. As a matter of convenience, efficiency, and/or optimization to set P_{detector} equal to the projected P_{object} to make the regions around their respective Fourier peaks are about the same size in Fourier space. When L₁ and L₂ are equal, P_{source} can equal P_{detector}.

The source grid can compensate for a large x-ray tube spot size, which can be detrimental to being able to resolve the lines of the object grid. In other words, the source grid can allow the object grid to be imaged on the detector more clearly. The detector grid is placed at the detector and the orientation of the grating elements is substantially 90 degrees rotated relative to those of the object grid. With regard to rotation of the detector grating elements, substantially can refer to an error of ±1, ±2, or ±5 degrees. In certain embodiments, the error in detector grating rotation angle should be less than that which would avoid overlap of the first harmonics in Fourier space.

The object grid and detector grid have substantially the same attenuating factor. With regard to the attenuating factor, substantially refers to an error of ±1%, ±3%, ±5%, or ±10%. For example, the object and detector grids can comprise the same material and same thickness. The detector grid is used to correct for artifacts caused by beam hardening, where the spectrum of the beam is changed by attenuating objects. When the object grid is substantially equidistant from the source and detector, the source and detector gratings can have substantially the same grating element period.

FIG. 1B includes a flowchart summarizing one example of a computer-implemented method of beam-hardening correction of phase-contrast x-ray imaging. The method can be embodied by non-transitory, computer-readable storage media storing instructions that can be executed to perform the method. In the illustrated example, first and second images are acquired with and without the object to be imaged 151. The “first image” and “second image” terms do not refer to chronological sequence; the images can be acquired in any order. The object grating visibilities are measured in both images 152. Similarly, the detector grating visibilities are measured in both images 153. The visibilities are compared 154 to determine the object grating visibility reduction and the detector grating visibility reduction. Based on the comparison of the reductions 154, a beam-hardening correction is applied 155 to yield a corrected phase-contrast x-ray image.

Non-transitory as used herein when referring to a computer-readable storage medium, is a limitation of the medium itself (i.e., tangible, not a propagating electromagnetic signal) as opposed to a limitation on data storage persistency. The term is not intended to otherwise limit the type of physical computer-readable storage device that is encompassed by the phrase computer-accessible medium or memory. For instance, the terms “non-transitory computer readable medium” or “tangible memory” are intended to encompass types of storage devices that do not necessarily store information permanently, including but not limited to, computer-readable media that store data only for short periods of time and/or only in the presence of power, such as register memory, processor cache and Random Access Memory (RAM). Program instructions and data stored on a tangible computer-accessible storage medium in non-transitory form may further be transmitted by transmission media or signals such as electrical, electromagnetic, or digital signals, which may be conveyed via a communication medium such as a network and/or a wireless link.

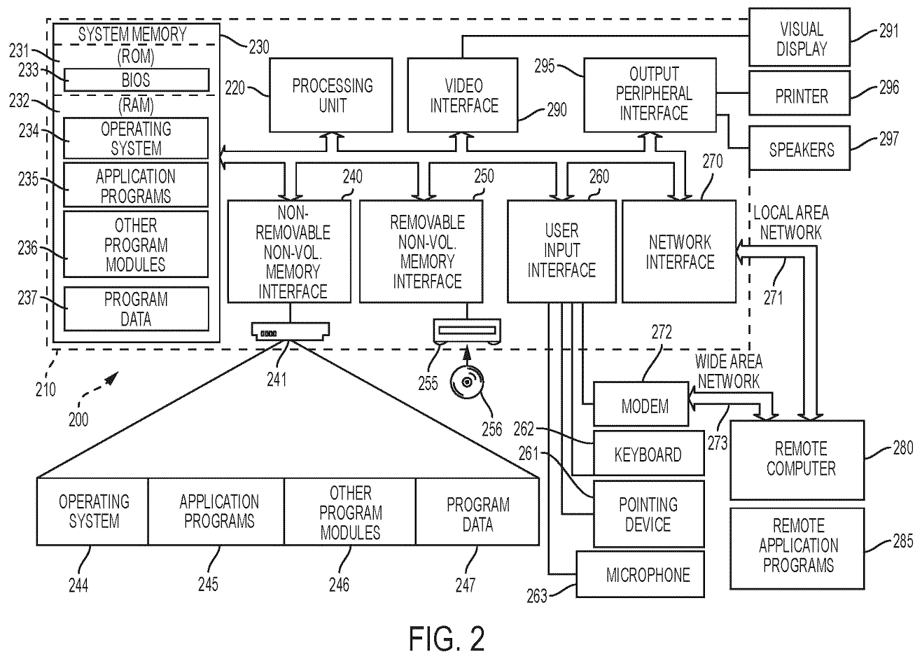

FIG. 2 is one embodiment of a computational system or computing environment to which an enhanced phase-contrast x-ray imaging system can be operably connected. Alternatively, the computational system can be integrated within an enhanced phase-contrast x-ray imaging system. In one example, a computing environment such as shown in FIG. 2 can be used to control operation of the imaging system. The computing environment can further be used to acquire images and to perform beam-hardening corrections as described elsewhere herein.

With reference to FIG. 2, an example system for implementing some embodiments includes a general-purpose computing device in the form of a computer 210. Components of computer 210 may include, but are not limited to, a processing unit 220 (which is not limited to CPUs, but can comprise GPUs), a system memory 230, and a system bus 221 that couples various system components including the system memory to the processing unit 220. The system bus 221 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus. Memory and programs described herein be deployed in corresponding portions of FIG. 2.

Computer 210 typically includes a variety of computer readable media. Computer readable media can be any available media that can be accessed by computer 210 and includes both volatile and nonvolatile media, removable and non-removable media. By way of example, and not limitation, computer readable media may comprise computer storage media and communication media. Computer storage media is different from, and does not include, a modulated data signal or carrier wave. It includes hardware storage media including both volatile and nonvolatile, removable and non-removable media implemented in any method or technology for storage of information such as computer readable instructions, data structures, program modules or other data. Computer storage media includes, but is not limited to, RAM, ROM, EEPROM, sash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 210. Communication media typically embodies computer readable instructions, data structures, program modules or other data in a transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer readable media.

The system memory 230 includes computer storage media in the form of volatile and/or nonvolatile memory such as read only memory (ROM) 231 and random-access memory (RAM) 232. A basic input/output system 233 (BIOS), containing the basic routines that help to transfer information between elements within computer 210, such as during startup, is typically stored in ROM 231. RAM 232 typically contains data and/or program modules that are immediately accessible to and/or presently being operated on by processing unit 220. By way of example, and not limitation, FIG. 2 illustrates operating system 234, application programs 235, other program modules 236, and program data 237.

The computer 210 may also include other removable/nonremovable volatile/nonvolatile computer storage media. By way of example only, FIG. 2 illustrates a hard disk drive 241 that reads from or writes to non-removable, nonvolatile magnetic media, and an optical disk drive 255 that reads from or writes to a removable, nonvolatile optical disk 256 such as a DVD or other optical media. Other removable/non-removable, volatile/nonvolatile computer storage media that can be used in the exemplary operating environment include, but are not limited to, magnetic tape cassettes, sash memory cards, DVDs, digital video tape, solid state RAM, solid state ROM, and the like. The hard disk drive 241 is typically connected to the system bus 221 through a nonremovable memory interface such as interface 240, and optical disk drive 255 are typically connected to the system bus 221 by a removable memory interface, such as interface 250.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

The drives and their associated computer storage media discussed above and illustrated in FIG. 2, provide storage of computer readable instructions, data structures, program modules and other data for the computer 210. In FIG. 2, for example, hard disk drive 241 is illustrated as storing operating system 244, application programs 245, other program modules 246, and program data 247. Note that these components can either be the same as or different from operating system 234, application programs 235, other program modules 236, and program data 237. Operating system 244, application programs 245, other program modules 246, and program data 247 are given different numbers here to illustrate that, at a minimum, they are different copies.

A user may enter commands and information into the computer 210 through input devices such as a keyboard 262, a microphone 263, and a pointing device 261, such as a mouse, trackball or touch pad. Other input devices (not shown) may include a joystick, game pad, satellite dish, scanner, or the like. These and other input devices are often connected to the processing unit 220 through a user input interface 260 that is coupled to the system bus, but may be connected by other interface and bus structures, such as a parallel port, game port or a universal serial bus (USB). A visual display 291 or other type of display device is also connected to the system bus 221 via an interface, such as a video interface 290. Video interface 290 can comprise a graphics card having a GPU. The GPU be used for computations. In addition to the monitor, computers may also include other peripheral output devices such as speakers 297 and printer 296, which may be connected through an output peripheral interface 295.

The computer 210 is operated in a networked environment using logical connections to one or more remote computers, such as a remote computer 280. The remote computer 280 may be a personal computer, a hand-held device, a server, a router, a network PC, a peer device or other common network node, and typically includes many or all of the elements described above relative to the computer 210. The logical connections depicted in FIG. 2 include a local area network (LAN) 271 and a wide area network (WAN) 273, but may also include other networks. Such networking environments are commonplace in offices, enterprise-wide computer networks, intranets and the Internet.

When used in a LAN networking environment, the computer 210 is connected to the LAN 271 through a network interface or adapter 270. When used in a WAN networking environment, the computer 210 typically includes a modem 272 or other means for establishing communications over the WAN 273, such as the Internet. The modem 272, which may be internal or external, may be connected to the system bus 221 via the user input interface 260, or other appropriate mechanism. In a networked environment, program modules depicted relative to the computer 210, or portions thereof, may be stored in the remote memory storage device. By way of example, and not limitation, FIG. 2 illustrates remote application programs 285 as residing on remote computer 280. It will be appreciated that the network connections shown are exemplary and other means of establishing a communications link between the computers may be used.

### Examples and Comparisons

To further illustrate certain embodiments of the disclosed methods, systems, and computer-readable storage media, and to provide various comparative analyses and data, below are some examples with comparison test data.

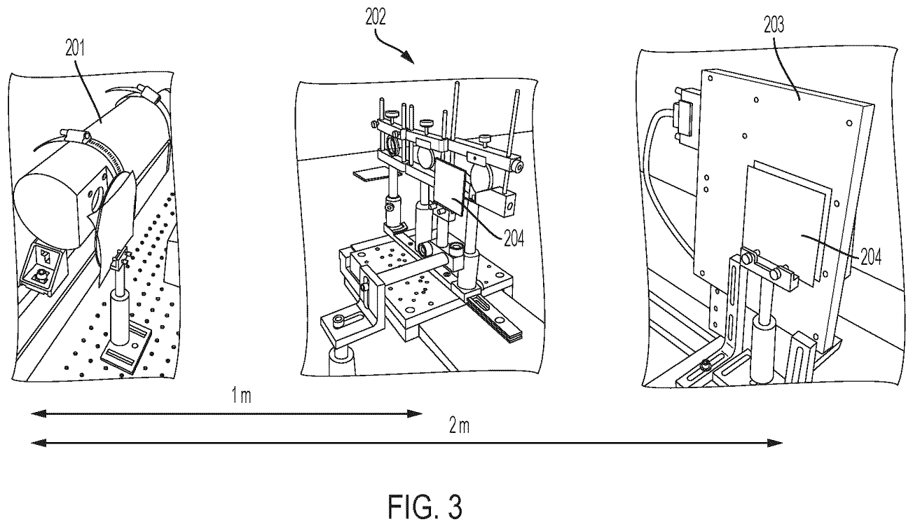

Photos of a basic system, including a source, object grid, and detector are shown in FIG. 3. The x-ray source 201 used was a Comet MXR-160HP/11 x-ray tube with a maximum energy of 160 kV. The maximum power was 1800 W with a 1 mm spot size or 800 W with a 0.4 mm spot size. In some embodiments, when the spot size is too large to resolve the object grid on the detector, a source grid is necessary. The relationship describing the spot size above which the ability to resolve the object grating is reduced can be expressed as w_{source}≥ (L₁+L₂)*P_{obj}/L₂, where w is the spot size of the source, L₁ is the source-to-object distance, and L₂ is the object-to-detector distance. Test data shown below used 1 mm Al and 0.1 mm Cu to filter the spectrum of the beam and reduce the flux at very low photon energies. The working distance from source to detector 203 was set at 2 m, and the detector used initially was a CMOS X-ray detector (e.g., Shad-o-box® 4k) with a 10 cm×10 cm field of view, 48 μm pixel pitch, and a Gd₂O₂S:Tb scintillator (Teledyne DALSA®). The object and object grid 202 were placed halfway between the source and detector, resulting in a two-times magnification and good sensitivity to the small angular deflections by the sample. The beam pattern was created with commercial anti-scatter grids 204 used for medical imaging, consisting of parallel line patterns between 85 and 285 lines per inch (LPI). Typically, the grids are made of lead and aluminum sheets interspersed, but the finest grid was made with carbon fiber between lead sheets. A second grid, approximately half the object grid's spatial frequency, was oriented such that the grid lines were perpendicular to those of the object grid and was placed directly in front of the detector in order to correct for beam hardening, as described elsewhere herein.

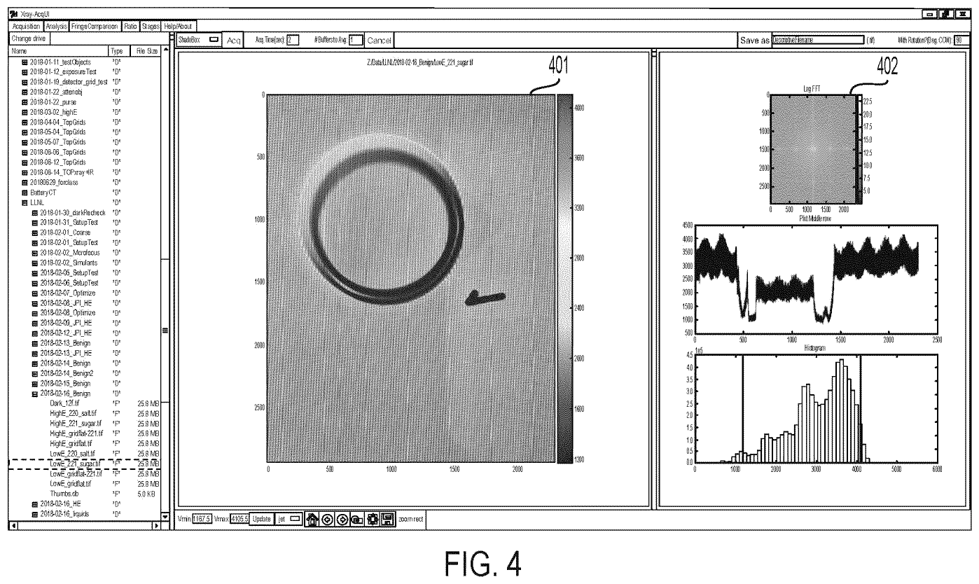

Custom software was developed to handle both data acquisition and extraction of absorption, phase, and scatter images. Frames were acquired from the detector, summed, and saved as floating-point tiff files; this file type allowed viewing as an image while preserving raw numbers from the detector and the full bit-depth of the detector. The interface to the data acquisition and processing software is shown in FIG. 4.

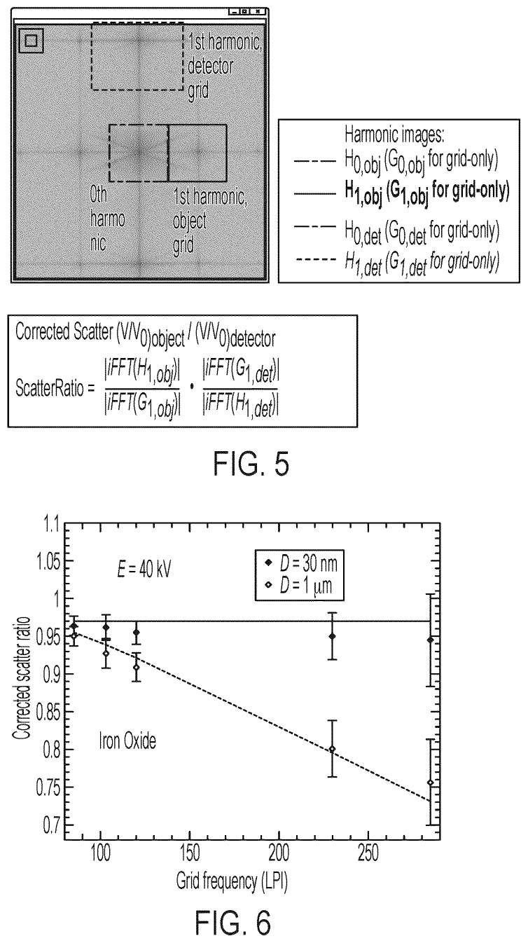

Raw images 401 resembled a normal attenuation image, but with fine vertical and horizontal lines visible from the object and detector grids. A Fourier-based processing method was used. First, a Fourier transform 402 is taken of the image. For a system with parallel line grids, peaks will be visible in the Fourier transform corresponding to the spatial frequency of the grid. In FIG. 5, peaks to the right and left of center correspond to the first harmonic associated with the object grid (which was oriented with its grid lines vertical), and the peaks at top and bottom are the first harmonic of the detector grid (which was oriented with its lines horizontal. The region around the first harmonic when an object is present is compared with the same region a grid-only image (no sample). Pattern displacements of the object grid were determined based on the phase values in a region of the Fourier transform around the first harmonic. These were interpreted as refraction and formed the differential phase contrast image. Pattern visibility, V, is calculated based on the ratio of the inverse Fourier transform of the region around the first harmonic to the inverse Fourier transform of the region around the zeroth harmonic, but can be more simply understood as the amplitude of the projected grid pattern divided by the mean. Pattern visibility reduction, V/V₀, which compares pattern visibility with an object present to pattern visibility without, was interpreted as scatter. Note, visibility can be reduced not only by scattering effects but also by beam hardening, since a harder spectrum may result in less modulation of the object grid. To correct for this, a detector grid was chosen that has similar attenuation properties as the object grid so that it would be similarly affected by beam hardening. This grid was placed as close as possible to the detector to minimize the effects of scatter on the projected pattern. The ratio of the scatter image to the apparent scatter in the detector grid image produced a correction to remove the portion of the visibility loss due to beam hardening.

The first study performed was a test of the system sensitivity to the object grid spatial frequency. Calibration standards were constructed of iron oxide (i.e., Fe₃O₄) nanoparticles dispersed in epoxy at a 20% volume fraction. Objects were 6 mm thick and were constructed with two different sizes of particles: 30 nm and 1 μm. A source spectrum with a peak energy of 40 kV was used. Results are shown in FIG. 6, which shows the corrected scatter ratio, which is the visibility reduction at the object grid after correcting for beam hardening as a function of grid frequency (in lines per inch). For the 1 mm particle sample, as the grid frequency was increased, the scatter increased approximately linearly. For the 30 nm particles, the response was independent of grid frequency. This is consistent with theoretical descriptions of the measurement process where the x-ray interactions are due to Small Angle X-ray Scattering (SAXS) and the response of the system is characterized by a parameter known as the correlation length. The correlation length of the measurement at 40 kV ranges between 50 and 300 μm depending on the grid frequency. For particles much larger than the correlation length, scatter signal increases with larger grating spatial frequencies, while for samples much smaller than the correlation length, scatter signal should be independent of gratings frequency.

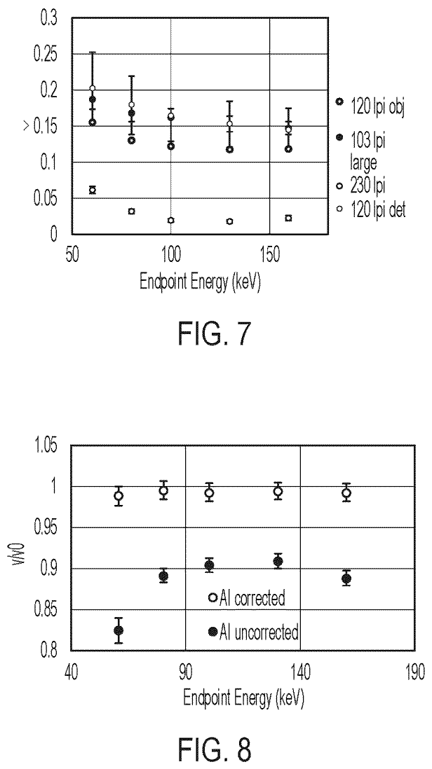

For explosives detection, particles larger than the correlation length are of primary interest. In certain embodiments, texture is considered to be in the 1-1000 microns for what is defined as a powder. The correlation length is inversely proportional to energy, so increasing the spatial frequency of the object grid as much as possible will improve measurement sensitivity. For signatures of explosive and benign materials that, when textured, typically have variations on length scales ranging from microns to millimeters in size, a finer object grid (higher spatial frequency) is generally advantageous. However, as grid frequency decreases, imaging the projected grid pattern becomes more difficult. This can be caused by the finite size of the x-ray source region in the tube, finite resolution at the detector, and by limited attenuation in a finely patterned grid (as spatial frequencies increase. Therefore, higher aspect ratio fabrication is required in order to retain sufficient thickness to modulate the beam). To examine these effects, we measured the visibility with no object present for several grids and measurement geometries. Higher visibilities indicate a larger fraction of the beam intensity is available for detecting refraction and scatter; lower visibilities will lead to noisier measurements. FIG. 7 is a graph that shows visibility as a function of endpoint energy for 103, 120, and 230 LPI grids placed halfway between source and detector, and a 120 LPI grid placed near the detector. Overall visibility values range from less than 0.05 up to 0.30. Visibility decreases with energy, as the beam becomes more penetrating, but changes relatively slowly at higher energies. The coarsest grid, at 103 LPI, showed the highest visibilities and the finest grid, 230 LPI, showed the lowest. For the 120 LPI grid, measurements were done with the grid halfway between source and detector, where pattern visibility was equally sensitive to resolution limitations due to the source and to the detector, and with the grid near the detector, where resolution was strongly dependent on the detector and independent of the source spot size. The grid showed higher visibility at the detector position, indicating that for grids halfway between source and detector, the source spot size was the dominant factor in reducing visibility.

The beam hardening correction was tested as a function of energy using a 1.26 cm thick section of aluminum (for reference, the mean free path for attenuation in Al ranges from 0.68 cm at 40 keV to 2.7 cm at 160 keV). This was selected as a material that was expected to be homogeneous over any texture length scales that the measurement would be sensitive to (e.g., nm to μm). Fringe visibility reduction (V/V₀, where V₀ is the grid visibility without an object present) is plotted in FIG. 8 as a function of endpoint energy, both for the object grid alone (Al uncorrected), and for the object grid after beam-hardening correction by the detector grid (Al corrected). We can verify that, although the raw object grid (uncorrected) shows substantial decreases in fringe visibility, particularly at low energies, the correction by the detector grid results in calculated visibility reduction numbers independent of peak energy and consistent with a ratio of 1, indicating a homogeneous, non-scattering material, as expected.

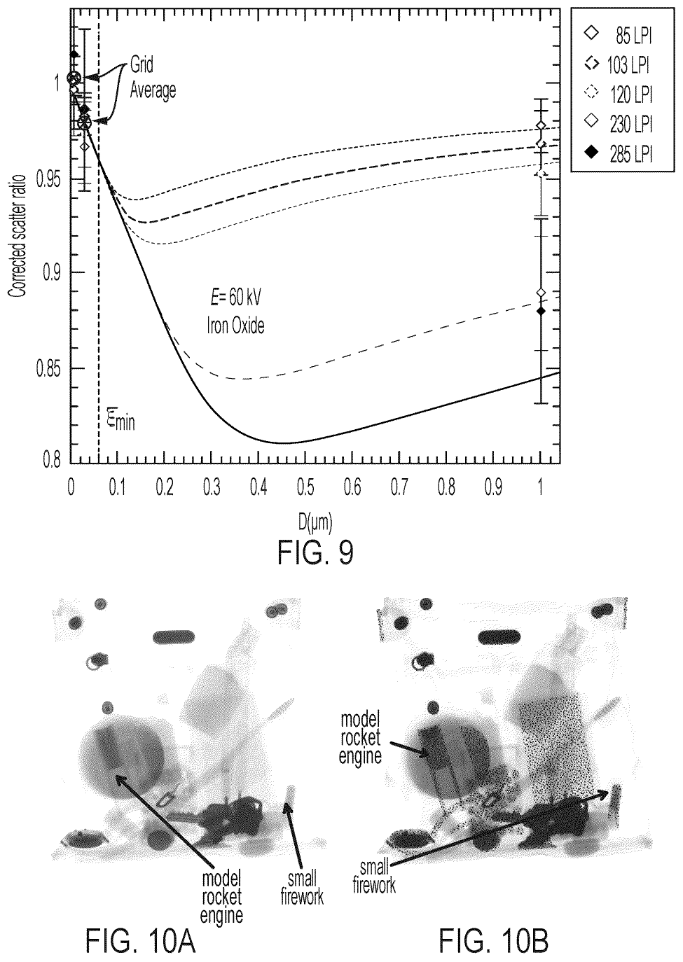

The scatter for a typical gratings-based setup is expected to be dominated by small angle x-ray scattering (SAXS), a mechanism for elastic scattering that produces a spectrum as a function of a scattering vector reflecting the distribution of spatial features in the sample. For the gratings-based measurement, results are not resolved as a function of scattering vector, but sensitivity is greatest near the scattering angle defined by the object grid-to-detector distance, d, and the size of the projected grid period, P_{projected}. The momentum transfer associated with this scattering angle can be related to a correlation length in the material, ξ_{corr}=d*hc(P_{projected}·E), where E is the photon energy and E/hc is the photon wavelength; this correlation length is closely related to the particle size that produces peak scatter intensity. In FIG. 9, we plot corrected scatter ratio as a function of particle size for a 60 kV spectrum. Solid lines are theoretical estimates for a range of grating frequencies. As grating frequency was increased, the effects on scatter ratio become larger, and the correlation length increased as well, leading to peak scattering at increasingly large particle size. Measured results are shown for iron oxide nanoparticles at 7 nm, 30 nm, and 1000 nm mean particle size; they were generally consistent with theoretical predictions.

The measurements shown established that fringe visibility is possible at energies up to 160 kV and illustrated the importance of correcting for beam hardening in interpreting fringe visibility reduction as scatter. A tradeoff is demonstrated between increased scatter signal for higher spatial frequency grids and reduced overall visibility as grid frequency is increased, which will lead to lower signal-to-noise.

A source grid can improve the ability to resolve the object grating. The source grid is aligned parallel to the object grid, with half the spatial frequency (when the object grid was placed substantially equidistant between the source and detector); this results in multiple projected images of the object grid that overlay at the detector. For a 160 kVp spectrum and a 1 mm source spot size, adding a source grid with 50% duty cycle and a period of 4.7 lines per mm increased the visibility of a 9 lines per mm object grid placed 1 m downstream of the source and 1 m upstream of the detector by approximately 4×. This approach allows the use of a larger spot size and therefore higher flux.

Referring to FIG. 10, a composite image of a small bag (8″×9″), taken at 160 kV using a large spot size and a source grid to improve object grid visibility is shown according to embodiments described herein. The image was acquired using a system comprising a polychromatic source (e.g., Comet x-ray tube) run with a 1 mm spot size, a 103 LPI source grid, 210 LPI object grid, and 103 LPI detector grid oriented perpendicular to the other two for beam hardening corrections, used with the CMOS detector. The image on the left is an attenuation image. The image on the right is an attenuation image with scatter overlay (color). Propellants present in the bag show scatter, as do business cards, a watch band, and tic tac candies. Chapstick next to the model rocket engine looks similar by shape, but does not exhibit scatter.

In order to characterize the performance and verify consistency of an imaging system, particularly the ability to detect texture, a set of calibration standards was developed. The first type of calibration standard comprises a scatter test object and provides a stable and repeatable means for measuring scatter signal across different systems. The inventors determined that the scatter test object, which was stable and robust with well characterized small-scale structure, was beneficial so that the efficacy of different x-ray systems could be tested. The scatter test object can have sufficient contrast for use at high energies, when the cross section for elastic scatter is relatively small. One embodiment of the scatter test object calibration standard comprises a block of polymer with microparticles or nanoparticles dispersed evenly within it. The particles can comprise metal and/or metal oxide. The particles have a known size distribution. The polymer block can be fabricated with a series of steps or geometric features of different thickness. The resulting object provides a measure of x-ray scattering as a function of thickness, over a wide range of imaging systems and x-ray energies, and which is stable and robust. The testing and calibration can be particularly advantageous for certain applications including explosives detection and medical imaging. In such applications, the scatter test object can comprise a scatter-imaging phantom. The phantom is an object having the same scatter qualities and/or properties as a material in which one is interested in imaging.

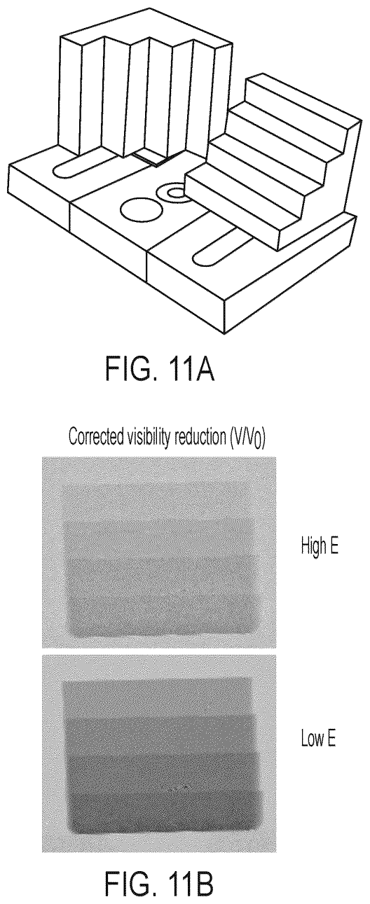

In some embodiments, metal or metal oxide microparticles or nanoparticles were fixed in a polymer. Particles with a well-defined size distribution are commercially available. This is important because the scatter signal exhibits sensitivity to the size of the particles or texture. A polymer (e.g., epoxy) is robust and stable over time and adds no additional scatter signal. The metal or metal oxides have a sufficiently high density that the x-ray refractive index change between the particles and the polymer produce a strong scattering signal. After testing numerous metal and metal oxide nanoparticles and microparticles, ZnO was found to disperse evenly in epoxy, and scatter step-wedges were created out of 20 vol % ZnO particles fixed in epoxy. Blocks were created with 1 μm particles, and with a distribution of particles 5 μm and below; steps were cut to be approximately 6 mm thick with a maximum thickness of 25 mm. The scatter step wedges are shown in FIG. 11A, along with scatter images (see FIG. 11B) at two different energies. When transitioning between different gratings-based systems, or different energies, contrast-to-noise was measured on the step wedges and compared. The scatter test object calibration standard is not limited to a step wedge shape. A wedge with graded thickness is an example of an alternative shape. Further still, the scatter test object can comprise one or more shapes having a constant thickness. A plurality of constant thickness calibration standards, each with different thickness, different particle loadings, and/or particle sizes can be used as an alternative.

Another type of calibration standard comprises a beam hardening test object for phase contrast x-ray imaging, which can be used to test for beam hardening artifacts that can adversely affect the scatter measurement and ensure the artifacts have been properly removed. The use of the beam hardening test object calibration standard relies on the fact that it does not have density fluctuations at length scales to which the measurement is sensitive—that the materials in the calibration standard are homogeneous. This provides a baseline expectation that data taken with the test device will, if properly corrected for beam hardening, indicate no additional fringe visibility loss due to texture. For many applications, a beam hardening correction will be applied for multiple materials, spanning much of the periodic table, and for materials with a wide range of attenuation values. The calibration standard is designed to contain multiple homogeneous materials across a range of atomic number, with the thickness of each material selected so that a moderate amount of attenuation (10% to 90% of the original beam) is present.

One embodiment of the beam hardening test object calibration standard can comprise three or more materials that are each homogeneous, with no large density variations on length scales between 10 nm and 200 microns, and represent a range of atomic numbers. The materials are machined to a thickness suited to the energy of the x-rays used, such that 10-90% of the beam intensity is transmitted through the object. A corrected phase contrast measurement, as described elsewhere herein, is performed with corrections for spurious signals due to spectral changes during attenuation, and the resulting scatter image of the calibration standard will be consistent with background if the correction is successful.

In some embodiments, beam hardening test object comprises approximately one mean free path at 160 kV of aluminum (28 mm), stainless steel (7 mm), copper (5.5 mm), and tin (1.0 mm). This gave a range of Z, and substantial attenuation, over which to test the beam hardening correction. The beam hardening correction can correct partially the visibility reduction observed in the calibration standard materials, in contrast with the complete correction observed with the 12.5 mm Al sample. This appears to be related to the relatively high attenuation of the calibration standard. Known homogeneous material samples (such as water) show a corrected scatter value consistent with homogeneity. Accordingly, there is no issue with the calibration standard significantly impacting the measurements of the explosive and benign materials.

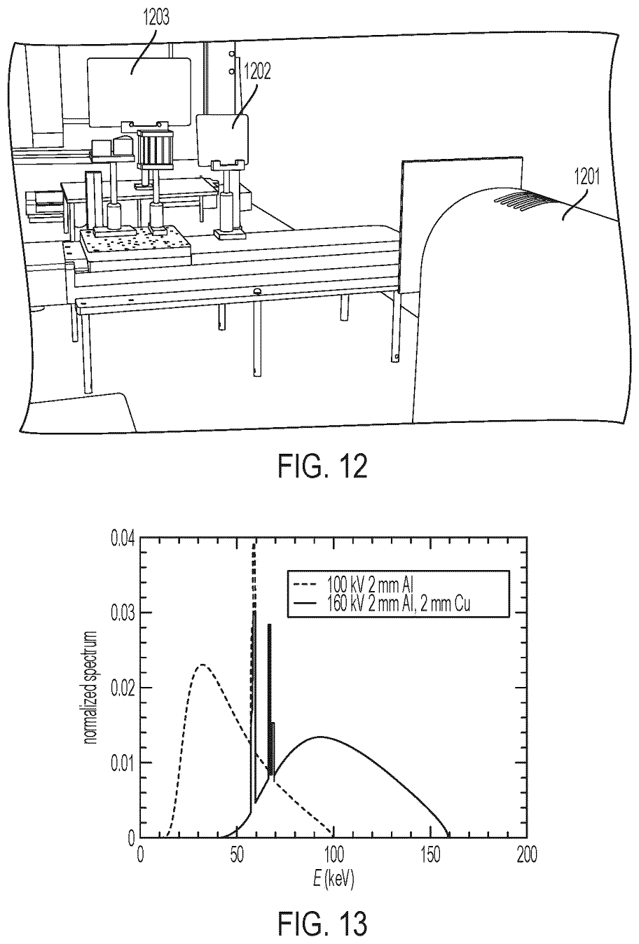

Measurements of a variety of materials, including threat and non-threat materials, were conducted in collaboration with Chuck Divin, Sabrina De Piero, Larry McMichael, and Harry Martz at Lawrence Livermore National Laboratory. These measurements were performed using a microfocus x-ray tube (Hamamatsu L12161-07). The nominal spot size at max current was 50 μm. The final measurement configuration is shown in FIG. 12 and consisted of the Hamamatsu microfocus tube 1201 operated at 0.5 mA current and 50 μm spot size, an object grid 1202 with 285 LPI (JPI Healthcare), and a detector grid 1203 with 120 LPI (Kiran Medical). Measurement time was necessarily very long-10 minutes, or 300 mA·s. This was selected and verified to be well above the range where noise in the images is dominated by counting statistics, in order to emphasize the physical signatures. The small spot size provided by the microfocus tube necessitated longer measurement times. In some embodiments, high-energy, large spot size measurements described herein have measurement times that are less than 10 minutes, 8 minutes, 6 minutes, 5 minutes, 3 minutes, 2 minutes, 1 minute, or 30 seconds.

Data was acquired for two different spectra, chosen to be similar to spectra used for dual-energy measurements in current checkpoint screening. Calculated spectra are shown in FIG. 13. The high energy spectrum, in blue, had an endpoint energy of 160 kV (but was reduced to 150 kV with the Hamamatsu source), 2 mm of copper and 2 mm of aluminum filtration. The average correlation length for this system, with the 285 LPI grid, was 90 nm. The low energy spectrum, shown in red, had an endpoint energy of 100 kV, 2 mm aluminum filtration, and an average correlation length of 170 nm.

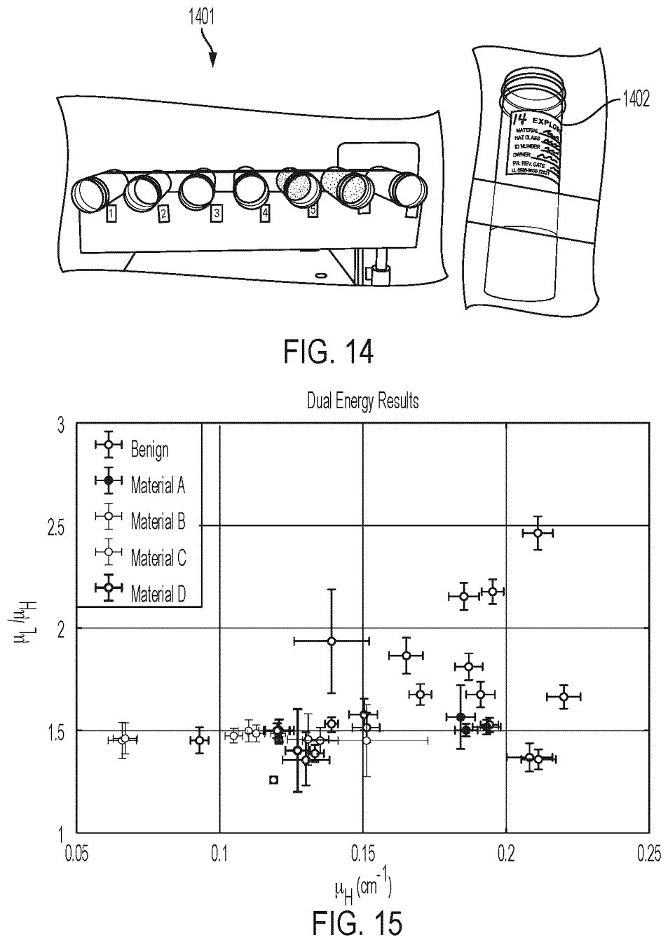

The test dataset consisted of over 20 different benign materials, selected with knowledge of items typically found in baggage, and with a wide range of densities, effective atomic numbers, and including several items which were powdery or had other density variations. Four threat materials were selected, all with some level of mesoscale texture. Three of the materials were powders, with a range of grain sizes and preparation methods, and one was a moldable. Materials were placed in plastic cylinders 3 cm in diameter and 2-3 cm thick, had a total mass of 15-30 g per sample, and were imaged end-on to produce a large area with uniform thickness. An example vial 1402 and set of samples 1401 assembled for imaging are shown in FIG. 14.

TABLE 1

| | μ_{H} | μ_{L} | | ν_{H} | ν_{L} |
|---|---|---|---|---|---|
| Material | (cm⁻¹) | (cm⁻¹) | μ_{L}/μ_{H} | (cm⁻¹) | (cm⁻¹) |
| raspberry | 0.208 ± | 0.285 ± | 1.37 | −2.00E−03 ± | −2.25E−03 ± |
| jelly | 0.008 | 0.010 | | 1.38E−02 | 2.49E−03 |
| strawberry | 0.211 ± | 0.287 ± | 1.36 | −4.44E−05 ± | −9.30E−04 ± |
| jelly | 0.006 | 0.007 | | 1.33E−02 | 2.45E−03 |
| coconut | 0.170 ± | 0.285 ± | 4.67 | 9.20E−05 ± | −1.32E−03 ± |
| oil | 0.004 | 0.005 | | 1.19E−02 | 2.64E−03 |
| rubber | 0.119 ± | 0.150 ± | 1.27 | −1.86E−04 ± | 2.83E−04 ± |
| cement | 0.001 | 0.001 | | 1.13E−02 | 2.02E−03 |
| Vaseline | 0.130 ± | 0.177 ± | 1.36 | 5.21E−04 ± | 2.97E−04 ± |
| | 0.008 | 0.013 | | 5.81E−03 | 2.53E−03 |
| Colgate | 0.191 ± | 0.320 ± | 1.67 | 3.63E−03 ± | 1.46E−02 ± |
| | 0.005 | 0.008 | | 6.45E−03 | 3.08E−03 |
| Olay | 0.165 ± | 0.308 ± | 1.87 | 9.21E−03 ± | 3.33E−02 ± |
| sunscreen | 0.006 | 0.010 | | 6.44E−03 | 3.16E−03 |
| pure | 0.194 ± | 0.297 ± | 1.54 | −5.31E−04 ± | −9.83E−04 ± |
| honey | 0.003 | 0.004 | | 6.88E−03 | 2.60E−03 |
| apricot | 0.150 ± | 0.237 ± | 1.58 | 2.69E−04 ± | 4.84E−03 ± |
| scrub | 0.005 | 0.008 | | 7.31E−03 | 3.37E−03 |
| kids | 0.211 ± | 0.520 ± | 2.47 | 5.66E−02 ± | 1.70E−01 ± |
| sunscreen | 0.005 | 0.011 | | 1.01E−02 | 9.20E−03 |
| Old Spice | 0.195 ± | 0.425 ± | 2.18 | 5.10E−03 ± | 1.62E−02 ± |
| anti- | 0.004 | 0.008 | | 7.32E−03 | 4.40E−03 |
| perspirant | | | | | |
| Mitchum | 0.187 ± | 0.339 ± | 1.82 | 2.47E−03 ± | 7.62E−03 ± |
| deodorant | 0.005 | 0.008 | | 6.72E−03 | 2.98E−03 |
| Banana | 0.151 ± | 0.229 ± | 1.52 | −1.60E−03 ± | −7.49E−03 ± |
| sunscreen | 0.005 | 0.008 | | 6.28E−03 | 2.30E−03 |
| Banana kids | 0.185 ± | 0.399 ± | 2.15 | 1.95E−02 ± | 6.47E−02 ± |
| sunscreen | 0.005 | 0.006 | | 6.82E−03 | 4.34E−03 |
| AW | 0.220 ± | 0.366 ± | 1.67 | 4.27E−03 ± | 3.12E−02 ± |
| toothpaste | 0.006 | 0.008 | | 7.17E−03 | 4.77E−03 |
| sunflower | 0.133 ± | 0.185 ± | 1.39 | 2.44E−04 ± | 5.11E−04 ± |
| oil | 0.003 | 0.003 | | 5.76E−03 | 3.06E−03 |
| Nutella | 0.139 ± | 0.269 ± | 1.94 | 3.55E−03 ± | 4.01E−02 ± |
| | 0.013 | 0.025 | | 5.90E−03 | 4.92E−03 |
| water | 0.139 ± | 0.213 ± | 1.53 | 6.36E−05 ± | −1.08E−03 ± |
| | 0.002 | 0.004 | | 5.77E−03 | 2.08E−03 |
| flour | 0.121 ± | 0.176 ± | 1.46 | 6.74E−03 ± | 7.03E−02 ± |
| | 0.001 | 0.002 | | 6.55E−03 | 7.07E−03 |
| powdered | 0.093 ± | 0.135 ± | 1.45 | 1.44E−02 ± | 1.28E−01 ± |
| sugar | 0.003 | 0.004 | | 5.84E−03 | 5.23E−03 |
| Material | 0.184 ± | 0.289 ± | 1.57 | 2.59E−03 ± | 3.05E−02 ± |
| A_6 | 0.018 | 0.005 | | 6.57E−03 | 4.01E−03 |
| Material | 0.186 ± | 0.280 ± | 1.51 | 4.02E−03 ± | 2.92E−02 ± |
| A_7 | 0.003 | 0.004 | | 5.64E−03 | 3.79E−03 |
| Material | 0.193 ± | 0.293 ± | 1.52 | 3.84E−03 ± | 3.14E−02 ± |
| A_5 | 0.003 | 0.005 | | 6.18E−03 | 3.98E−03 |
| Material | 0.131 ± | 0.191 ± | 1.46 | 1.05E−03 ± | 2.44E−02 ± |
| B_9 | 0.010 | 0.007 | | 7.08E−03 | 7.07E−03 |
| Material | 0.135 ± | 0.196 ± | 1.45 | −5.38E−04 ± | 2.94E−02 ± |
| B_121-1A | 0.004 | 0.006 | | 6.02E−03 | 7.36E−03 |
| Material | 0.113 ± | 0.169 ± | 1.50 | 1.73E−02 ± | 1.15E−01 ± |
| C_11 | 0.002 | 0.003 | | 6.54E−03 | 7.03E−03 |
| Material | 0.067 ± | 0.098 ± | 1.46 | 1.62E−02 ± | 1.19E−01 ± |
| C_14 | 0.002 | 0.004 | | 4.80E−03 | 4.94E−03 |
| Material | 0.066 ± | 0.096 ± | 1.45 | 1.30E−02 ± | 1.16E−01 ± |
| C_13 | 0.002 | 0.005 | | 5.03E−03 | 5.52E−03 |
| Material | 0.110 ± | 0.165 ± | 1.50 | 1.36E−02 ± | 1.15E−01 ± |
| C_10 | 0.002 | 0.005 | | 8.81E−03 | 7.96E−03 |
| Material | 0.113 ± | 0.167 ± | 1.49 | 1.49E−02 ± | 1.14E−01 ± |
| C_12 | 0.002 | 0.003 | | 5.80E−03 | 7.25E−03 |
| Material | 0.105 ± | 0.155 ± | 1..48 | 3.19E−03 ± | 3.92E−02 ± |
| C_18 | 0.001 | 0.003 | | 8.05E−03 | 1.17E−02 |
| Material | 0.119 ± | 0.177 ± | 1.49 | 1.77E−02 ± | 1.52E−01 ± |
| C_19A | 0.001 | 0.001 | | 1.03E−02 | 8.06E−03 |
| Material | 0.151 ± | 0.219 ± | 1.45 | 9.33E−04 ± | 3.66E−03 ± |
| C_7A | 0.011 | 0.021 | | 7.80E−03 | 7.30E−03 |
| Material | 0.127 ± | 0.178 ± | 1.41 | 1.35E−03 ± | 1.91E−02 ± |
| D_2 | 0.018 | 0.004 | | 6.99E−03 | 9.51E−03 |
| Material | 0.120 ± | 0.179 ± | 1.49 | 1.89E−03 ± | 2.05E−02 ± |
| D_1 | 0.002 | 0.004 | | 6.70E−03 | 1.04E−02 |
| Material | 0.120 ± | 0.180 ± | 1.51 | 1.95E−03 ± | 1.59E−02 ± |
| D_4 | 0.003 | 0.005 | | 6.53E−03 | 9.95E−03 |
| Material | 0.121 ± | 0.182 ± | 1.51 | 1.39E−03 ± | 1.90E−02 ± |
| D_3 | 0.003 | 0.005 | | 6.70E−03 | 1.00E−02 |

Once data were acquired, the absorption/refraction/scatter images were extracted and the scatter image corrected for beam hardening. A region of interest was chosen, typically including most of the material, and mean and standard deviation values were extracted for both attenuation (I/I₀) and scatter (V/V₀). The attenuation was converted into an attenuation coefficient μ=−ln(I/I₀)/t, where t represents measured sample thickness, and μ is in units of mean free attenuation paths per cm (cm⁻¹). In analogous fashion, we extracted a scatter coefficient v=−ln(V/V₀)/t, in units of mean free scatter paths per cm (cm⁻¹). Both quantities are implicitly weighted averages over all energies present in the spectrum. The variations observed in both absorption and scatter images were propagated as errors to obtain variation estimates for both quantities. This was performed for both the high and low energy spectra, and the quantitative results are shown in Table I: attenuation coefficient μ at low and high energy, and scatter coefficient v at low and high energy, for each sample measured.

The attenuation coefficients extracted from the phase contrast measurements can be interpreted in the same manner as conventional dual energy measurements, which are analyzed to estimate effective atomic number Z_{eff} and density ρ. Here, we examined the ratio of the low energy and high energy attenuation coefficients (μ_{L}/H); for a fully calibrated system this quantity can be related to the effective atomic number Z_{eff}. In FIG. 15 we plot μ_{L}/μ_{H} as a function of μ_{H}, which can be mapped approximately to density. Error bars indicate the variation observed within each sample.

Benign materials were shown in black. Water was indicated at μ_{H}=0.139 and μ_{L}/μ_{H}=1.53; a number of other benign materials had Z_{eff} similar to water, but at higher densities many of the benign materials also exhibited higher Z_{eff}. Threat materials were labeled by letters; with each letter signifying a single material, but samples within each group may have different preparation conditions. Material A showed a Z_{eff} similar to water but the density is significantly larger. Materials B and D were close to water in density and Z_{eff}, although slightly lower in both. Material C exhibited a wide range of densities corresponding to different preparation conditions, but Z_{eff} still close to that of water. In all four material categories, at least some of the samples exhibited density/Z_{eff} which were consistent with benign materials.

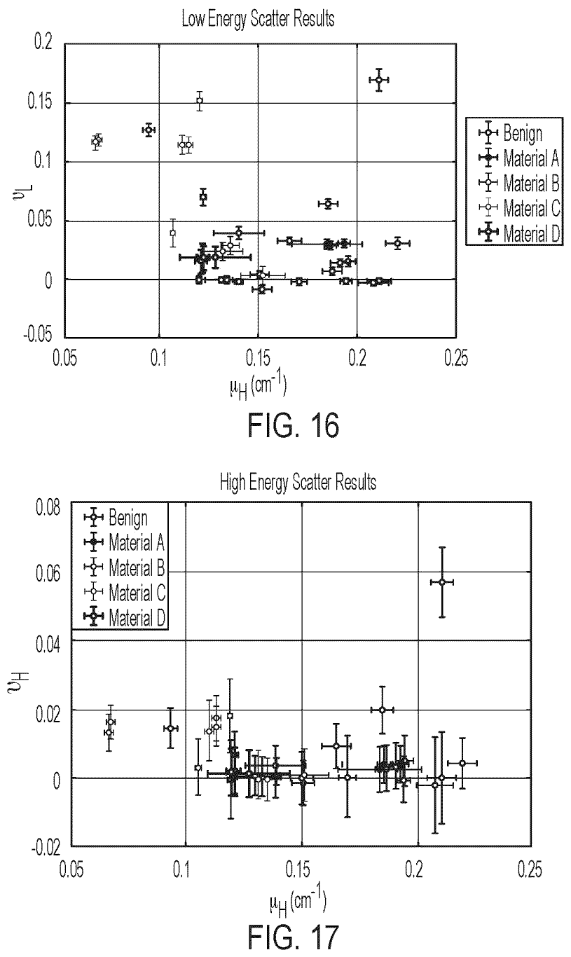

Next, we examined material properties revealed by scatter, plotting the scatter coefficient for the lower energy spectrum, v_{L}, as a function of density (approximated as μ_{H}), shown in FIG. 16. Note that a scatter coefficient of zero indicates no scatter was detected from a given material. The first qualitative observation we can make is that this plot is distinct from the dual energy information, with the distribution of properties clearly different than the previous plot, indicating that unique information is being displayed. For Materials A, B, and D, a small but significant amount of scattering is present. Note that in most cases, this distinguishes them from materials with otherwise similar density and atomic number. Material C covers not only a wide range of densities, but also a wide range of textures, from apparently homogeneous to very highly scattering, depending on the preparation method used.

The range of scatter values in the benign materials can be helpful for discrimination as well. At low density, powdered sugar (μ_{H}=0.093, v_{L}=0.13) exhibits very high scattering; flour is also fairly highly scattering (μ_{H}=0.12, v_{L}=0.07). Nutella® exhibits a moderate amount of scattering (μ_{H}=0.14, v_{L}=0.04). There were four different types of sunscreen, which illustrate an interesting range of scattering properties. One of the sunscreens, Banana Boat® (μ_{H}=0.15, v_{L}=−0.007), is an organic sunscreen and contains no metals; it is relatively low in density and no significant scatter is observed. Olay® sunscreen (μ_{H}=0.17, v_{L}=0.033) contains 3% ZnO particles and exhibits some scatter. Banana Boat Kids® contains 6% TiO₂ and 4% ZnO and shows higher scatter yet (μ_{H}=0.19, v_{L}=0.065). The final sunscreen (Badger® brand kids sunscreen) shows high Z_{eff}, high density, and high scatter (μ_{H}=0.21, v_{L}=0.17); it includes 19% ZnO particles, nearly as high of a concentration as our scatter step wedges. Other materials which show a small amount of scatter include deodorants and toothpaste, as can be seen in Table I. Note that materials which are homogeneous, such as water, sunflower oil, honey, and Vaseline®, display scatter values consistent with zero, confirming that the beam hardening correction process is successfully accounting for fringe visibility changes associated with spectral changes.

For the higher energy spectrum, the absolute values of all the scatter coefficients are reduced, as shown in FIG. 17. Several of the more weakly scattering samples cannot be distinguished from non-scattering materials, but the more strongly scattering materials are still distinguishable. Detailed numerical results can be seen in Table I.

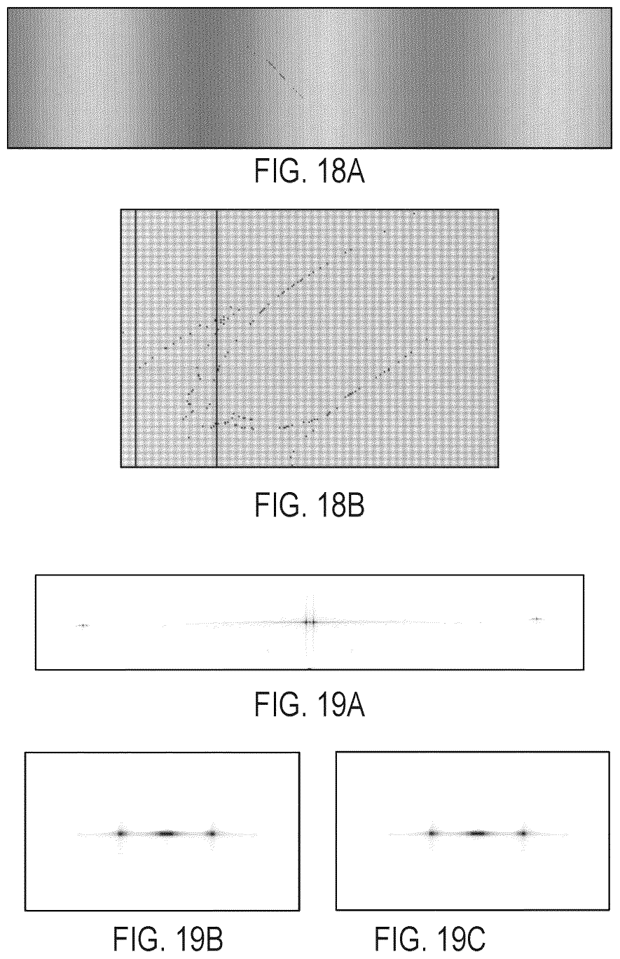

Often, not all pixels on the detector will record x-rays. Scratches or other damage can leave patterns of dead pixels. If not corrected, or if corrected using linear interpolation which is typically used for x-ray imaging, this can introduce artifacts into the reconstructed scatter and phase contrast images. The grating is typically aligned with the detector array, producing a pattern of vertical stripes. The grating period is typically chosen so that its period on the image is a few pixels. The image may show vertical intensity oscillations with a longer period, which might be a Moire pattern between the grating shadows and the pixel boundaries. The value of any given pixel (x, y) of the grating image will be designated g(x, y), and the image size will be Nₓ× N_{y} pixels. FIG. 18A includes an image of a region of the grating image, showing the vertical grating lines and the longer period Moire pattern. Near the center is a line of bad pixels from a scratch on the detector. FIG. 18B is a grating image with both the object and detector gratings in a section of bad pixels.

FIGS. 19A-19C include Fourier transforms of grating images. In FIG. 19A, a section of the Fourier transform of the grating image shows the central peak at (0, 0) and the first order harmonic peaks on either side at (±613, ±9). The non-zero y values of the first harmonic peak indicate that the grating was not perfectly aligned with the detector in this image. In FIG. 19B, a close-up of a region of the center of the Fourier Transform of the grating image shows the central peak at (0, 0) and Moire peaks at (±10, 0). In FIG. 19C, a close-up of the region around the first harmonic shows the harmonic peak at (613, −9) and convolutional Moire peaks at (603, −9) and (623, −9).

The Fourier transform of the grating image shows peaks at the origin and near the x-axis representing the Moire period, the grating period, and at intervals of the Moire period on either side of the grating period. If the grating period is long enough, higher harmonics of the grating will show up although typically the grating spacing and placement are chosen so that only the first harmonic appears. If a detector grid is present, peaks will be present at the detector grid period and its harmonics, as well as cross-harmonics between the detector and object grid. The grating Fourier transform will be denoted G(x, y).

After subtracting the dark image, bad pixels typically have values near zero. The grating image, despite its features, is usually relatively low contrast. A simple threshold cut-off on the grating image is usually effective for selecting pixels to fix.

The bad pixel detection can be made even better by reducing major sources of large period variation within the grating image. A copy G′ (x, y) is made of the Fourier transform of the grating image, and the regions around the central peak and the grating harmonic peak pairs are removed, making sure to include the Moire satellites around the major peaks. Let p_{M} be the Moire period in the grating image, (pₒₓ, p_{oy}) be the location of the first harmonic peak of the object grid, and (p_{dx}, p_{dy}) be the first harmonic peak of the detector grid. An acceptable filter is to choose r₁=2 μm and r₂=10 μm, and then use:

$$r ( x , y ) = \sqrt{{x}^{2} + {y}^{2}} \qquad ( 1 )$$

$$F ( x , y ) = \{ \begin{matrix}1 & r ( x , y ) > {r}_{2} \\ 0 & r ( x , y ) > {r}_{1} \\ \frac{1}{2} [ 1 - cos ( π ( r ( x , y ) - {r}_{1} ) / ( {r}_{2} - {r}_{1} ) ) ] & otherwise\end{matrix} \qquad ( 2 )$$

$${G}^{′} ( x , y ) = G ( x , y ) {\prod }_{j = - {n}_{o}}^{{n}_{o}} {\prod }_{k = - {n}_{d}}^{{n}_{d}} F ( x + {jp}_{ox} + {kp}_{dz} , y + {jp}_{oy} + {kp}_{dy} ) \qquad ( 3 )$$

where nₒ is the number of harmonic peaks of the object grid and n_{d} is the number of harmonic peaks of the detector grid.

This is then Fourier transformed back to give the image go (x, y). The bad pixels, being isolated aperiodic features, are composed primarily of high frequency components so the inverse Fourier transform preserves these structures. Since the zero-period component has been removed, the average value of the image will be zero. With most structure removed, almost all pixels will have values near zero while the dead pixels will have highly negative values. All pixels with values lower than a threshold value will be considered bad and removed. A reasonable threshold is −G(0, 0)/3.

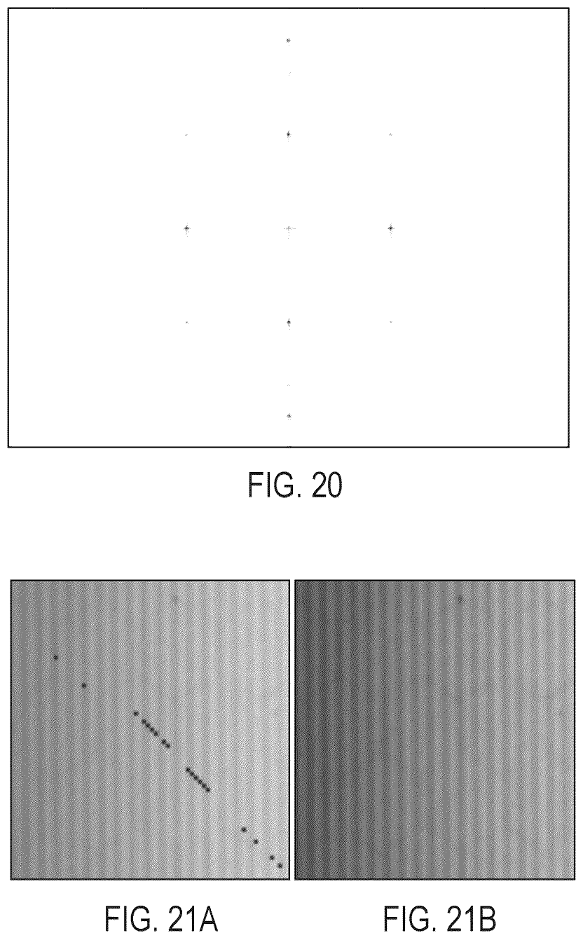

Because Fourier analysis based on convolutional patterns around the harmonic peaks are used for producing the scatter and phase contrast images, simply replacing a bad pixel by the average of its neighbors is insufficient. This neglects the short scale variation on the order of the grid pattern that is crucial to the analysis. Instead, we will use the idea that in the vicinity of any pixel out to a radius of a few pixels, the pixel values can be approximated by contributions from all harmonic and cross-harmonic peaks from the gratings. FIG. 20 includes a Fourier transform of a grating image with both an object and detector grid, showing first and second harmonics as well as cross-harmonics.

$$g ( x , y ) \approx {\sum }_{j = - {n}_{o}}^{{n}_{o}} {\sum }_{k = - {n}_{d}}^{{n}_{d}} {b}_{( jk )} {e}^{2 π i ( ( {jp}_{ox} + {kp}_{dx} ) x / {N}_{x} + ( {jp}_{oy} + {kp}_{dy} ) y / {N}_{y} )} . \qquad ( 4 )$$

The b₍ⱼₖ₎ are complex fitting parameters. Because g(x, y) is real, b₍₀₀₎ must be real and b₍₋ⱼ₋ₖ₎=b*₍ⱼₖ₎. However, there is no need to include these constraints in the algorithm, since unconstrained linear least squares fitting via singular value decomposition (SVD) is robust, simple, and reliable. Since this fit extends to several pixels around the bad pixel, detailed features within this region can be washed out for the fit. However, the image analysis technique involves a low-pass filter so that these details will be lost anyway. Despite the matrix-like terminology, conceptually in the fitting process b is a vector and the pair (jk) is treated as a single index. This can be accomplished with a mapping of (jk) to an index 1 running from 0 to 1 max=(1+2nₒ)(1+²n_{d}).

Choose a starting fitting step r_{f}, as the number of pixels in x and y to either side of the bad pixel to include in the fit. This should be chosen so that 1+2r_{f} at least covers one grating period. In addition, if entire columns of bad pixels are expected, it should be at least 2 to avoid ill-conditioned fits. For any given bad pixel at (x_{b}, y_{b}), scan over all pixels (x, y) such that x_{b}−r_{f}≤x≤x_{b}+r_{f} and y_{b}−r_{f}≤y≤y_{b}+r_{f}. If the n^{th} scanned pixel (starting from n=0) is not bad, add its value g(xₙ, yₙ) to the vector h of nearby good pixel value

$${h}_{i} = g ( {x}_{n} , {y}_{n} ) \qquad ( 5 )$$

and add a row to the matrix of fitting vectors A

$${A}_{i , l} = {e}^{2 π i ( ( {jp}_{ox} + {kp}_{dx} ) {x}_{n} / {N}_{x} + ( {jp}_{oy} + {kp}_{dy} ) {y}_{n} / {N}_{y} )} . \qquad ( 6 )$$

Let h have m elements, so that A is m×1 max in size. If m<lₘₐₓ, the fit will be ill-conditioned—there will be more fit variables bⱼ than constraints hᵢ. Even m=1 max is likely to lead to poor results. A reasonable criterion is to have the problem over-determined by a factor of 2. If m<6, increase r_{f} by 1 and find h and A again; repeat until m≥2 lₘₐₓ.

The problem is now a complex linear fit, h=A·b. This is solved by finding the SVD of A.

$$A = U \cdot W \cdot V$$

where V is a lₘₐₓ×lₘₐₓ unitary matrix, W is a lₘₐₓ×lₘₐₓ non-negative real diagonal matrix, and U is a m×lₘₐₓ matrix which is column orthonormal

$${u}_{j} \cdot {u}_{k} = {δ}_{jk} ,$$

uⱼ is the j^{th} column of U, and the symbol · indicates the inner product. The elements on the diagonal of W are called the singular values. The condition number of A is the ratio of the largest to the smallest of the singular values. If the condition number of W is more than 10, increase r_{f} by 1 and find h and A again.

Let W⁻¹ be the pseudo-inverse of W; a lₘₐₓ×lₘₐₓ diagonal matrix such that

$${\tilde{W}}_{ii}^{- 1} = \{ \begin{matrix}0 & if {W}_{ii} = 0 \\ 1 / {W}_{ii} & otherwise\end{matrix}$$

The fit vector is then found by

$$b = V \cdot {\tilde{W}}^{- 1} \cdot {U}^{†} \cdot h .$$

(It is worth noting that since we demand the condition number is finite, all Wᵢᵢ will be non-zero so that W⁻¹=W⁻¹.) Finally, set

$$g ( {x}_{b} , {y}_{b} ) = {\sum }_{l = 0}^{{l}_{max}} {b}_{ie} 2 π i ( ( {jp}_{ox} + {kp}_{dx} ) {x}_{b} / {N}_{x} + ( {jp}_{oy} + {kp}_{dy} ) {y}_{b} / {N}_{𝒴} )$$

Then repeat the procedure where h is filled with pixel values from the grating+object image, replacing the bad pixel in the grating+object image with the fitted value (A does not change between the grating and grating+object image, so it can be re-used and its SVD does not have to be re-computed).

If the grating absorption modulation is low compared to the average value of the image, the cross-harmonic peaks can be neglected. For images with a detector grid, a fairly long grating period (resulting in multiple harmonic peaks) in one or both grids, and many bad pixels, this can potentially result in significant time savings due to the O(mlₘₐₓ²) scaling of SVD.

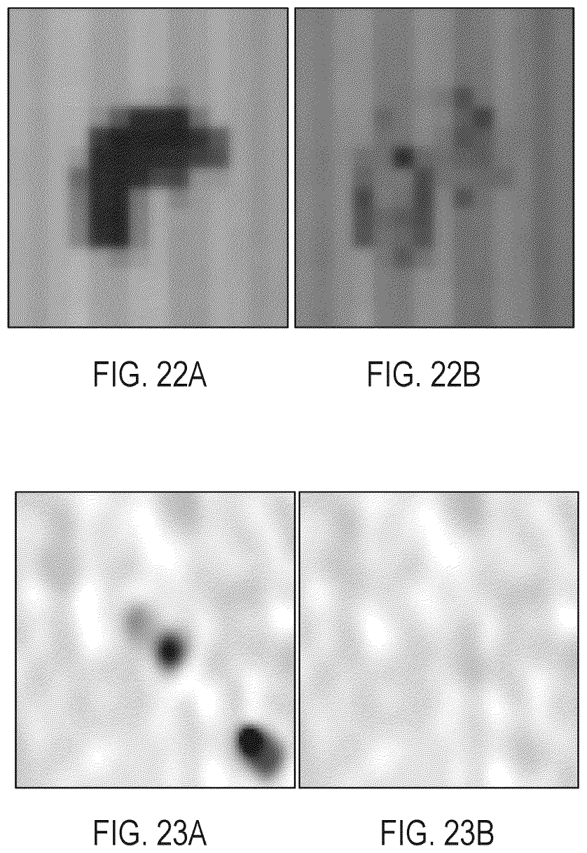

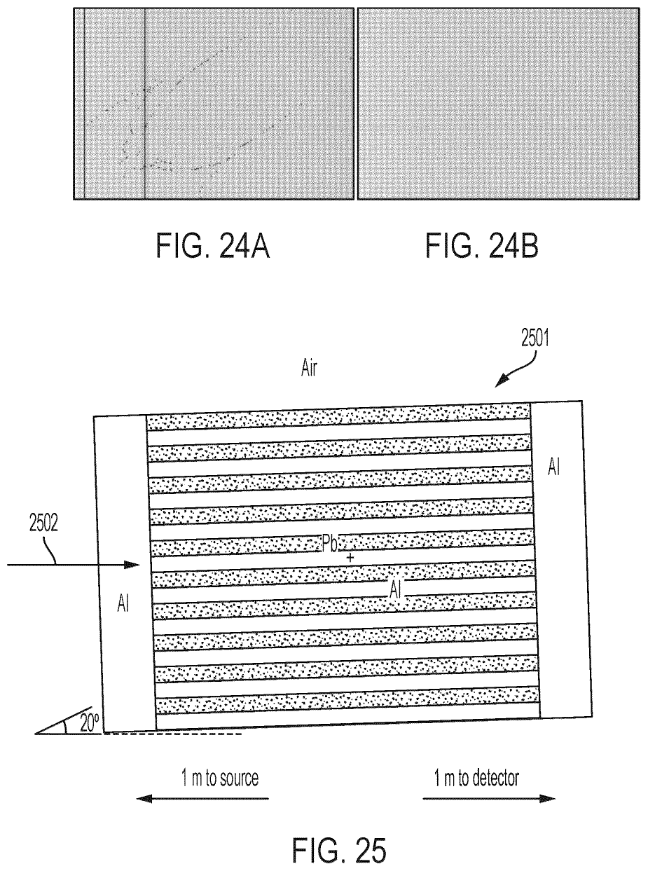

FIGS. 21 and 22 give examples of the bad pixel detection (FIGS. 21A and 22A) and correction algorithm (FIGS. 21B and 22B). FIGS. 23 and 24 show how fixing bad pixels improves the reconstructed phase and scatter images (FIGS. 23A and 24A are before correction, FIGS. 23B and 24B are after correction), respectively. FIG. 25 is an example of bad pixel correction with both an object and detector grating and with contributions from multiple harmonics from both gratings (FIG. 25B is after bad pixel correction).

In some embodiments, the signal-to-noise ratio in the acquired data can be significantly improved by tilting the source grating, the object grating, the detector grating, or combinations thereof. Tilting can effectively make line sources narrower without the burden of physically manufacturing gratings with extremely narrow grating elements (i.e. parallel channels with high aspect ratios). Tilting can comprise rotating the gratings about an axis parallel to grating element lines. In certain embodiments wherein the grating elements are parallel channels (each channel having a width and a height), the amount of tilt is greater than zero degrees and less than or equal to a maximum angle equivalent to the arctangent of the width of the channel divided by the height.

Referring to FIG. 25, a schematic illustration depicts a grating 2501 having 210 parallel lines per inch and utilizing a 2 degree tilt relative to an incident beam 2502 from an x-ray source. Experimentally observed visibility changes with grid tilt were simulated and the simulation results are discussed herein. The geometry and materials of the measurement conditions were set up in a transport model to calculate the total flux at the detector for various grid tilt (i.e., rotation) angles. The flux in an array of 48 μm pixels is tallied with MCNP 6.1—a Monte Carlo transport code to determine the pixel-based amplitude to average flux ratio. While the physics of the SAXS signal induced by a test object is not included in the purely atomic number-atomic mass (ZAID) based cross section libraries, the no-object visibility (to undergo reduction from object scatter) can be computed. Ideally this quantity should be as large as is obtainable for the greatest sensitivity.

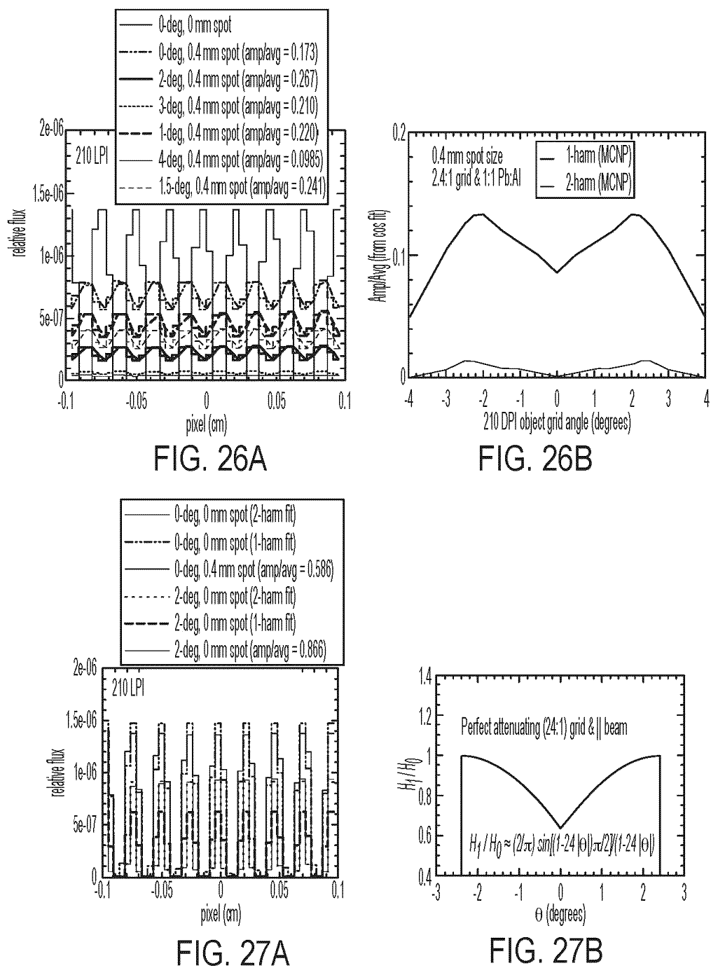

FIGS. 26A and 26B show modeled flux at the detector from a 0.4 mm spot size 100 kVp source showing values of fringe visibility fits (26A), and plot of fringe visibility (H₁/H₀) along with the second harmonic ratio (H₂/H₀) as a function of rotation angle (27B). FIG. 26A shows the modeled flux at the detector for various rotation angles. The grid visibility for each angle is determined from a fit to the first two harmonics and is plotted in FIG. 26B. As is in the measurement, a local minimum in visibility appears at zero degrees and increases symmetrically for angular changes from zero with a maximum value just before the fully transmitted grid openings go to zero.

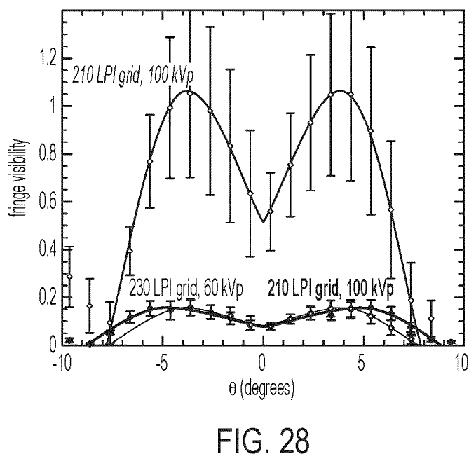

This behavior for x-rays emitted from a 0.4 mm spot at 2 m from the detector is very unexpected. A hint at its underlying origin is revealed by considering an idealized signal from a 0 mm spot (FIG. 27A) or even more strikingly from an idealized parallel beam (FIG. 27B). FIG. 28B shows that an obvious consequence of grid rotation is an effective reduction of transmitted duty-cycle. While a reduction of the fringe amplitude or first harmonic can be seen for increasing |θ| (smaller effective duty-cycle), the fringe average is reduced by an even greater amount resulting in increased ratio visibility and thus increased scatter sensitivity. Additionally, in the limit of parallel beam and perfect attenuation of the lead, an analytical relationship for harmonic ratios is simply

$${( \frac{{H}_{n}}{{H}_{0}} )}_{‖} = \frac{P}{π ns} sin \frac{πns}{P}$$

where s is the effective slit width for the rotated grid. Assuming perfect attenuation for the lead absorber regions the effective slit width as a function of rotation angle is given by

$$\frac{s}{P} = \frac{1}{2} - \frac{T}{2 {s}_{1 : 1}} \vert θ \vert ,$$

where P is the grid period, T is the grid thickness, s 1:1 is the width of the transmitting slits for the 1:1 duty-cycle grid and θ is the “small” rotation angle. Also, the effective duty-cycle (d_{c}:1) can then be expressed with

$${d}_{c} = \frac{s}{P - s} .$$

In the parallel beam case it is interesting to note that the first harmonic ratio is a maximum when both s/P and the intensity go to zero.

This behavior is relaxed by the more appropriate non-parallel beam and finite spot size physics. The s/P equation above still assumes perfect attenuation, but not a parallel beam and can be used as an approximate mapping from simulated rotation to simulated duty-cycle. A consequence of this mapping is that the analytical relationship for harmonic ratios above can be empirically modified to give a reasonably accurate fit to the non-parallel beam data with

$$\frac{{H}_{1}}{{H}_{0}} = {a}_{0} \frac{sin [ {a}_{3} ( 1 - {a}_{1} \vert θ \vert + {a}_{2} {θ}^{2} ) ]}{1 - {a}_{1} \vert θ \vert + {a}_{2} {θ}^{2}}$$

where aⱼ are four fit parameters. Interpreting a₁ as T/s_{1:1} and given θ for the maximum H₁/H₀, the equations for s/P and for d_{c} can be used to infer the grid duty-cycle having maximum sensitivity. FIG. 28 shows that the modified harmonic ratio gives very reasonable fits to rotated object grid measurements indicating that the effect is due to a change in duty-cycle even when the source deviates from a parallel beam. Using the modified harmonic ratio fits, the maximum fringe visibility gives an approximate effective duty-cycle 1:7 for both the 210 LPI grids and 1:4 for the 230 LPI grid.

### Additional Cross-Grid Examples

While radiography is an important tool for inspection of objects in many settings, it provides little material information. Phase contrast x-ray imaging provides additional material information compared with commonly used x-ray imaging. With phase contrast x-ray imaging, three images can be obtained: absorption (similar to commonly used x-ray imaging), differential phase, and dark field. Cross-grid Moire imaging adapts phase contrast imaging to commonly used x-ray sources with a broad spectrum and possibly large focal spot, and detectors which may have a large field of view but low spatial resolution, while providing a way of distinguishing sample microstructure from the effects of beam hardening. This can be especially beneficial for non-scanning inspection applications involving larger objects such as industrial non-destructive evaluation and use by first responders.

Phase contrast, or multi-contrast x-ray imaging relies on patterning the x-ray beam (using an “object grid”) to detect new signatures based on not only attenuation but also directional changes of the x-rays in an object. Herein, “grid” can be used to refer to a grating. In exampled described hereinabove, methods of performing this measurement use penetrating x-rays along with an additional optic such as a detector grid to correct for spectral effects and enable quantitative interpretation of the signal. In some described examples, a high resolution detector can be used to directly image the projections of the object grid and detector grid, with the detector grid rotated 90 degrees relative to the object grid. However, this may not be feasible for use with large field of view detectors, which have larger pixels and cannot resolve the lines. In further described examples, indirect detection may be used, e.g., where an analyzer grid is used to form Moire fringes with the object and detector grid regions, but in these examples the object and detector grids cover different locations and the object is scanned across.

In accordance with additional examples, methods involve indirect imaging of crossed object and detector grids, e.g., by using a crosshatched analyzer grid that can be translated in two dimensions. As will be discussed further below, this can enable the use of fine patterning elements with a large detector and a static object.

The crossed analyzer grid forms moiré fringes with both the object and detector grids. By translating the analyzer grid both horizontally and vertically, a series of measurements of the moiré fringes can be made at different phases of the object and detector grid. Translating both directions allows indirect measurements of both patterns, but doing so at each combination can ensure that the patterns are well sampled with different projected spectra. This can allow for the use of arbitrarily fine optics, enabling high sensitivity measurements, with arbitrarily large detectors. This can be important for many applications such as non-destructive examination, medical imaging, and emergency response.

Disclosed additional cross-grid examples relate to the examples described hereinabove (e.g., U.S. application Ser. No. 16/363,989, now issued as U.S. Pat. No. 11,006,912) and Moird scanning examples involving indirect imaging of a moving object described in Ser. No. 17/322,635 (now issued as U.S. Pat. No. 11,639,903 and incorporated by reference herein). While others have used calibration objects to correct for beam hardening influencing a dark field image, that tends to be most effective when the range of materials being inspected is limited. Further other techniques and approaches do not make this type of correction and cannot directly distinguish spectral effects from structural effects when viewing a dark field image. With phase contrast an x-ray beam can be patterned and a detector can be spaced further along the beam axis so that distortions in that pattern can be detected. The detected image can provide information regarding how the x-rays change direction, due to refraction or small angle x-ray scattering. Finer patterns provide the ability to detect smaller angular changes and higher sensitivity to texture and electron density gradients but may not be directly visible on a detector with low spatial resolution. With a large field of view but low spatial resolution detector, a fine pattern can be detected using an additional grating positioned near the detector that matches the projected pattern and then the detector grating can be translated and so that moiré fringes can be detected instead of the directly viewing the pattern.

As discussed above, it has been found that it can be desirable to use a loss of contrast in a projected pattern as a fingerprint for small-angle scattering. However, for x-ray sources with a broad spectrum of energies, it has been further found that objects can preferentially attenuate the more easily attenuated parts of the beam. This can result in the projected pattern having a lower contrast or reduced visibility because the spectrum that is being used to interrogate the object is now different. In Ser. No. 16/363,989, examples described the addition of a grating proximate the detector which would not be sensitive to angular changes given its positioning. This detector grating could then be used to detect the spectral changes and separate the changes from the spectral angular changes associated with the object grating that was positioned proximate the object. However, many of such arrangements correspond to direct imaging, with many being limited to situations in which the pattern is sufficiently large, or the field of view was sufficiently small to allow for direct imaging.

In additional cross-grid examples, radiography can be performed for emergency response, such as where an unknown or threatening object is observed in a public setting and its desirable to image the contents of the object, as well as for medical imaging and non-destructive evaluation. Such examples can use phase contrast to achieve indirect imaging to avoid the requirement of a large detector for direct imaging. For example, larger detectors have pixels that tend to be correspondingly bigger. If direct imaging is to be performed, then an available sensitivity to angular changes would be lower because the patterns used would be coarse. Thus, direct imaging can be impractical or produce images with insufficient resolution. Disclosed cross-grid examples can incorporate both object and detector gratings while leveraging indirect imaging with moiré fringes. In various disclosed examples, an arrangement of object and detector gratings included respective sets of elements that complement each other, e.g., by forming a crossed pattern. An analyzer grating having a pattern matching the projected pattern of the object and detector gratings (e.g., crossed) can then be positioned in the beam path and stepped in relation to the two object and detector grating patterns.

Movement of the analyzer grating (or object and detector gratings), e.g., with a coordinated translation to periodic positions associated with the elements of the projected pattern, can allow for the collection of a set of images that can produce improved images in the application of moiré indirect imaging techniques. Example movements are generally transverse to the direction of propagation of the x-rays, e.g., a beam axis. In many examples herein, transverse refers to perpendicular, approximately perpendicular (e.g., within 5 degrees), within 10 degrees, or within 30 degrees. With such movement, different pattern overlaps between the object, detector, and analyzer gratings can be obtained. For example, where an object grating contains vertical grating elements and a detector grating contains horizontal elements, movement of a crossed analyzer grating horizontally can provide information relating to the object grating, and movement of the crossed analyzer vertically can provide information relating the detector grating.

Accurate extraction of absorption, phase, and scatter information using cross-grid indirect moiré imaging techniques requires careful measurements and data analysis, or image artifacts may result. As will be discussed further, disclosed examples can reduce or eliminate many undesirable artifacts. For example, it can be seen that if the analyzer grating is moved along two separate directions, i.e., one line in one direction (vertical), one line in the other direction (horizontal), as the analyzer grating is moved in the horizontal direction the object and detector gratings are positioned at one particular condition in the vertical direction. Thus, the analyzer grating can be positioned to interrogate a particular voxel of material that is positioned on a horizontal stripe, and the analyzer grating can be translated horizontally to reveal information related to changes in the vertically oriented grating pattern. Errors in alignment can result in patterns which are not perpendicular can be addressed during data analysis, as can errors in phase stepping. Also, the patterns could be positioned on an attenuating portion of the patterns, a transmitting portion, or somewhere in between. Each can provide somewhat different information due to the complexity of the spectral portion of the image data and the fact that even though grating patterns may have orthogonal characteristics, the patterns are not necessarily uncoupled due to coupling through spectral changes that are occurring.

In selected examples, artifact reduction can be achieved with additional scan points being collected along more than a single vertical translation and a single horizontal translation of the analyzer grating. In some examples, a vertical, horizontal, and a diagonal set of scan positions can be used to collect images. In further examples, a more complete array of scan positions can be imaged (e.g., a 6×6 array, 9×9 array, etc.). That a benefit can be obtained from the additional scan positions is non-trivial, and it has been found that it can be used to effectively average spectral information in a way to represent the changes that are occurring with both object and detector grating patterns. As discussed further below, this can be used reduce artifacts in phase contrast images. Various scan paths can be used, such as a snake-like raster pattern, an L-shaped pattern, a triangular pattern, etc. Scan steps can have various amounts and can depend on the grating and projected pattern periodicities. In some examples, 40 micron steps are used though other steps can be suitable, such as 10 micron, 20 micron, 80 micron, 200 micron, etc. In many examples, at least three steps/period are used in scan sequences, though other periodic increments can be suitable (e.g., 4 steps/period, 6, 8, 12, etc.). Micron steps are typically a suitable order of magnitude, but it will be appreciated that other steps and increments can be suitable.

As stated, a 2-d pattern of scan positions can be applied to cross-grid pattern arrangements of object, detector, and analyzer gratings. By using a relative movement of the gratings to define the 2-d pattern, the analyzer grid can distinguish information relating to the object and detector gratings. Further, there can be a subtlety with respect to how the scanning is performed, e.g., by scanning in more than a set of a single horizontal scan line and a single vertical scan line. In comparison to other existing approaches, it should be noted that detector grids are not typically used for spectral corrections, with most avoiding spectral corrections or doing them empirically for only a single material. Thus, disclosed examples can uniquely perform moiré-based techniques by using object and detector gratings and an analyzer grating with a pattern related to the object and detector grating patterns.

Thus, disclosed additional cross-grid examples advantageously can be used for inspection of large objects, as referenced above, e.g., in industrial radiography, medical imaging, and emergency response. In combination with examples in U.S. application Ser. No. 16/363,989 described above (and related beam hardening techniques) and in U.S. application Ser. No. 17/322,635 in which methods of doing phase contrast imaging with penetrating x-rays are described, such additional examples can enable the application of phase contrast imaging for large objects. This can beneficially provide additional material information (density, effective atomic number, and microstructure) relative to conventional x-ray imaging. In contrast to techniques described in Ser. No. 17/322,635, many disclosed cross-grid examples can be applied to a static object. Further, while being able to inspect a static object using indirect imaging while also applying beam hardening correction, disclosed cross-grid examples can address the complexity that exists in which the object and detector gratings are both projecting over the whole image of the object. In other words, rather than separating the field of view into spaced-apart object and detector portions, the analyzer grating can be used (through movement of one or more of the gratings) to reveal information associated with both the object and detector gratings without requiring a movement of the object.

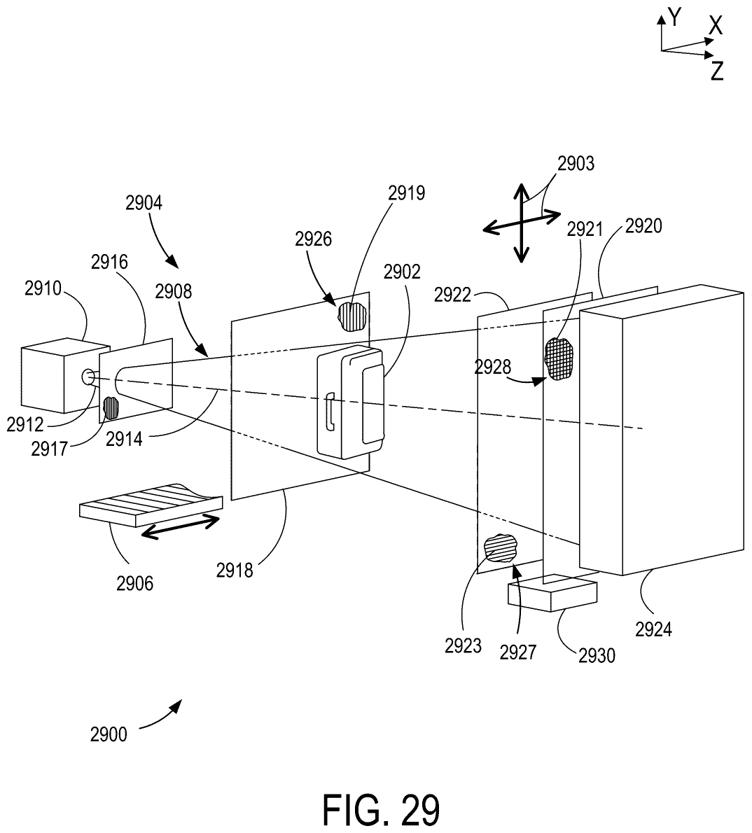

FIG. 29 is an example x-ray system 2900 that can produce indirect moiré images of an object 2902 rather than direct images. The object 2902 can be complete or whole (such as an entire suitcase, container, backpack, device, etc.) or can be a portion of a larger object. In representative examples, the object 2902 can be statically positioned in relation to an x-ray optical arrangement 2904, e.g., by moving the object 2902 with a conveyor 2906 or other movement mechanism into a field of view 2908 of the x-ray optical arrangement 2904, where it remains stationary during the measurement, or by placing the imaging equipment around a static object. The x-ray optical arrangement 2904 can include an x-ray source 2910 situated to produce and direct an x-ray beam 2912 along a beam axis 2914. The x-ray beam 2912 can be directed through a source grating 2916 situated in proximity to the x-ray source 2910. The source grating 2916 can be configured to break up the source x-rays of the x-ray beam 2912 into a plurality of spaced apart x-ray line sources with an arrangement of linear grid elements 2917 (shown vertically arranged in the figure). The line sources can be used to obtain increased resolution for projecting through an object grating 2918. In some examples of the system 2900, the source grating 2916 is not present, such as where the x-ray source 2910 produces spot sizes that are sufficiently small, as discussed previously hereinabove.

The object grating 2918 can be situated near the object 2902 along the beam axis 2914, such as immediately before or after the object 2902. In representative examples, the entirety of a region of interest of the object 2902 receives the x-ray beam 2912. For example, the region of interest can correspond to extend across the entire field of view 2908 or a portion of the field of view 2908. The x-ray beam 2912 continues to propagate through an analyzer grating 2920 and a detector grating 2922 before being detected with an x-ray detector 2924. The analyzer grating 2920 and detector grating 2922 can be situated near the detector 2924, typically with the analyzer grating 2920 situated immediately before the detector 2924 along the beam axis 2914 and the detector grating 2922 preceding the analyzer grating 2920, although the analyzer grating 2920 can also precede the detector grating 2922. In representative examples, the entirety of the x-ray beam 2912 that propagates through the object 2902 also propagates through the analyzer grating 2920 and detector grating 2922.

The object grating 2918 has a set of grating elements 2919 arranged in an object grating element pattern 2926 and the detector grating 2922 has a set of grating elements 2923 arranged in a detector grating element pattern 2927 which is different from the object grating element pattern 2926. In some examples, the object grating element pattern 2926 and the detector grating element pattern 2927 differ at least by or only by a rotation angle, typically 90 degrees. For example, in many arrangements, the grating elements 2919 can be a set of linear grating elements perpendicularly arranged with respect to the beam axis 2914 (e.g., vertical, or in the ‘y’ direction in FIG. 29) and the grating elements 2921 can be a set of linear grating elements also perpendicularly with respect to the beam axis 2914 but further perpendicularly arranged with respect to the grating elements 2919 (e.g., horizontal, or in the ‘x’ direction in FIG. 29).

The analyzer grating 2920 has a set of grating elements 2921 arranged in an analyzer grating element pattern 2928. In representative examples, the analyzer grating element pattern 2928 is configured to have significant correspondence to a projected image of the object grating element pattern 2926 and the detector grating element pattern 2927. Thus, in examples in which the linear grating elements 2919 define the object grating element pattern 2926 as a set of stripes extending in a first direction perpendicular to the beam axis 2914 and the linear grating elements 2923 define the detector grating element pattern 2927 as a set stripes extending in a second direction perpendicular to both the beam axis 2914 and the first direction, the analyzer grating element pattern 2928 can be arranged in a crossed-grid pattern. Such an analyzer grating can also be referred to as a crossed-line analyzer grid, crosshatched analyzer grid, etc. Perpendicular linear grating elements can be convenient because they are separable from each other, but other sets of elements defining respective patterns can be suitable provided they can be separable. Other separable patterns typically include some orthogonality characteristic, but other non-orthogonal patterns might be used provided they can be disentangled from each other, which in some examples can be done after detection through mathematical analysis. In general, patterns are separable where they are different from each other such that a grating movement can allow detection of changes associated with one pattern over the other.

The degree of significant correspondence results in a moiré pattern formed between the object grating and analyzer grating, as well as a moiré pattern formed by the detector grating and analyzer grating. One or more the gratings 2918, 2920, 2922 can be moved relative to the other(s) and multiple images can be collected to perform moiré phase contrast imaging, extracting absorption, differential phase, and dark field images. To achieve such movement, in many examples, the analyzer grating 2920 is translated in directions 2903 with a movement stage 2930 while the object and detector gratings 2918, 2922 remain fixed. The degree of significant correspondence need not be defined by a perfect overlap in every example, as such an overlap may be impractical to achieve. However, in many examples, such an enhanced correspondence can be advantageous. In general, the greater the correspondence between the object grating 2918 and analyzer grating 2920, or between the detector grating 2922 and analyzer grating 2920, the greater contrast will be present in the resulting moiré fringes, resulting in increased signal-to-noise when extracting absorption, differential phase, and dark field images. Where there is an insufficient matching between the patterns 2926, 2927 to the pattern 2928, beat frequencies can occur within a pixel with successive translations resulting moiré fringes which are too fine to detect, making it impossible to extract absorption, differential phase, and dark field images. By way of example, where the object and detector gratings having grating elements defining respective patterns of dots (i.e., rather than stripes or other patterns), the analyzer grating pattern (which contains an additive combination of both patterns) should be able to be moved across the projection of the object and detector grating patterns in order to have knowledge as to which set of dots is being sampled. In other words, the pattern from which phase information is being obtained should be able to be selectable, while at the same time the patterns should have the same or sufficiently similar response to a change in spectrum. For an object placed in the path of the beam 2912, the beam 2912 should affect the detected spectra in the same or similar way.

Object and detector gratings 2918, 2922 with orthogonally arranged lines (or stripes) as respective grating elements 2919, 2923 can be particularly suitable. Such a pattern set can afford an explicit direction to probe either set with an analyzer grating 2920 containing a crossed grid pattern of grating elements 2921. This probing is allowed because a relative movement of the aligned patterns 2926, 2927, 2928 can be provided, e.g., through movement of the analyzer grating 2920 with the movement stage 2930 along directions 2903, movement of the object and detector gratings 2918, 2922 to achieve a similar effect, or a combination of movements. In some examples, grating elements 2919, 2921, 2923 of one or more of the object, analyzer, and detector gratings 2918, 2920, 2922 are not continuous elements, e.g., they can be dashed, dots (square or with roundedness), or include other discontinuities or shape variations, such as periodic variations associated with any periodicity associated with the grating elements 2919, 2921, 2923. In general, differences between grating patterns amongst one or more of the gratings are sufficient to provide separability, such as to discern movement of one relative to another.

In some examples, enhancement in phase contrast moiré imaging can be achieved by collecting x-ray image data with the detector 2924 at multiple movement positions of the gratings 2918, 2920, 2922 relative to each other. In particular, it has been found that the varied movement positions result in different samples of the incident x-ray spectrum due to beam hardening, and improper averaging can result in undesirable artifacts. These artifacts can be removed from produced phase contrast images in part by translating to more positions than an “L” shaped set of scan positions of the analyzer grating 2920 relative to the object and detector gratings 2918, 2922. For example, the movement of gratings can be rastered or painted to collect a set of scan positions that extends to form a 2-d array of collected images. It has also been found that a significant benefit with respect to image sampling can be obtained without a full 2-d array, e.g., by collecting a set of images along an “L” shaped set of scan positions and a diagonal set of scan positions. The data collected from the additional scan positions allows for averaging over different spectra, and can be used to produce an improved phase contrast image in which artifacts are removed that would otherwise obscure attenuation characteristics in the image. Such characteristics can be associated with material specific identifications. Careful selection of scan and image collection positions can provide artifact reduction with the minimal number of images collected, reducing the duration of scanning and image collection processes, thereby enhancing time-critical image collection and analysis.

### 2-D Phase Scanning

As discussed above, analyzer gratings can include a combination of object and detector grating patterns and a relative movement between gratings can be used to produce moiré patterns and phase contrast images. For convenience in describing 2-D phase scanning techniques that can be used in accordance with additional cross-grid examples, an analyzer grating (e.g., grating 2920) that includes a 2-dimensional rectangular grid of holes can be used, though it will be appreciated that other patterns may be used as discussed herein.

The hole pattern of the analyzer grating can be used to phase scan moiré patterns of both an object grating and a perpendicularly arranged detector grating (e.g., object grating 2918 and detector grating 2922). The phase scan can occur in the same section of the field of view of a detector (e.g., detector 2924) without replacing any of the gratings. Automated phase steps can be performed on the analyzer grating with a motorized translation stage (e.g., movement stage 2930). Alternatively, the object and detector gratings may be translated or another translational arrangement may be provided.

At multiple positions provided by x- and y-translations of the grating and/or gratings, the x-ray beam can be directed through the object and gratings and x-ray phase scan images can be collected with the detector. Each of the collected images associated with the x- and y-direction translation step sequences can be analyzed. For example, the Fourier transforms (FT) can be taken of the phase scan images, and the peaks of the x- and y-analyzer grid patterns can be located on the FT image. The phase of the FT at the pixel corresponding to a peak can provide a measurement of the phase displacement of the analyzer grid relative to the object and detector grids. For each pixel in the untransformed images, the sequence of phase step images gives a sinusoidal pattern with measured intensity on the vertical axis and the phase of the step on the horizontal axis. The first two terms of the Fourier transform of this sequence gives the mean value, amplitude, and phase for the pixel. The contrast of the sequence for that pixel is the ratio of the amplitude to the mean. The absorption is the ratio of the mean values of an object image and a flatfield image; the phase shift is the difference in phase between the object and flatfield images; and the contrast reduction (related to the scatter) is the ratio of the contrasts of the object and flatfield images.

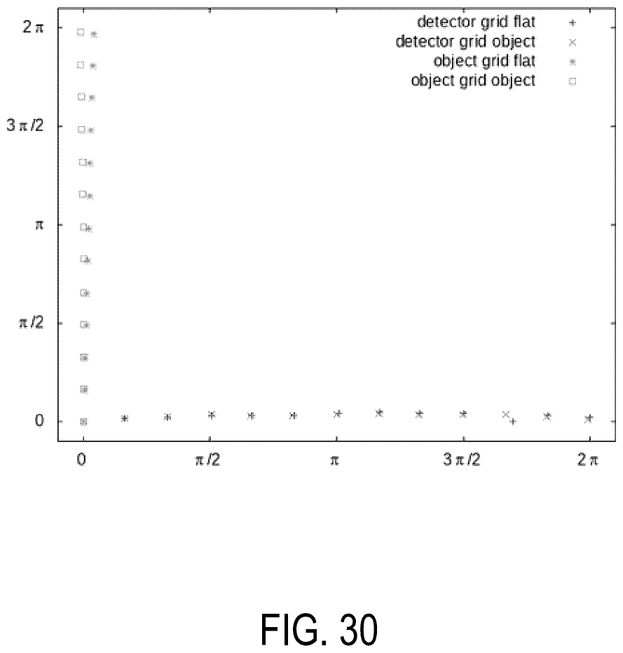

A cross-talk between the x- and y-stepping directions can be present. A stepping sequence along the x-direction, for example, might not be purely constant in y-, thereby allowing phase from the perpendicular grid to influence the signal intended to be measured. Such a cross-talk can be seen in FIG. 30, which shows horizontal and vertical phases during a scan and image collection at 13 positions in the x- and y-directions. Small excursions in the transverse direction are seen. While these are reproducible for the detector grating sequence for the object and flatfield image, a drift is present in the transverse direction phase for the object grating scan.

To correct for this, the horizontal phase can be denoted by and the vertical phase by v. Because only the lowest order terms (first and second) in the Fourier transform are generally of interest, every pixel (x, y) can be expressed as the following function of and V:

$$I ( x , y ; μ , v ) = M ( x , y ) + {A}_{d} ( x , y ) cos [ - μ - {ϕ}_{d} ( x , y ) ] + {A}_{o} ( x , y ) cos [ - v - {ϕ}_{o} ( x , y ) ]$$

with M the mean value of the pixel, A_{d}, the amplitude of the detector grid, Aₒ the amplitude of the object grid, P_{d} the detector grid phase shift, and ϕₒ the object grid phase shift. The minus signs in the cosine function are due to the code for determining the 1-D parameters being originally coded as:

$${\sum }_{i} {I}_{d , i} cos [ {μ}_{i} ]$$

(and similarly for the object grid), and so integrating over cos[μ] picks out the cos[−μ] Fourier component. From the 1-D Fourier transforms, we have a good first estimate of all five parameters. The phase step sequence with N steps in each direction gives 2N data points Iₘ(x, y; μ, v) at known (μ, v) as described earlier. This allows us to solve for the 2-D case including drift by minimizing:

$${χ}^{2} = {\sum }_{m = 1}^{2 N} {( {I}_{m} ( x , y ; μ , v ) - I ( x , y ; μ , v ) )}^{2} .$$

This can be achieved with a Levenberg-Marquardt minimizer algorithm.

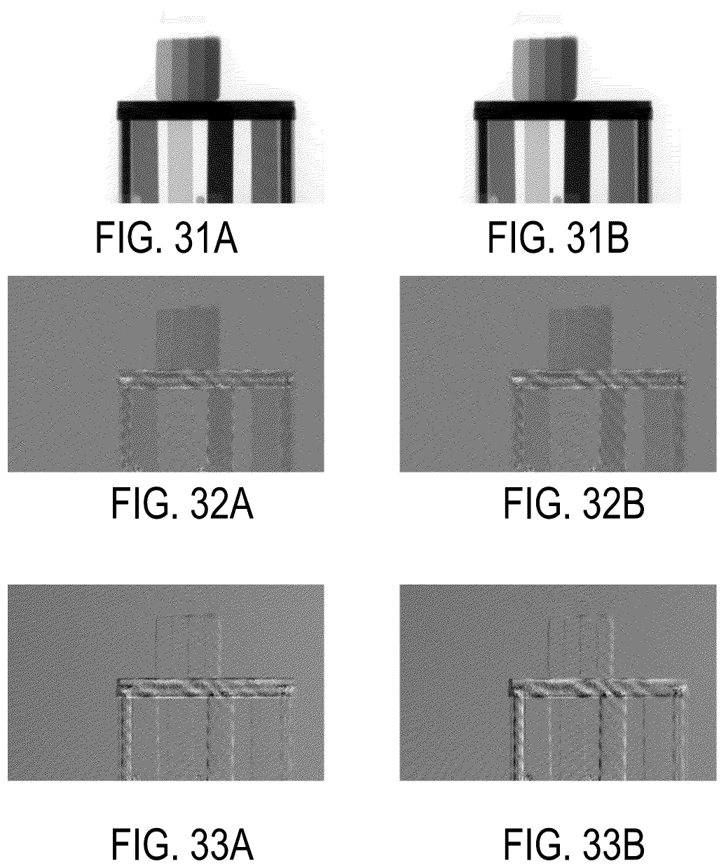

FIGS. 31A-33B show phase contrast images comparing an uncorrected and phase drift corrected images for a test object. More specifically, FIG. 31A shows an absorption image and FIG. 31B shows a drift corrected absorption image. FIG. 32A shows a contrast ratio image for the same image in FIG. 31A and FIG. 32B shows a contrast ratio image for the same drift corrected image from FIG. 31B. FIG. 33A shows a phase shift image for the same image in FIG. 31A and FIG. 33B shows a phase shift image for the same drift corrected image from FIG. 31B.

### Spectral Effects in Moiré Scanning

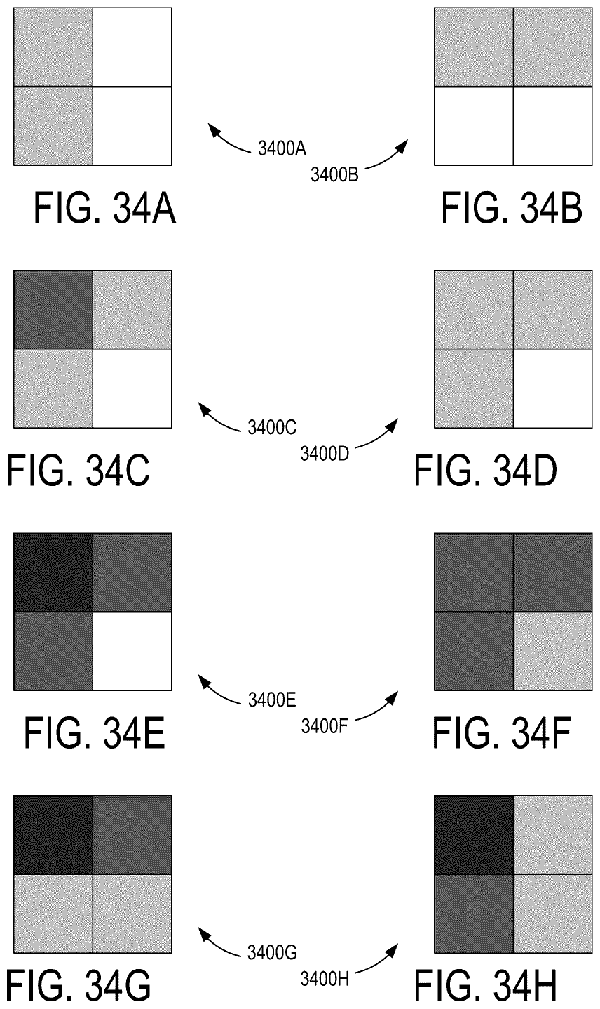

FIGS. 34A-34H show a set of grating unit cell representations for object, detector, and analyzer gratings having vertical, horizontal, and crossed grating elements, respectively. More particularly, FIG. 34A shows a tiled set defining a unit cell 3400A of an object grating having a repeating set of vertically arranging grating elements. Similarly, FIG. 34B shows a tiled set defining a unit cell 3400B of a detector grating having a repeating set of horizontally arranging grating elements. A shown in FIG. 34C, when the unit cells 3400A, 3400B are overlayed, a unit cell pattern 3400C is produced. Similarly, FIG. 34D shows a tiled set defining a unit cell pattern 3400D of an analyzer grating having a repeating set of vertical and horizontal elements. As can be seen, a geometry of the pattern 3400D matches a geometry of the pattern 3400C. While the analyzer grating can be shifted in any direction to arbitrarily position the open element in its lower right, it is useful to examine extreme positions. For the high part of the object and detector grating fringe, the white squares can be allowed to overlap, forming the unit cell 3400E shown in FIG. 34E. For the low parts of the fringes of both gratings, the unit cell 3400F in FIG. 34F is seen. This can be achieved by positioning the white square of the analyzer grating unit cell 3400D in the upper left. Where the detector grating is positioned high and the object grating is positioned low, the unit cell 3400G can be obtained, as shown in FIG. 34G. Where the detector grating is low and the object grating is high, the unit cell 3400H can be obtained, as shown in FIG. 34H. As shown, FIGS. 34A-34H show a specific simplified example in which each grating provides a uniform 50% attenuation.

A mathematical analysis of the spectral effects proceeds as follows. Integrated signals can be defined:

| | |
|---|---|
| Sₕₕ | Integrated signal of one cell in detector |
| | grid high, object grid high case |
| Sₗₕ | Integrated signal of one cell in detector |
| | grid low, object grid high case |
| Sₕₗ | Integrated signal of one cell in detector |
| | grid high, object grid low case |
| Sₗₗ | Integrated signal of one cell in detector |
| | grid low, object grid low case |

Visibilities can then be formed as:

| | |
|---|---|
| ${V}_{oh} = \frac{{S}_{hh} - {S}_{hl}}{{S}_{hh} + {S}_{hl}}$ | Object grid visibility in the high signal region of the detector grid fringe. |
| ${V}_{ol} = \frac{{S}_{lh} - {S}_{ll}}{{S}_{lh} + {S}_{ll}}$ | Object grid visibility in the low signal region of the detector grid fringe. |
| ${V}_{dh} = \frac{{S}_{hh} - {S}_{lh}}{{S}_{hh} + {S}_{lh}}$ | Detector grid visibility in the high signal region of the object grid fringe. |
| ${V}_{dl} = \frac{{S}_{hl} - {S}_{ll}}{{S}_{hl} + {S}_{ll}}$ | Detector grid visibility in the low signal region of the object grid fringe. |

For the case where an object is partially attenuating the beam:

| | |
|---|---|
| \tilde{S}ₕₕ | Integrated signal of one cell in detector |
| | grid high, object grid high, object present |
| \tilde{S}ₗₕ | Integrated signal of one cell in detector |
| | grid low, object grid high, object present |
| \tilde{S}ₕₗ | Integrated signal of one cell in detector |
| | grid high, object grid low, object present |
| \tilde{S}ₗₗ | Integrated signal of one cell in detector |
| | grid low, object grid low, object present |

| | |
|---|---|
| ${\tilde{V}}_{oh} = \frac{{\tilde{S}}_{hh} - {\tilde{S}}_{hl}}{{\tilde{S}}_{hh} + {\tilde{S}}_{hl}}$ | Object grid visibility in the high signal region of the detector grid fringe in the object’s shadow. |
| ${\tilde{V}}_{ol} = \frac{{\tilde{S}}_{lh} - {\tilde{S}}_{ll}}{{\tilde{S}}_{lh} + {\tilde{S}}_{ll}}$ | Object grid visibility in the low signal region of the detector grid fringe in the object’s shadow. |
| ${\tilde{V}}_{dh} = \frac{{\tilde{S}}_{hh} - {\tilde{S}}_{lh}}{{\tilde{S}}_{hh} + {\tilde{S}}_{lh}}$ | Detector grid visibility in the high signal region of the object grid fringe in the object’s shadow. |
| ${\tilde{V}}_{dl} = \frac{{\tilde{S}}_{hl} - {\tilde{S}}_{ll}}{{\tilde{S}}_{hl} + {\tilde{S}}_{ll}}$ | Detector grid visibility in the low signal region of the object grid fringe in the object’s shadow. |

The response usually can be envisioned as a ratio of the grid visibilities with and without the object present, to quantify how the object changes the visibility:

| | |
|---|---|
| ${ρ}_{oh} = \frac{{\tilde{V}}_{oh}}{{V}_{oh}}$ | Object grid visibility ratio with and without object present in high region of detector grid fringe. |
| ${ρ}_{ol} = \frac{{\tilde{V}}_{ol}}{{V}_{ol}}$ | Object grid visibility ratio with and without object present in low region of detector grid fringe. |
| ${ρ}_{dh} = \frac{{\tilde{V}}_{dh}}{{V}_{dh}}$ | Detector grid visibility ratio with and without object present in high region of object grid fringe. |
| ${ρ}_{dl} = \frac{{\tilde{V}}_{dl}}{{V}_{dl}}$ | Detector grid visibility ratio with and without object present in low region of object grid fringe. |

Then, to correct for spectral changes due to wavelength-dependent absorption by the object altering the transmissivity of the grids (also referred to as beam hardening), the object grid visibility ratio is divided by the detector grid visibility ratio:

| | |
|---|---|
| ${R}_{hh} = \frac{{ρ}_{oh}}{{ρ}_{dh}}$ | Beam hardened corrected visibility ratio, object grid fringe high, detector grid fringe high region. |
| ${R}_{lh} = \frac{{ρ}_{oh}}{{ρ}_{dl}}$ | Beam hardened corrected visibility ratio, object grid fringe high, detector grid fringe low region. |
| ${R}_{hl} = \frac{{ρ}_{ol}}{{ρ}_{dh}}$ | Beam hardened corrected visibility ratio, object grid fringe low, detector grid fringe high region. |
| ${R}_{ll} = \frac{{ρ}_{ol}}{{ρ}_{dl}}$ | Beam hardened corrected visibility ratio, object grid fringe low, detector grid fringe low region. |

Thus, a necessary condition for, e.g., Rₕₕ=1 and Rₗₕ=1 (as one might expect for a non-scattering object), is that ρ_{dh}−ρ_{dl}. Requiring R=1 for all combinations of fringe locations implies that all ρ must be equal. Conversely, all ρ being equal is sufficient to ensure that R=1.

$${ρ}_{oh} = \frac{{\tilde{S}}_{hh} - {\tilde{S}}_{hl}}{{\tilde{S}}_{hh} + {\tilde{S}}_{hl}} \frac{{S}_{hh} + {S}_{hl}}{{S}_{hh} - {S}_{hl}}$$

$${ρ}_{ol} = \frac{{\tilde{S}}_{lh} - {\tilde{S}}_{ll}}{{\tilde{S}}_{lh} + {\tilde{S}}_{ll}} \frac{{S}_{lh} + {S}_{ll}}{{S}_{lh} - {S}_{ll}}$$

$${ρ}_{dh} = \frac{{\tilde{S}}_{hh} - {\tilde{S}}_{lh}}{{\tilde{S}}_{hh} + {\tilde{S}}_{lh}} \frac{{S}_{hh} + {S}_{lh}}{{S}_{hh} - {S}_{lh}}$$

$${ρ}_{dl} = \frac{{\tilde{S}}_{hl} - {\tilde{S}}_{ll}}{{\tilde{S}}_{hl} + {\tilde{S}}_{ll}} \frac{{S}_{hl} + {S}_{ll}}{{S}_{hl} - {S}_{ll}}$$

Therefore, a uniform reduction in visibility signal due to the presence of the object will result in all ρ=1, and thus result in no artifacts.

It can be immediately seen that in regions where the object casts no shadow, R remains unity; with \tilde{V}=V, ρ=1 for both detector and object grating, and thus R=1. So, if there are spectral effects causing artifacts, they will not be present in regions of no attenuation. Further, the spectral effects can be observed to increase with increasing attenuation, making such artifacts more prominent in the high attenuation regions.

The conditions necessary for ρ to remain equal as the beam is hardened can be established by taking the total derivative with respect to the \tilde{S}'s:

$${δρ}_{oh} = \frac{{S}_{hh} + {S}_{hl}}{{S}_{hh} - {S}_{hl}} [ ( \frac{1}{{\tilde{S}}_{hh} + {\tilde{S}}_{hl}} - \frac{{\tilde{S}}_{hh} - {\tilde{S}}_{hl}}{{( {\tilde{S}}_{hh} + {\tilde{S}}_{hl} )}^{2}} ) δ {\tilde{S}}_{hh} - ( \frac{1}{{\tilde{S}}_{hl} + {\tilde{S}}_{ll}} + \frac{{\tilde{S}}_{hl} - {\tilde{S}}_{ll}}{{( {\tilde{S}}_{hh} + {\tilde{S}}_{hl} )}^{2}} ) δ {\tilde{S}}_{hl} ]$$

$${δρ}_{ol} = \frac{{S}_{lh} + {S}_{ll}}{{S}_{lh} - {S}_{ll}} [ ( \frac{1}{{\tilde{S}}_{lh} + {\tilde{S}}_{ll}} - \frac{{\tilde{S}}_{lh} - {\tilde{S}}_{ll}}{{( {\tilde{S}}_{lh} + {\tilde{S}}_{ll} )}^{2}} ) δ {\tilde{S}}_{lh} - ( \frac{1}{{\tilde{S}}_{lh} + {\tilde{S}}_{ll}} + \frac{{\tilde{S}}_{lh} - {\tilde{S}}_{ll}}{{( {\tilde{S}}_{lh} + {\tilde{S}}_{ll} )}^{2}} ) δ {\tilde{S}}_{ll} ]$$

$${δρ}_{dh} = \frac{{S}_{hh} + {S}_{lh}}{{S}_{hh} - {S}_{lh}} [ ( \frac{1}{{\tilde{S}}_{hh} + {\tilde{S}}_{lh}} - \frac{{\tilde{S}}_{hh} - {\tilde{S}}_{lh}}{{( {\tilde{S}}_{hh} + {\tilde{S}}_{lh} )}^{2}} ) δ {\tilde{S}}_{hh} - ( \frac{1}{{\tilde{S}}_{hh} + {\tilde{S}}_{lh}} + \frac{{\tilde{S}}_{hh} - {\tilde{S}}_{lh}}{{( {\tilde{S}}_{hh} + {\tilde{S}}_{lh} )}^{2}} ) δ {\tilde{S}}_{lh} ]$$

$${δρ}_{dl} = \frac{{S}_{hl} + {S}_{ll}}{{S}_{hl} - {S}_{ll}} [ ( \frac{1}{{\tilde{S}}_{hl} + {\tilde{S}}_{ll}} - \frac{{\tilde{S}}_{hl} - {\tilde{S}}_{ll}}{{( {\tilde{S}}_{hl} + {\tilde{S}}_{ll} )}^{2}} ) δ {\tilde{S}}_{hl} - ( \frac{1}{{\tilde{S}}_{hl} + {\tilde{S}}_{ll}} + \frac{{\tilde{S}}_{hl} - {\tilde{S}}_{ll}}{{( {\tilde{S}}_{hl} + {\tilde{S}}_{ll} )}^{2}} ) δ {\tilde{S}}_{ll} ]$$

If we start from the no-object condition, then S=\tilde{S}∀S and:

$${ρ}_{oh} = ( \frac{1}{{S}_{hh} - {S}_{hl}} - \frac{1}{{S}_{hh} + {S}_{hl}} ) δ {\tilde{S}}_{hh} - ( \frac{1}{{S}_{hh} - {S}_{hl}} + \frac{1}{{S}_{hh} + {S}_{hl}} ) δ {\tilde{S}}_{hl}$$

$${δρ}_{ol} = ( \frac{1}{{S}_{lh} - {S}_{ll}} - \frac{1}{{S}_{lh} + {S}_{ll}} ) δ {\tilde{S}}_{lh} - ( \frac{1}{{S}_{lh} - {S}_{ll}} + \frac{1}{{S}_{lh} + {S}_{ll}} ) δ {\tilde{S}}_{ll}$$

$${δρ}_{dh} = ( \frac{1}{{S}_{hh} - {S}_{lh}} - \frac{1}{{S}_{hh} + {S}_{lh}} ) δ {\tilde{S}}_{hh} - ( \frac{1}{{S}_{hh} - {S}_{lh}} + \frac{1}{{S}_{hh} + {S}_{lh}} ) δ {\tilde{S}}_{lh}$$

$${δρ}_{dl} = ( \frac{1}{{S}_{hl} - {S}_{ll}} - \frac{1}{{S}_{hl} + {S}_{ll}} ) δ {\tilde{S}}_{hl} - ( \frac{1}{{S}_{hl} - {S}_{ll}} + \frac{1}{{S}_{hl} + {S}_{ll}} ) δ {\tilde{S}}_{ll}$$

$$or :$$

$${δρ}_{oh} = \frac{2 {S}_{hl} δ {\tilde{S}}_{hh} - 2 {S}_{hh} δ {\tilde{S}}_{hl}}{{S}_{hh}^{2} - {S}_{hl}^{2}}$$

$${δρ}_{ol} = \frac{2 {S}_{ll} δ {\tilde{S}}_{lh} - 2 {S}_{lh} δ {\tilde{S}}_{ll}}{{S}_{lh}^{2} - {S}_{ll}^{2}}$$

$${δρ}_{dh} = \frac{2 {S}_{lh} δ {\tilde{S}}_{hh} - 2 {S}_{hh} δ {\tilde{S}}_{lh}}{{S}_{hh}^{2} - {S}_{lh}^{2}}$$

$${δρ}_{dl} = \frac{2 {S}_{ll} δ {\tilde{S}}_{hl} - 2 {S}_{hl} δ {\tilde{S}}_{ll}}{{S}_{hl}^{2} - {S}_{ll}^{2}}$$

which can be written as a matrix equation:

$$\vec{δρ} = \overleftrightarrow{A} \vec{δ \tilde{S}} .$$

The condition that all ρ remain the same means \vec{δp}=δp\vec{1}. Thus, the necessary condition of the lack of artifacts can be solved for:

$$\vec{δ \tilde{S}} = δρ {\overleftrightarrow{A}}^{- 1} \vec{1} .$$

For the special case of ρ remaining unchanged, [symbol] must be singular and \vec{δ\tilde{S}} within its null space. At first it appears unpromising for avoiding spectral artifacts, as it requires a highly cooperative target material that attenuates the spectrum in a certain way. Also, the signals are expected to be attenuated rather than amplified, so that every δ\tilde{S}<0.

Special cases can exist for identical object and detector gratings. For example, while it might not always be possible to choose the object being imaged, the gratings and associated characteristics can be chosen. In many examples, the thickness, composition, and projected periods of the object and detector grids can be matched as closely as possible. Where this is achieved, Sₗₕ=Sₕₗ and \tilde{S}ₗₕ−\tilde{S}ₕₗ. Consequently, Vₒₕ=V_{dh} ≡Vₕ, Vₒₗ=V_{dl} ≡Vₗ, \tilde{V}ₒₕ=\tilde{V}_{dh} ≡\tilde{V}ₕ, and \tilde{V}ₒₗ=\tilde{V}_{dl}≡\tilde{V}ₗ. This leads to ρₒₕ=ρ_{dh} and pₒₗ=ρ_{dl}. Thus Rₕₕ=1, Rₗₕ=1/Rₕₗ, and Rₗₗ=1. It is now the mixed high/low fringe regions between the two gratings that give rise to the beam hardening fringe artifacts, while the high/high and low/low regions correct out as desired.

Special cases can also exist for blurred object gratings. The finite size of the x-ray spot and imperfect source grating corrections will, in practice, lead to lower visibility of the object grating compared to the detector grating. To estimate the effect of grid blurring, it can be assumed that for perfect resolution the detector grating and object grating are identical except for a reflection and 90-degree rotation, but due to blurring a fraction f of the object grating shadow signal spills over into the unshaded region and vice versa. The subscript u is used to signify the unblurred quantity, so for example S_{hh,u}, S_{hl,u}, S_{ll,u}, and S_{ll,u} are the unblurred signals. If identical unblurred shadows of object and detector grid (S_{hl,u}=S_{lh,u}) are assumed:

$${S}_{hh} = ( 1 - f ) {S}_{hh , u} + {fS}_{hl , u}$$

$${S}_{hl} = ( 1 - f ) {S}_{hl , u} + {fS}_{hh , u}$$

$${S}_{lh} = ( 1 - f ) {S}_{hl , u} + {fS}_{ll , u}$$

$${S}_{ll} = ( 1 - f ) {S}_{ll , u} + {fS}_{hl , u}$$

The blurring in this case is geometrical, and thus spectrum independent. Therefore, the shadowed signals have the same relationship to the corresponding unblurred signals as do the unshaded signals.

$${V}_{oh} = \frac{( 1 - f ) {S}_{hh , u} + {fS}_{hl , u} - ( 1 - f ) {S}_{hl \cdot u} - {fS}_{hh , u}}{( 1 - f ) {S}_{hh , u} + {fS}_{hl , u} + ( 1 - f ) {S}_{hl , u} + {fS}_{hh , u}} = \frac{( 1 - 2 f ) {S}_{hh , u} - ( 1 - 2 f ) {S}_{hl , u}}{{S}_{hh , u} + {S}_{hl , u}} = ( 1 - 2 f ) {V}_{oh , u} .$$

$${V}_{dh} = \frac{( 1 - f ) {S}_{hh , u} + {fS}_{hl , u} - ( 1 - f ) {S}_{hl , u} - {fS}_{ll , u}}{( 1 - f ) {S}_{hh , u} + {fS}_{hl , u} + ( 1 - f ) {S}_{hl , u} + {fS}_{ll , u}} = \frac{( 1 - f ) {S}_{hh , u} - ( 1 - 2 f ) {S}_{hl , u} - {fS}_{ll , u}}{( 1 - f ) {S}_{hh , u} + {S}_{hl , u} + {fS}_{ll , u}}$$

### Similarly:

$${V}_{ol} = ( 1 - 2 f ) {V}_{ol , u}$$

$${V}_{dl} = \frac{{fS}_{hh , u} + ( 1 - 2 f ) {S}_{hl , u} - ( 1 - f ) {S}_{ll , u}}{{fS}_{hh , u} + {S}_{hl , u} + ( 1 - f ) {S}_{ll , u}}$$

Because (1-2f) factors out of the object grating visibilities, it cancels when taking the ρ ratios for the object grating. Consequently, ρₒₕ=ρ_{oh,u} and ρₒₗ=ρ_{ol,u}. The same does not apply for the detector grating p ratios. This breaks the symmetry that was had in the perfect resolution case, so the assurance that Rₕₕ=Rₗₗ=1 is lacking.

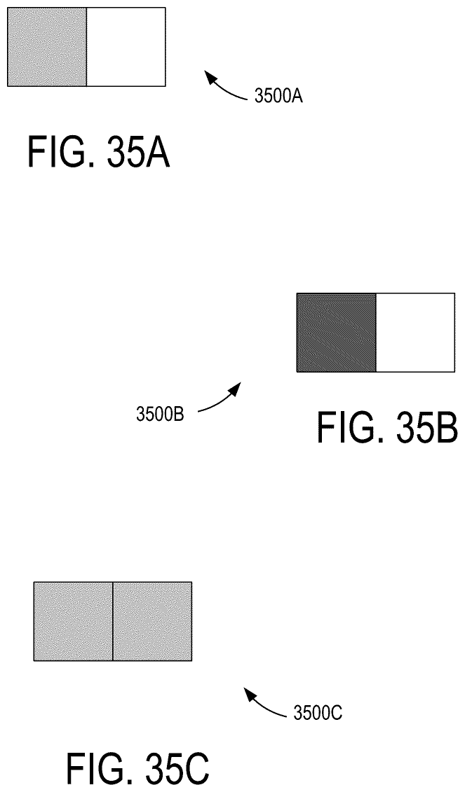

For many sequential moiré imaging examples, only a two-square cell for tiling need be considered. The detector, object, and analyzer grating all have a tiling cell like cell pattern 3500A shown in FIG. 35A. The maximum transmission regions of the moiré fringes can look similar to the cell pattern 3500B in FIG. 35B while the minimum transmission regions can look similar to the cell pattern 3500C in FIG. 35C.

Integrated signals can be defined:

| | |
|---|---|
| Sₒₕ | Integrated signal of one cell in the object grid |
| | image in a high signal part of the fringe. |
| Sₒₗ | Integrated signal of one cell in the object grid |
| | image in a low signal part of the fringe. |
| S_{dh} | Integrated signal of one cell in the detector grid |
| | image in a high signal part of the fringe. |
| S_{dl} | Integrated signal of one cell in the detector grid |
| | image in a low signal part of the fringe. |

The visibilities are:

| | |
|---|---|
| ${V}_{o} = \frac{{S}_{oh} - {S}_{ol}}{{S}_{oh} + {S}_{ol}}$ | Object grid visibility. |
| ${V}_{d} = \frac{{S}_{dh} - {S}_{dl}}{{S}_{dh} + {S}_{dl}}$ | Detector grid visibility. |

And for the attenuated beam:

| | |
|---|---|
| \tilde{S}ₒₕ | Signal of a cell in the object grid image in a |
| | high part of the fringe with object present. |
| \tilde{S}ₒₗ | Signal of a cell in the object grid image in a |
| | low part of the fringe with object present. |
| \tilde{S}_{dh} | Signal of a cell in the detector grid image in a |
| | high part of the fringe with object present. |
| \tilde{S}_{dl} | Signal of a cell in the detector grid image in a |
| | low part of the fringe with object present. |

| | |
|---|---|
| ${\tilde{V}}_{o} = \frac{{\tilde{S}}_{oh} - {\tilde{S}}_{ol}}{{\tilde{S}}_{oh} + {\tilde{S}}_{ol}}$ | Object grid visibility with object present. |
| ${\tilde{V}}_{d} = \frac{{\tilde{S}}_{dh} - {\tilde{S}}_{dl}}{{\tilde{S}}_{dh} + {\tilde{S}}_{dl}}$ | Detector grid visibility with object present. |

Again, the response can be envisioned as ratios of the grating visibilities with and without the object present, to quantify how the object changes the visibility.

| | |
|---|---|
| ${ρ}_{o} = \frac{{\tilde{V}}_{o}}{{V}_{o}}$ | Object grid visibility ratio with and without object present. |
| ${ρ}_{d} = \frac{{\tilde{V}}_{d}}{{V}_{d}}$ | Detector grid visibility ratio with and without object present. |

This leads to a single beam hardened corrected visibility ratio:

| | |
|---|---|
| $R = \frac{{ρ}_{o}}{{ρ}_{d}}$ | Beam hardened corrected visibility ratio. |

The same fringe-dependent beam hardening artifacts seen with the cross grid moiré set-up are not seen with only one ratio. In many examples, the detector grating and object grating are provided with the same thickness and material composition. In this case, for an ideal point source, the detector grid attenuation will be the same as that of the object grid so that Sₒₕ=S_{dh}, Sₒₗ=S_{dl}, \tilde{S}ₒₕ=\tilde{S}_{dh}, and \tilde{S}ₒₗ=\tilde{S}_{dl}. For this case, Vₒ=V_{d} and \tilde{V}ₒ=\tilde{V}_{d} so that ρₒ=ρ_{d} and, in the absence of scattering, R=1. In practice, it can be seen that the object grating visibility is usually significantly less than that for the detector grating, which may be due to the blurring from a finite sized x-ray source and imperfect corrections with the source grating.

To estimate the effect of grid blurring, a fraction f of the object grating shadow signal can be assumed to spill over into the unshaded region and vice versa. For the unblurred signal as above, the blurred object grid signal is:

$${S}_{oh} = ( 1 - f ) {S}_{dh} + {fS}_{dl}$$

$${S}_{ol} = ( 1 - f ) {S}_{dl} + {fS}_{dh}$$

$${V}_{o} = \frac{( 1 - f ) {S}_{dh} + {fS}_{dl} - ( 1 - f ) {S}_{dl} - {fS}_{dh}}{( 1 - f ) {S}_{dh} + {fS}_{dl} + ( 1 - f ) {S}_{dl} + {fS}_{dh}} = \frac{( 1 - 2 f ) {S}_{dh} - ( 1 - 2 f ) {S}_{dl}}{{S}_{dh} + {S}_{dl}} = ( 1 - 2 f ) {V}_{d} .$$

The blurring in this case is geometrical, and thus spectrum independent. Therefore:

$${\tilde{V}}_{o} = ( 1 - 2 f ) {\tilde{V}}_{d} .$$

Under this assumption, ρₒ is unchanged and, again R corrects to 1.

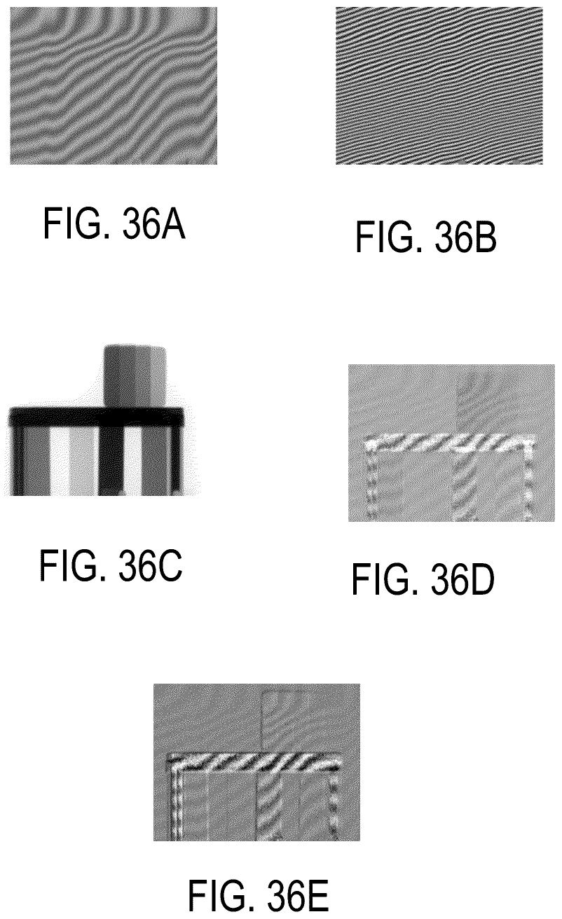

However, as discussed hereinabove, it has been found that the fringe artifacts that can occur in cross-moiré scanning can be reduced. As discussed previously, in cross-moiré scanning, a rectilinearly periodic perforated analyzer grating can be used to form moiré fringes together with both an object grating and a detector grating. By stepping the analyzer grating (or the detector and object gratings, or both) across one or more periods in both x and y Cartesian directions, the phase, contrast, and absorption can be measured for both detector and object gratings. However, when processing these data sets, artifacts appear in the final images that tend to follow the paths of the moiré fringes for the object grating or superimposed fringes of object and detector ratings. These artifacts can be seen more prominently in high attenuation regions. Examples of such artifacts obtained from cross-grid scanning can be seen in FIGS. 36A-36E. For example, FIG. 36A shows object grating moiré fringes, FIG. 36B shows detector grating moiré fringes, and FIGS. 36C-36E show absorption, visibility ratio, and phase images, respectively.

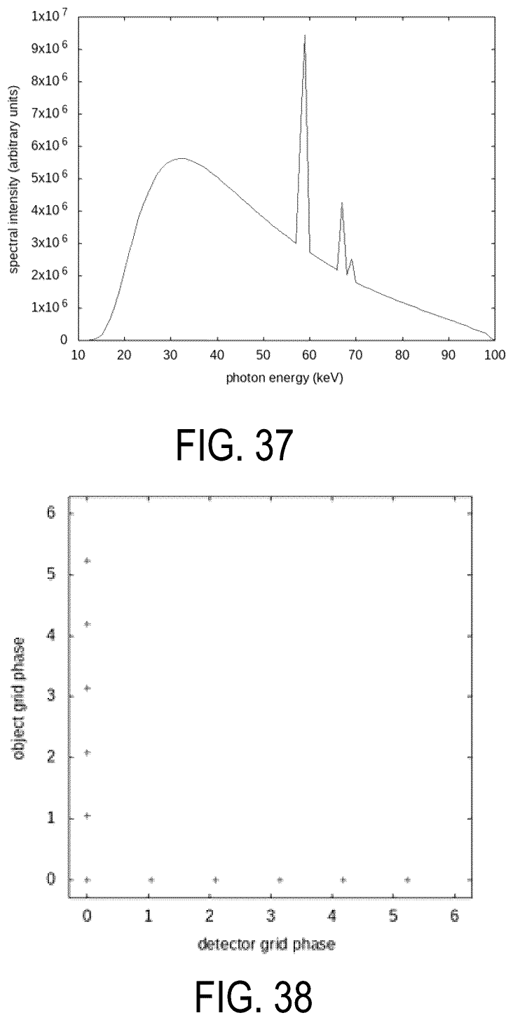

Cross grid fringe artifacts were investigated through simulation. A spectral calculator (SpekCalc) was used to generate a spectrum for an x-ray source based on a 100 kV end point energy, which is a typical x-ray source spectrum filtered by 2 mm Al. Such a spectrum is shown in FIG. 37. A grating thickness was specified for the object, detector, and analyzer gratings. The material used for the gratings was assumed to be lead. An aluminum slab with thickness chosen to reduce the x-ray intensity by a factor of e⁻² (two effective mean free paths for this spectrum) was chosen for the object, and positioned to occupy a right half of the detector. The attenuation coefficients for lead and aluminum were taken from the NIST (National Institute of Standards and Technology) x-ray attenuation database. The spectrum was propagated through every combination of gratings and object, and the integrated resulting spectrum was used for the detector signal behind that set of obstructing devices. For every pixel, an average was taken over the signals weighted by the fraction of the geometry occupied by the combination of obstructing devices in that pixel. This gives an x-ray image for the particular geometry chosen. The analyzer grating was then phase stepped with respect to the detector and object gratings (e.g., translated perpendicularly with respect to the beam path at selected increments) in order to obtain a sequence of phase step images that were processed using available software for extracting absorption, visibility ratio, and phase images from moiré scanning measurements. For the base simulation, the phase with respect to the object grating was held fixed at zero while the detector grating phase was stepped by π/3 for six phase steps; then the detector grating phase was held at zero while the object grating phase was stepped by π/3 for six phase steps. FIG. 38 shows a 2-D plot of the phase combinations used for the base phase scanning simulation. These phase steps can correspond to phase steps that are taken in current 2-D moiré scanning measurements.

Analysis methods for collected scanned moiré measurements are now discussed. Consider the intensity I(x, y; μ, v) at pixel (x, y), with horizontal phase p and the vertical phase v of the analyzer grating with respect to the detector and object gratings, respectively. At this pixel, the x-rays must pass through a thickness of material T(x, y; μ, v). If r is the attenuation length for x-rays in the material, then:

$$I ( x , y ; μ , v ) = {I}_{0} exp [ - \frac{T ( x , y ; μ , v )}{τ} ] .$$

T(x, y; μ, v) is the sum of the thicknesses of the object grating Tₒ(x, y; v) and detector grating T_{d}(x, y; μ). As the lowest order terms (zeroth and first) are of interest in the Fourier transform with respect to the phase, each of these can be decomposed into a mean value for the grid T̅(x, y) and an oscillatory term δTₒ(x, y) cos[v+ϕₒ(x, y)] or δT_{d}(x, y) cos[μ+ϕ_{d}(x, y)]:

$$I ( x , y ; μ , v ) = {I}_{0} exp [ - \frac{1}{τ} ( {\overline{T}}_{d} ( x , y ) + δ {T}_{d} ( x , y ) cos [ μ + {ϕ}_{d} ( x , y ) ] + {\overline{T}}_{o} ( x , y ) + δ {T}_{o} ( x , y ) cos [ v + {ϕ}_{o} ( x , y ) ] ) ]$$

The x-rays traverse a mean free path of:

$$α ( x , y ; μ , v ) = - ln [ I ( x , y ; μ , v ) ] = ln [ {I}_{0} ] - \frac{1}{τ} ( {\overline{T}}_{d} ( x , y ) + δ {T}_{d} ( x , y ) cos [ μ + {ϕ}_{d} ( x , y ) ] + {\overline{T}}_{o} ( x , y ) + δ {T}_{o} ( x , y ) cos [ v + {ϕ}_{o} ( x , y ) ] ) = \overline{α} ( x , y ) + {V}_{d} ( x , y ) cos [ μ + {ϕ}_{d} ( x , y ) ] + {V}_{o} ( x , y ) cos [ v + {ϕ}_{o} ( x , y ) ]$$

The Fourier decomposition can then be performed on α(x, y; μ, v). For the detector grating this returns the phase ϕ_{d}(x, y), amplitude V_{d}(x, y), and mean M_{d}(x, y)=α̅(x, y)+δαₒ(x, y) cos[<v>+ϕₒ(x, y)]. For the object grating, the phase ϕₒ(x, y), amplitude Vₒ(x, y), and mean of Mₒ(x, y)=α̅(x, y)+V_{d}(x, y) cos[<μ>+ϕ_{d}(x, y)] can be obtained. This provides sufficient information to solve directly for α̅(x, y). In fact, two separate solutions for α̅(x, y) are:

$${\overline{α}}_{d} ( x , y ) = ( {M}_{d} ( x , y ) - {V}_{o} ( x , y ) cos [ 〈 v 〉 + {ϕ}_{o} ( x , y ) ] )$$

$$and$$

$${\overline{α}}_{o} ( x , y ) = ( {M}_{o} ( x , y ) - {V}_{d} ( x , y ) cos [ 〈 μ 〉 + {ϕ}_{d} ( x , y ) ] ) .$$

Thus, α̅(x, y) can be estimated as the average of these two:

$$\overline{α} ( x , y ) = \frac{1}{2} [ ( {M}_{d} ( x , y ) - {V}_{o} ( x , y ) cos [ 〈 v 〉 + {ϕ}_{o} ( x , y ) ] ) + ( {M}_{o} ( x , y ) - {V}_{d} ( x , y ) cos [ 〈 μ 〉 + {ϕ}_{d} ( x , y ) ] ) ]$$

Now, with:

$$I ( x , y ; μ , v ) = exp [ - α ( x , y ; μ , v ) ] = exp [ - \overline{α} ( x , y ) ] exp [ - {V}_{d} ( x , y ) cos [ μ + {ϕ}_{d} ( x , y ) ] ] exp [ - {V}_{o} ( x , y ) cos [ v + {ϕ}_{o} ( x , y ) ] ]$$

and assuming the Vs are small:

$$exp [ - {V}_{d} ( x , y ) cos [ μ + {ϕ}_{d} ( x , y ) ] ] \approx 1 - {V}_{d} ( x , y ) cos [ μ + {ϕ}_{d} ( x , y ) ]$$

$$exp [ - {δα}_{o} ( x , y ) cos [ v + {ϕ}_{o} ( x , y ) ] ] \approx 1 - {V}_{o} ( x , y ) cos [ v + {ϕ}_{o} ( x , y ) ]$$

the first order in δα can be found:

$$I ( x , y ; μ , v ) \approx exp [ - \overline{α} ( x , y ) ] ( 1 - {V}_{d} ( x , y ) cos [ μ + {ϕ}_{d} ( x , y ) ] - {V}_{o} ( x , y ) cos [ v + {ϕ}_{o} ( x , y ) ] ) .$$

In other words, the Vs are the grid visibilities, and the phase shift function of the grid image will have an additional π to cancel out that minus sign. This π is removed when subtracting the flat field phase shift, and is unobservable.

There are several methods that can be used to correct out certain effects that may have occurred during data collection. A region of the phase step images that ideally does not have the analyzer grating in it can be integrated to obtain the relative exposure of each phase step, the phase steps can then all be normalized or otherwise corrected for variations in exposure.

In experimental arrangements, the actual phase of the analyzer grating with respect to the detector and object gratings can be measured to within approximately 0.01 radians (depending on noise, stage accuracy, and number of steps) by Fourier transforming the phase step images, selecting the pixel corresponding to the analyzer grid first harmonic—or, alternately, to the corresponding moiré fringe. The phase of this pixel gives the phase of the analyzer grating in that image. For example, if the translation stage has worse accuracy than about 0.0016 of a period (corresponding to 0.01 radians of phase), this can help to determine the actual phase value. However, it will be appreciated that other measurement accuracies are possible in various systems and arrangements.

To correct for differences between the measured phase and assumed phase, for each pixel (x, y) the values α̅(x, y), V_{d}(x, y), Vₒ(x, y), ϕ_{d}(x, y), and ϕₒ(x, y) can be found using the assumed phase of two step sequences, where each sequence has one grid with which it remains at zero phase and is stepped in N equal intervals with respect to the other grid, giving the analysis from the beginning of this section. This produces 2N data points Iₘ(x, y; μ, v) at known (μ, v). The correction for the known (μ, v) can be performed by minimizing:

$${χ}^{2} = {\sum }_{m = 1}^{2 N} {( {I}_{m} ( x , y ; μ , v ) - I ( x , y ; μ , v ) )}^{2} .$$

by varying α̅(x, y), V_{d}(x, y), Vₒ(x, y), ϕ_{d}(x, y), and ϕₒ(x, y) with a Levenberg-Marquardt algorithm.

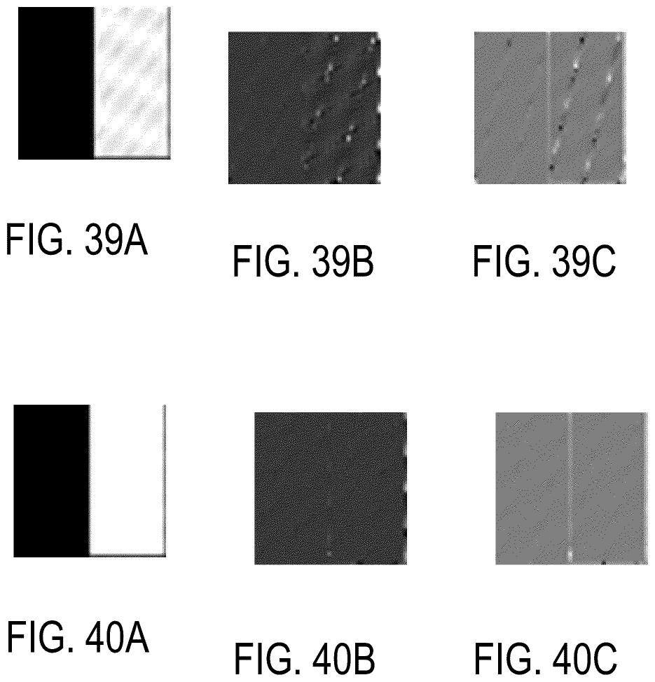

A standard analysis was performed on the simulated data and the results are shown in FIGS. 39A-39C, which correspond to images of absorption coefficient, visibility ratio, and phase shift, respectively. Clear moiré fringe artifacts can be seen, indicating that the simulation method captures a physics that can reproduce these artifacts. The simulation was repeated with a monochromatic 50 keV spectrum and the corresponding results are shown in FIG. 40A-40C. The results correspond to images of absorption coefficient, visibility ratio, and phase shift, respectively. The fringe artifacts are significantly reduced with a monochromatic spectrum, indicating that the main source of the artifacts is spectral. The presence of less prominent fringe artifacts remaining in the monochromatic simulation results suggests there are also other, non-spectral contributions of lesser importance.

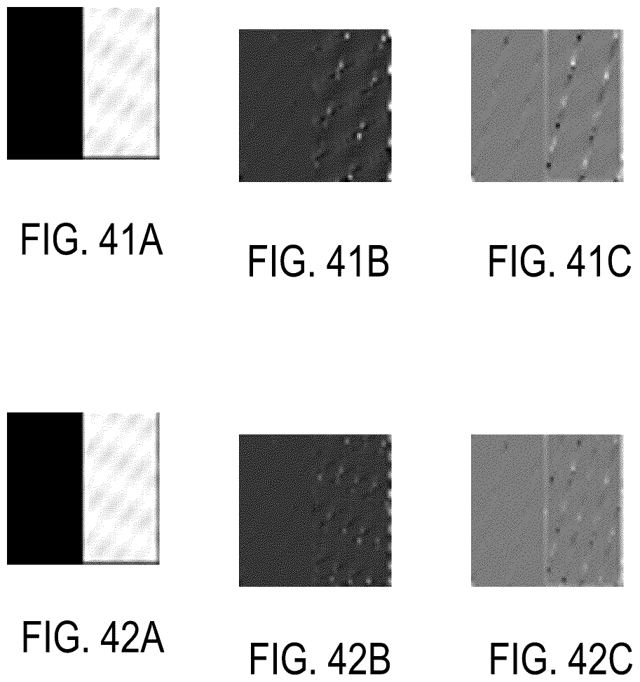

Various techniques can be employed to reduce artifacts due to spectral averaging. If a set of two orthogonal phase scans produces artifacts corresponding to the moiré fringes, then it can follow that shifting the phase of the static grid during the scan by π radians will produce artifacts with the opposite sense of the fringe artifacts—e.g., regions that were once darker become brighter, and vice versa. Taking the average of the standard and π-shifted images can help to average out the artifacts. FIGS. 41A-42C illustrate the effect of an averaging of the zero phase shift analysis with the π-shifted analysis. More specifically, FIGS. 41A-41C show the uncorrected base analysis of images of absorption coefficient, visibility ratio, and phase shift, respectively. FIGS. 42A-42C show the corrected base analysis of images of absorption coefficient, visibility ratio, and phase shift, respectively. Thus, as can be seen by comparing the uncorrected and corrected images, in practice, the artifacts are reduced but the improvement is often not complete or not necessarily substantial.

Another way to correct the artifacts due to spectral averaging is to acquire additional data in additional parts of the phase plane to fill in information missing from the two phase step measurement sets with fixed zero phase of the stationary grid that are shown in FIG. 38. These additional measurements are included directly as data into the χ² minimization step in addition to the original sets of orthogonal steps used for the initial parameter estimates. In many examples, the additional measurements are not used for the initial parameter estimates and instead only for the final minimization step. As such, they can have arbitrary phase coordinates within the [0:2π] range.

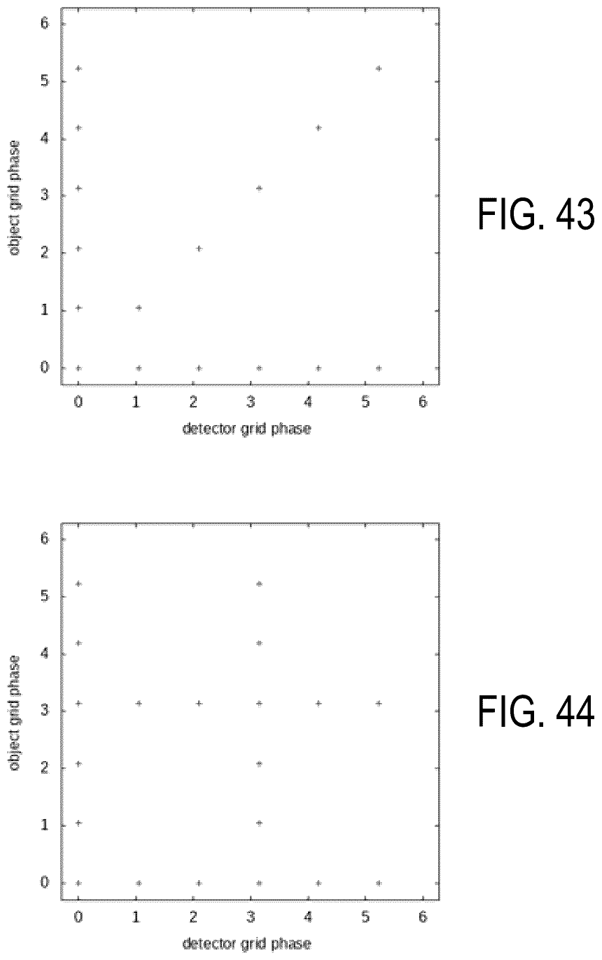

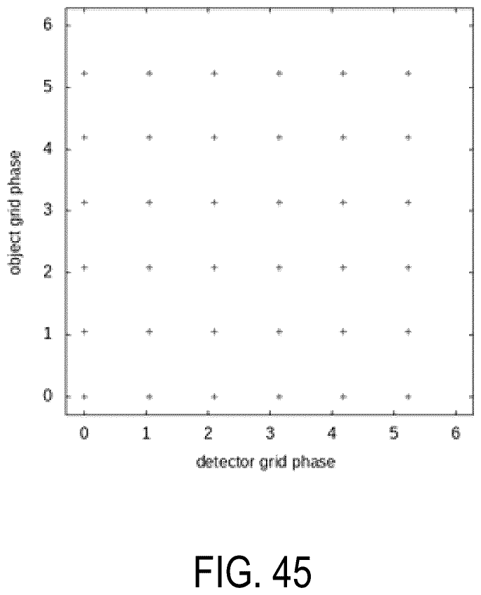

Three sequences of phase step measurements that provided additional phase information were selected and examined, though it will be appreciated that other sequences may be used. FIG. 43 shows an example of a “diagonal” phase scanning sequence, i.e., in addition to the original orthogonal sets (e.g., forming an “L” shape) an additional set of measurements is taken where the analyzer grid has the same phase with respect to both detector and object gratings. FIG. 44 shows an example of a π-shift phase scanning sequence in which two additional sets are taken with the analyzer grid set to π radians of phase relative to one stationary grid while stepping relative to the other, and vice versa, which can efficiently average over spectral effects. FIG. 45 shows an example of a lattice phase scanning sequence, where a phase measurement is taken for every combination m, n∈{0,1,2,3,4,5} with phase (2πm/6, 2πn/6).

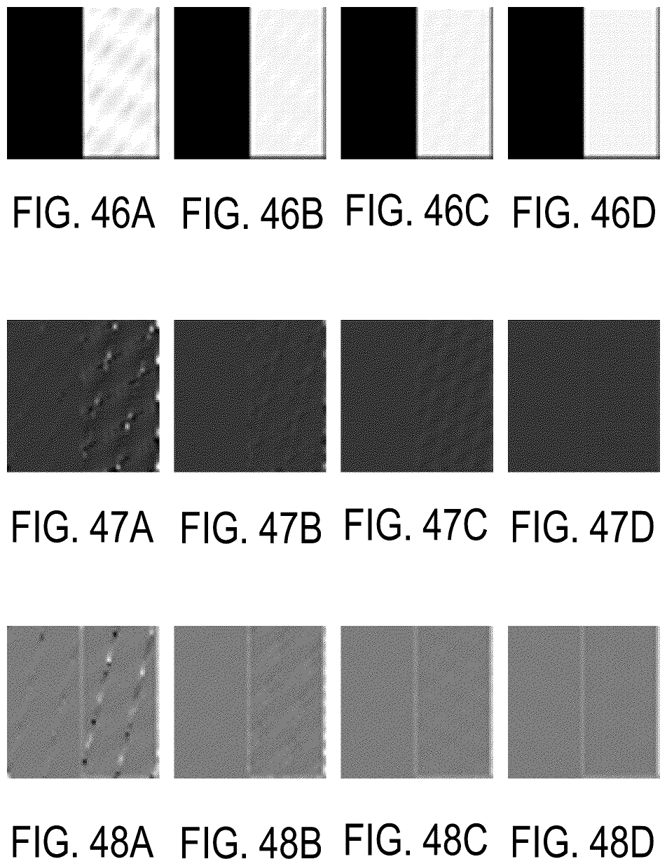

Image results from adding the additional phase step sets into the final fit are shown in FIG. 46A-48D. In particular, FIGS. 46A-46D show images for the absorption coefficient for base phase steps, base+diagonal phase steps, base+pi-shift phase steps, and a latticed sequence, respectively. FIGS. 47A-47D show images for the visibility for base phase steps, base+diagonal phase steps, base+pi-shift phase steps, and a latticed sequence, respectively. FIGS. 48A-48D show images for the phase shift for base phase steps, base+diagonal phase steps, base+pi-shift phase steps, and a latticed sequence, respectively. It can be seen from the image results that adding additional phase step sets can significantly reduce the artifacts. Also, the magnitude of the residual artifacts reduces with increasing numbers of data sets to fill in the unsampled region of the phase plane.

### Overview of Computed Tomography Examples

As mentioned above, applications can include computed tomography (CT). Security screening can rely on x-ray computed tomography (CT) to characterize contents of bags and packages in 3-D, and with the use of dual energy, CT techniques can be configured to perform automatic threat recognition to distinguish explosive and benign materials. However, various problems remain in currently available x-ray CT systems, such as substantial rates of false alarms. Furthermore, the ability to obtain additional material information could improve detection. Gratings-based phase contrast x-ray imaging (GBX) described herein can provide additional signatures in an x-ray image: absorption, similar to conventional imaging; differential phase, proportional to electron density gradients in an object; and dark field, which, when corrected for spectral changes, measures small angle x-ray scattering from object microstructure. Disclosed CT examples can use these signatures can enable improved material discrimination. Some example GBX prototypes described herein can provide a single-view but many examples can perform CT on all three modes.

A lab-scale GBX-CT system was constructed and CT reconstructions of all three signatures were demonstrated. A set of three plastic cylinders of known composition was used to demonstrate quantitative results, indicating the difference between the phase reconstruction's dependence on electron density as compared with the absorption reconstruction's stronger dependence on atomic number. The scatter signal for these materials is zero, consistent with their lack of microstructure. A scatter step wedge, consisting of ZnO particles in epoxy, demonstrates reconstruction of a non-zero scatter signature. These lab-scale measurements provide proof of concept for systems designed for high-energy applications, demonstrate the additional materials signatures provided, and give a baseline understanding of performance for a given system design and total exposure.

Various system geometries may be used, including geometries that are similar to existing CT systems used for security screening. Candidate system geometries can exhibit design tradeoffs between sensitivity to phase and scatter, and ease of construction or operation. In some examples, it can be estimated that a system based on commercial anti-scatter gratings may have a maximum sensitivity of about 0.25 times the system used for lab measurements and will be reduced further by sensitivity gradients across the imaged region. It was found that while the total exposure of some lab measurements may be high, a lower resolution image could be acquired much more quickly.

Suitable operational GBX-CT systems may depart from lab-scale demonstration geometries in various ways in the pursuit of reducing systematic errors, particularly where commercially available components are used. For example, higher sensitivity approaches may use custom gratings and potentially new grating fabrication techniques to make them. Also, mechanical designs may be altered in order to achieve stability to less than 15 km. Alternative approaches to leveraging these signatures may be possible, including approaches geared towards smaller objects (which enable lower energies where cross sections are larger), or leveraging single-view GBX measurements in combination with dual-energy CT, along with developing algorithms that can associate the 2-D signature data with objects identified in the 3-D CT data.

### Acronyms And Abbreviations

CT Computed Tomography
GBX Gratings-based phase contrast x-ray imaging
Z_{eff} Effective atomic number
ρₑ Electron density
ATR Automated threat recognition
PEEK Polyetheretherketone
PVDF Polyvinylidene fluoride
PTFE Polytetrafluoroethylene
ROI Region of interest
CNR Contrast-to-noise ratio

### CT Example Tables

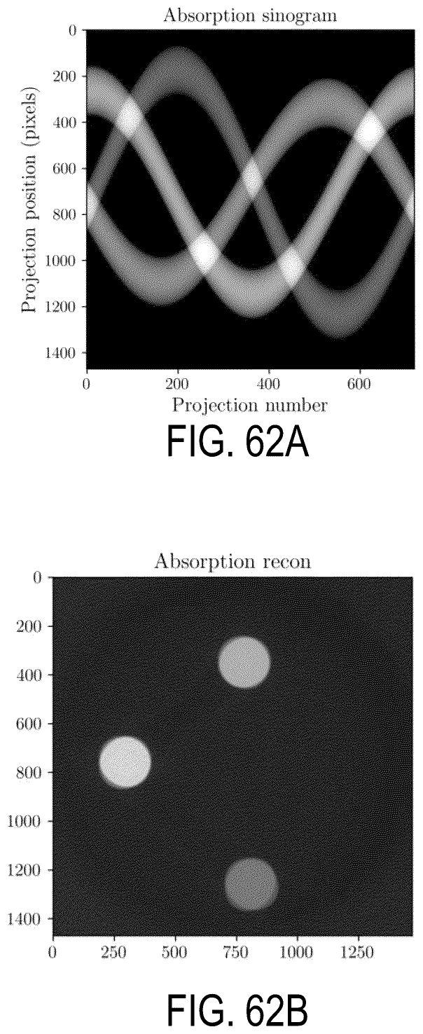

CT Table 1. Contrast-to-noise ratios for absorption, phase, and scatter in the stepwedge.
CT Table 2. Comparison of a weighted average of the known attenuation coefficients, μ, to that extracted from the reconstructed attenuation profiles shown in FIG. 62B.
CT Table 3. A comparison of physical characteristics of the plastics to the reconstructed differential phase values, where the mean and standard deviation of a ROI are reported. The electron density ρe and extracted phase are normalized for direct comparison.
CT Table 4. CNR values for the plastic rods.
CT Table 5. A summary of grid and geometry options explored in this section. A couple of the options show that sensitivity of the baseline system could be reached within a nominal CT system geometry.

### Introduction to CT Examples

Gratings-based phase contrast imaging (GBX) can provide additional material signatures from an x-ray imaging measurement, based on slight deflections of the x-ray beam as it passes through a material in addition to attenuation. In addition to an absorption image, similar to conventional x-ray imaging, the measurement produces a differential phase contrast image which is sensitive to electron density gradients, and a dark field image which is sensitive to material microstructure.

It has been demonstrated that these additional signatures can be detected using spectra similar to those used for checkpoint screening, and that scatter can supplement dual-energy imaging to improve discrimination between explosives and benign materials. Further investigation has shown that this approach can be adapted to a single-view screening system. Disclosed GBX system prototypes were tested to determine their applicability for secondary screening at the checkpoint.

As mentioned above, CT can be an important tool for security screening. CT security screening generally involves the collecting of transmission radiographs at many angles through a sample and performing an image reconstruction to provide a 3-D estimate of object structure. In a radiograph, material parameters can be estimated only approximately, since the pathlength along the beam is unknown. The CT measurement provides the spatial distribution of material in an object, and dual energy CT measurements can be used to estimate the spatial distribution of electron density (ρₑ) and effective atomic number (Z_{eff}) for each voxel of material. This enables the development of Automated Threat Recognition (ATR) systems, which are used to automatically flag potential threat materials. CT systems are now being used for threat detection at checkpoints.

In disclosed examples, CT can be performed based on GBX measurements for each of the three signatures. At a single energy, dark field contrast (pattern visibility loss) accrues exponentially, similar to intensity loss for attenuation measurements, so the dark field projection images can be reconstructed using the same algorithms as the attenuation image. For differential-phase images, if a periodic line patterning element aligned parallel to the rotation axis is used for the measurement, then the differential phase data is along horizontal lines in the projections. This data can be used to reconstruct the phase information by changing the filter that is used in the filtered backprojection reconstruction method. However, as with projection imaging GBX, most prior GBX CT has been done on systems designed for lower energy applications, typically with endpoint energy below 100 keV, and in most cases the impact of spectral effects on the dark field image are not explicitly accounted for. Disclosed examples can include GBX CT systems that have been configured for higher energies with sensitivities suitable for imaging geometries used for security screening.

In analyzing high energy GBX CT measurement feasibility, GBX CT system requirements and tradeoffs between sensitivity, noise, and stability were examined. In some examples, systems can employ typical geometries used for security screening CT, with both direct and indirect imaging. Some example systems can perform GBX CT measurements with a system components configured for high energy, e.g., using an attenuation-based periodic patterning grid, a source grid to enhance pattern visibility, and a detector grid to allow discrimination between spectral and structural contributions to the dark field image modality. The patterns can be imaged directly on a high-resolution detector. Reconstruction of absorption, phase, and scatter signals can be performed. In many examples, systems can produce measurements with suitable contrast-to-noise characteristics. Some example systems can be adapted to current transmission CT geometries and exposures.

### Basic Example Providing Phase Contrast Detection

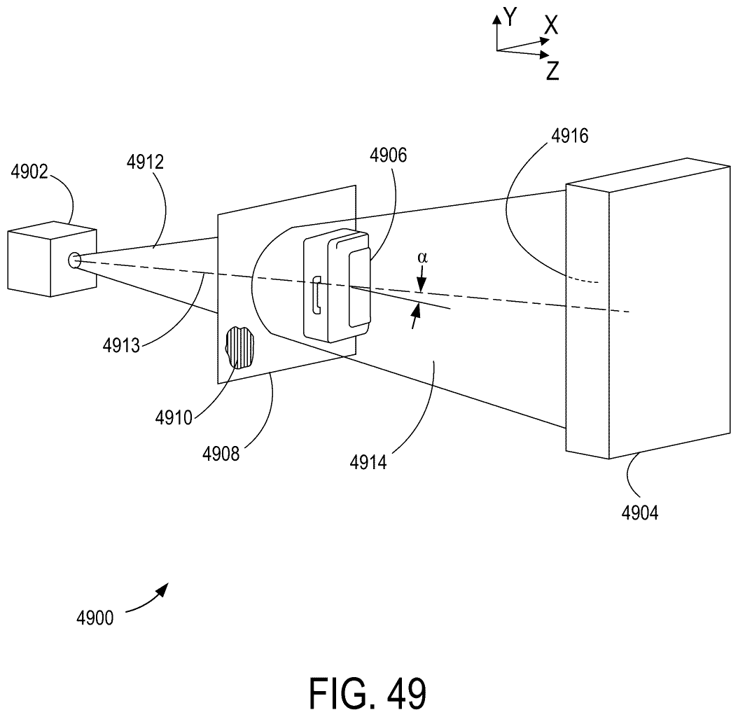

An example GBX system 4900 is shown in FIG. 49, which is similar in some respects to the system shown in FIG. 1A. Similar to currently available imaging systems, the GBX system 4900 can include an x-ray source 4902, an imaging detector 4904, and an object 4906 placed between them. A patterning element (grating/grid) 4908 can be situated near the object 4906. In many examples, the patterning element 4908 can be a grating or grid that includes an arrangement of parallel line elements 4910. During operation, the x-ray source 4902 can be configured to direct a beam 4912 of x-rays generally along an axial direction 4913 through the patterning element 4908 and object 4906 so that that a transmitted beam 4914 of x-rays can be detected by the detector 4904. The detector 4904 can be situated some distance back from the object 4906, e.g., rather than directly next to it, so that it can be positioned to detect a projected pattern 4916. The projected pattern 4916 on the detector 4906, in the absence of an object 4906, can be measured directly. When the object 4906 is in position to receive the beam 4912 of x-rays, a change in intensity on the detector 4904 can be measured to produce an absorption image. Deflection of the beam 4914 caused by the object 4906 will distort the shape of the pattern 4916 on the detector 4904, resulting in a differential phase image, and is sensitive to electron density gradients in the object 4906. Deflection of the beam 4914 by object microstructure (density fluctuations which are small compared to the imaged pixel size) will result not in a deflection of the pattern 4916, but in a loss of pattern contrast or visibility, resulting in a dark field image. If these visibility changes can be distinguished from spectral changes which can also affect pattern visibility, then the visibility changes can be attributed to small angle x-ray scatter and thereby used to identify materials with microstructure that is below the resolution limit of an attenuation image, including materials such as powders or composites.

### System Design Variations and Effects on Sensitivity

Various GBX examples can include different approaches and variations to provide phase contrast or GBX imaging. For example, additional gratings may be used to enable the use of larger focal spots or lower spatial resolution detectors. System design characteristics can also strongly affect the sensitivity of the system to phase and scatter. Sensitivity of a GBX system is closely related to the smallest deflection angle which can be detected. For example, with reference again to FIG. 49, for differential phase, the deflection angle, α, is related to the change in the phase shift of the wavefront Φ(x, y) perpendicular to the direction of the beam 4912 as it passes through an object:

$$α = \frac{λ}{2 π} \frac{\partial Φ ( x , y )}{\partial x}$$

where λ is the wavelength of the x-rays. The minimum angle which can be detected by the detector 4904 depends on noise in the image, but is related to the angle α₀ formed by the patterning grid 4908 projected on the detector 4904, compared with the distance from the object 4906 or patterning grid 4908 (whichever is closer) to the detector 4904:

$${α}_{0} = \frac{{{p}^{′}}_{obj}}{{l}_{2}} = \frac{{p}_{obj} ( {l}_{1} + {l}_{2} )}{{l}_{1} \cdot {l}_{2}}$$

where ρ_{obj} is the object grid period, p′_{obj} is the projected object grid period on the detector, l₁ is the distance from the source 4902 to the object grid 4908, and l₂ is the distance from the object grid 4908 to the detector 4904. More generally, l₂ can be the distance either from the object grid 4908 or the object 4906 to the detector 4904, whichever is smaller, and the object 4906 and object grid 4908 can be placed in either order. For convenience of understanding, the following analysis assumes the object 4906 is positioned upstream of the object grid 4906. At fixed energy, reducing the size of the projected pattern 4916 or increasing the distance from object 4906 to detector 4904 will decrease α₀, meaning that smaller deflections will be more readily apparent. If energy is changed, lower and less penetrating energies (longer wavelengths) correspond to larger deflection angles as well as larger phase shifts, making the same phase gradients more detectable. Deflections of size α₀ or greater, however, will result in a condition known as phase wrapping, where the local phase shift can be difficult to detect. Techniques for phase unwrapping exist, but this ambiguity can place limits on the ability to quantify large phase shifts in a highly sensitive system.

For small angle scatter in the dark field image, the cross section for scatter (also known as the dark field extinction coefficient) at a single energy depends on microstructure size distribution in the object 4906 as well as the difference in refractive index between different materials in the object 4906. In the simple case of uniform microspheres of diameter D in a medium, the cross section is given by:

$${μ}_{sc} = \frac{3 {π}^{2}}{{λ}^{2}} f {\vert Δχ \vert}^{2} {ξ}_{corr} \{ \begin{matrix}{D}^{′} - \sqrt{{D}^{′2} - 1} ( 1 + \frac{1}{2 {D}^{′2}} ) + ( \frac{1}{{D}^{′}} - & for D > {ξ}_{corr} \\ \frac{1}{4 {D}^{′3}} ) ln \vert \frac{{D}^{′} + \sqrt{{D}^{′2} - 1}}{{D}^{′} - \sqrt{{D}^{′2} - 1}} \vert , & \\ {D}^{′} , & for D \leq {ξ}_{corr}\end{matrix}$$

$$where {ξ}_{corr} = \frac{λ {l}_{2}}{{{p}^{′}}_{obj}}$$

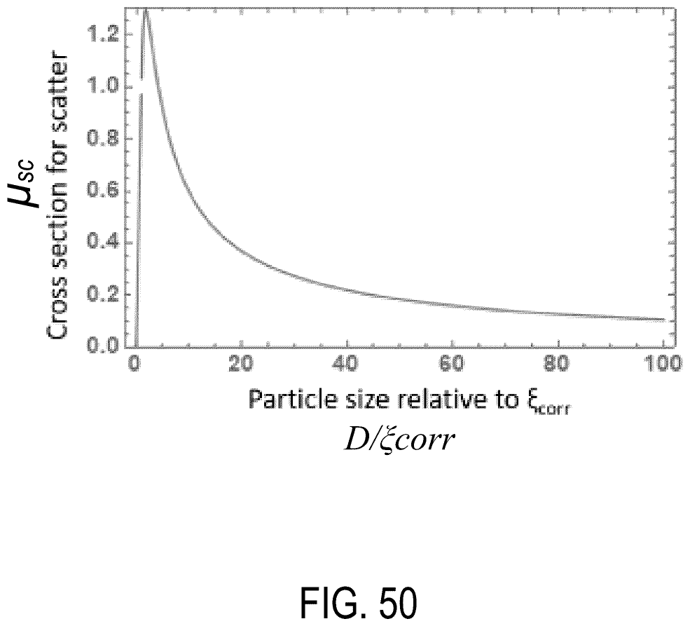

Here, the x-ray wavelength is λ, |Δχ| is the difference in the complex refractive indices of the particles and the medium, f is the volume fraction of the particles, and D′ is the ratio of D to the autocorrelation distance ξ_{corr}. The autocorrelation distance describes a characteristic length scale for detection of scatter, and is a function of l₂, the distance from the object grid 4908 to the detector 4904, and p′_{obj}, the projected period of the object grid 4908 on the detector 4904. The autocorrelation distance expresses the connection between the design of the gratings system and the ability to detect small angle scattering due to a given feature size. This can define the momentum transfer which is most readily detected as a change in fringe visibility. The scatter cross section in dimensionless units at a single energy is plotted as a function of D′ in FIG. 50.

As can be seen from FIG. 50, the cross section rises rapidly for very small particles and is peaked at about 1.8 times ξ_{corr}, indicating a characteristic particle size to which the system is most sensitive. For larger particles, the response falls off gradually, with some response remaining even for particles two orders of magnitude larger than ξ_{corr}. For the various example systems described herein, the autocorrelation distance is around 50-150 nm. The microstructure of interest for explosives detection is much larger than 50-150 nm, e.g., in the range of microns to 100's of microns, meaning that increasing ξ_{corr} will increase sensitivity. Note that ξ_{corr} is closely related to the characteristic angle for detecting phase shifts, α₀:

$${ξ}_{corr} = \frac{λ {l}_{2}}{{{p}^{′}}_{obj}} = \frac{λ}{{α}_{0}}$$

indicating that, for both phase and scatter, decreasing α₀ either through smaller pattern sizes at the object grid 4908 or through longer standoff to the detector 4904 will drive increased sensitivity at a fixed energy. Lower energies (longer wavelengths) also increase sensitivity for both phase and scatter. For a given object grid period (e.g., the spacing between the parallel line elements 4910), sensitivity is maximized when the grid 4908 is placed halfway between source 4902 and detector 4904.

### System Examples for Explosives Detection

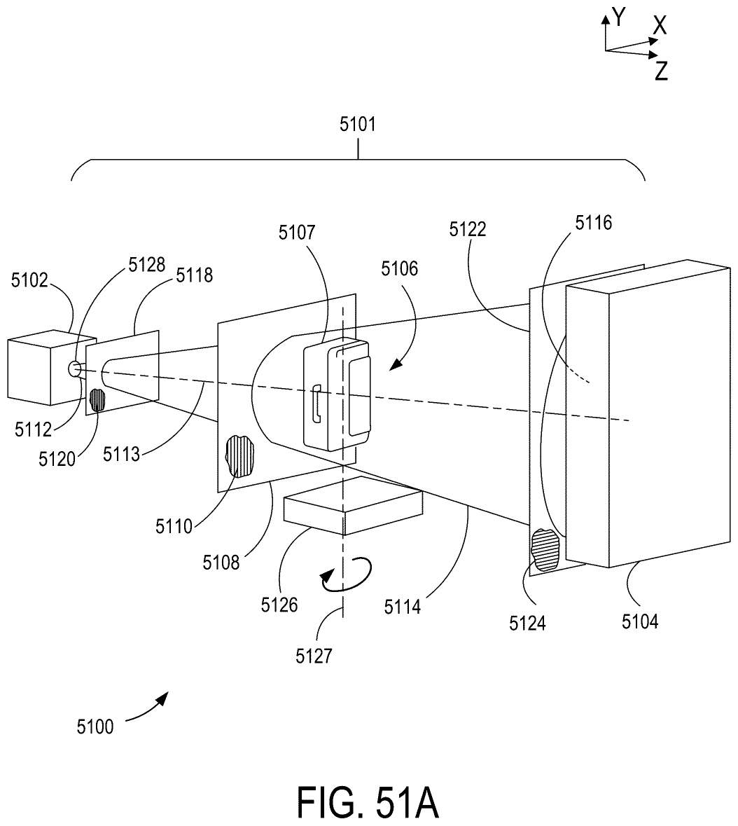

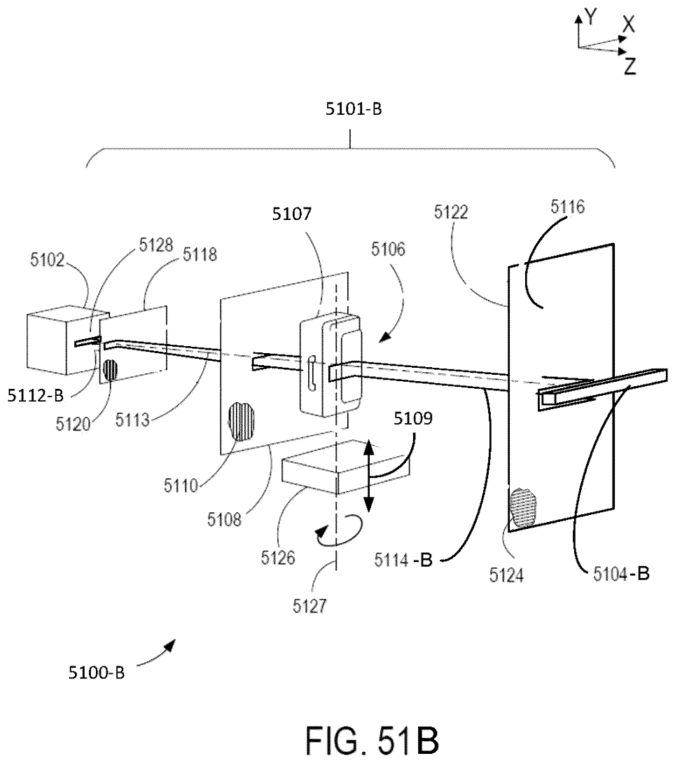

Example phase contrast imaging systems configured for explosives detection can include various additional components or modifications. For example, while decreasing pattern size can increase sensitivity, such a decrease can impose practical limitations both in the fabrication of grids, which becomes much more difficult as more penetrating x-rays are used (since more material is required to interact with the x-rays), and in sensitivity to vibration. FIG. 51A is an example GBX system 5100 that can be configured for explosives detection. The system 5100 includes an optical arrangement 5101 that includes an x-ray source 5102 and detector 5104 arranged to detect an object 5106 situated at an object placement location 5107 (also referred to as an imaging region). The optical arrangement 5101 can include a patterning element 5108 that can include grid elements, such as an arrangement of parallel line elements 5110. The patterning element 5108 can be referred to as an object grating or object grid and can be situated near the position of the object 5106. For example, the object grating 5108 can be situated approximately mid-way between the x-ray source 5102 and detector 5104 in many examples, which can be 0.4, 0.45, 0.48, 0.499, 0.5, 0.501, 0.52, 0.55, or 0.6 of this distance. The x-ray source 5102 can be configured to produce and direct an x-ray beam 5112 along an axial direction 5113 (e.g., along the z-direction) towards the object 5106. A transmitted x-ray beam 5114 that has passed through the patterning element 5108 and object 5106 can propagate towards the detector 5104. The detector 5104 can detect the x-ray beam 5114 to provide image data associated with the transmission of the x-ray beam 5114 through the object 5106, object grating 5108, and other components of the optical arrangement 5101 where present. Based on the optical arrangement 5101, the image data can include a plurality of detection modes, or signatures, including absorption, scatter, and/or differential phase.

At low energies (usually below 100 kV), higher sensitivity can be obtained using object gratings that produce an x-ray interference pattern at the detector 5104. Producing an interference pattern relies on a lateral coherence of the x-ray beam 5112 that is larger than the period of the pattern. For example, many systems construct patterns with periods on the order of a few microns. Because it can be difficult to directly detect these fine patterns, an analyzer grating (e.g., a grating and arrangement similar to analyzer grating 2920) can be used at the detector 5104 with an absorption pattern matched to the period of the undistorted interference pattern. Higher energy x-rays are more penetrating than lower energy x-rays and thus benefit from a grid of increased thickness to modulate the beam, while the lateral coherence requirement favors very fine patterns. The result of these competing effects can push fabrication requirements for grids to extremely high aspect ratios (ratio of the thickness of the grid to the grid spacing), as much as 100:1, making fabrication both difficult and expensive, if even possible. Further, micron-scale patterns will require sub-micron stability over the course of a measurement, which is operationally difficult.

Alternative configurations can be used in which the patterning element 5108 is absorbing rather than phase shifting so that lateral coherence is no longer a requirement to resolve a produced pattern 5116 at the detector 5104. Resolving the pattern 5116 can become easier as the pattern period 5116 is increased. Also, larger patterns can be advantageous for high energy applications because the grids used to produce them are more readily manufactured at thicknesses which can modulate a more penetrating x-ray beam (and in many cases can be purchased commercially), and because they reduce stability requirements proportionally with their size. As with interference-based patterns, an additional grid may be used at the detector 5104 to allow indirect detection of fine patterns (such as an analyzer grid 2920). Thus, some disclosed CT examples can use Moire-based detection. For example, some Moire-based CT examples can use an analyzer grid in a serial scanning Moire configuration, e.g., as described in U.S. Pat. No. 11,639,903 (incorporated by reference, as discussed above) such as in FIG. 29 et. seq. of U.S. Pat. No. 11,639,903 and related text. In some of such examples, a Moire image set can be collected at each rotational CT position, e.g., by providing a relative translation between the object and object grid to provide detection of x-rays through an object grid region and an adjacent object grid-free region. Further, some Moire-based CT examples can use an analyzer grid in a 2-D scanning configuration, e.g., as shown in FIGS. 29-48D herein and described hereinabove. In some of such examples, a Moire image set can be collected at each rotational CT position, e.g., by providing a relative movement between the analyzer grating and the object and detector gratings to different phase positions.

An additional grid 5118 may be used at the source 5102 to boost resolution of the (periodic) object grid 5108. The source grid 5118 includes pattern elements 5120, such as an array of a parallel line elements, which in many examples can be arranged parallel in relation to the parallel line elements 5110 of the object grid 5108. A grid 5122 can be situated adjacent to the detector 5104 and can be used with the object grid 5108 for direct imaging. For example, the proximity can be immediately adjacent or in contact, spaced apart by a distance sufficient to interpose one or more gratings or other components, or spaced apart by further amounts that remain substantially smaller than a distance between the object placement location 5107 to the detector 5104. Example ratios distances from the object placement location 5107 and detector grid 5122 to the detector 5104 can include 1000:1, 100:1, 50:1, 10:1, etc., and can depend on the types of object and modalities that are detected. The detector grid 5122 can include pattern elements 5124 which can be an array of parallel line elements. In many examples, the parallel line elements of the detector grid 5122 can be arranged perpendicularly in relation to the parallel line elements 5110.

The system 5100 can further include a movement stage 5126 that can be positioned adjacent to the object 5106, e.g., immediately behind the object grating 5108. The movement stage 5126 can be configured to rotate the object 5106 about a rotation axis 5127 to place the object a multiple rotational positions to collect phase contrast image data at each position. As shown, grating elements 5110 are generally parallel to the rotation axis 5127, though it will be appreciated that non-parallel arrangements are also possible. In many examples, the movement stage 5126 can be coupled to the object 5106, e.g., in connection with a conveyer belt, and the optical arrangement 5101 can remain fixed. In further examples, the optical arrangement 5101 can be rotated about the object 5106. In still further examples, both the optical arrangement 5101 and object 5106 can be rotated.

In a particular experimental example, the x-ray source 5102 was a standard system as is commonly used for non-destructive inspection (Comet MXR-160HP). Endpoint energy can be adjusted up to 160 keV, with a tungsten target that has an 11° target angle, a 40° take-off angle, a spot size of 1.0 mm or 0.4 mm, and a 0.8 mm thick beryllium window. The detector 5104 was a Teledyne RadIcon Shadobox 6k and was placed approximately 2 m from the x-ray source 5102. The three grids 5108, 5118, 5122 were used for the measurement in the experimental example, with each being a commercial antiscatter grid manufactured by Kiran and made with intervening lead and aluminum. The object grid 5108 was centered between the source 5102 and detector 5104 and had a period of 121 μm (210 lines per inch) with a 12:1 aspect ratio. To resolve these lines with a 1 mm focal spot source, the source grid 5118 was placed near an output window of the x-ray source 5102, with period 242 μm (103 lines per inch) and a 12:1 aspect ratio. The source grid 5118 can be operable to break the source beam 5112 into multiple line sources, each of which produces a clear pattern of the object grid 5108 on the detector 5104, offset by a single period. For dark field measurements, a method is needed to separate pattern visibility loss due to scatter from pattern visibility loss due to spectral changes (beam hardening). For this, the detector grid 5122 can correspond to an additional anti-scatter grid that is placed directly in front of the detector 5104, rotated 90° compared to the object grating 5108. This detector grid 5122 had a period of 242 μm and an aspect ratio of 6:1, such that the attenuation profile matches the attenuation profile of the object grid 5108. The visibility of detector grid 5122 changes with the incident spectrum, but not scatter in the object 5106, allowing for the visibility reduction in the object grid 5108 to be corrected for spectral effects. The detector pixels were 49 μm, with a Gadox scintillator, allowing for the projected grid patterns 5116 to be resolved directly (approximately 5 pixels per period). It will be appreciated that the listed parameters for the experimental prototype were configured for ease of use and testing in a lab environment, and that the parameters of deployed GBX systems can be different. Thus, as shown, the source grid 5118 can be used to boost visibility of the object grid 5108 while using a large focal spot source, and the detector grid 5122, oriented 90° relative to the source and object grids 5118, 5108, can be used to correct the dark field image for spectral changes. The object and detector grids 5108, 5122 are imaged directly on the detector 5104.

FIG. 51B depicts an alternative example GBX system 5100-B with an optical configuration 5101-B which is similar in many respects to system 5100 of FIG. 51. However, three primary differences from system 5100 can include: (1) the beam 5114-B in 5100-B from source 5112-B is collimated and has a smaller ratio of height or thickness along the axis of rotation, i.e., the Y-axis, relative to the width or spread of the beam along an X-axis (i.e., the axis that is both perpendicular to the Y-axis and to the beam path (i.e. the Z-axis)), (2) the detector 5104-B in 5100-B also has a smaller ratio of height, thickness, or pixel count along the axis of rotation relative to the width or spread of the beam along the X-axis, and (3) a second stage, or translational functionality 5109, is included in the system that allows for relative translation of the object 5106 or subject being examined relative to the X-ray exposure and detection system. In some variations the second stage may be part of or independent of the movement stage 5126.

The system 5100 of FIG. 51A is capable of gathering sufficient data for tomographic computation in a single set of rotational exposures while the system 5100-B of FIG. 51B would typically use a plurality of rotational scans that are offset in Y to provide sufficient data for a full object tomographic reconstruction. While the system 5100-B of FIG. 51B may have the disadvantage of longer scan time it provides the advantages of reduced detector cost and/or reduced X-ray scatter artifacts.

In the system of FIG. 51B, the beam height is preferably not significantly greater than that effective detector height to minimize scattering artifacts while the detector 5104-B preferably provides a sufficient number of pixels to effectively distinguish a full pattern from one successive grid line to an adjacent grid line. In the embodiment of FIG. 51B the elongated dimension of the detector 5104-B extends in a plane perpendicular to the Y-axis. Both an object grid and detector grid may be used with perpendicular relative orientations which may be different from that shown in FIG. 51B.

In some variations of the embodiment of FIG. 51B, the beam and the detector may be reoriented such that their elongated axes are oriented along the Y-axis and a conveyor like 2906 of FIG. 29 may be used to increment the object and optical arrangement such that the whole object can be scanned by a combination of rotary and translational movements.

### Computed Tomography Measurement and Reconstruction Experiments

A series of measurements was conducted to test the efficacy of the GBX system 5100 in performing CT measurements using high energy x-rays. The movement stage 5126 was positioned immediately behind the object grating 5108 and rotated the object 5106 to a plurality of computed tomography imaging positions. Source-object distance was 90 cm and source-detector distance was 198 cm. The field of view was determined by the dimensions of the detector 5104 (10×15 cm) and a ˜2× sample magnification. The spectrum used for CT acquisitions was 100 kVp with 2-mm Al filtration at 14.0 mA. To reduce the width of the beam 5112 and extraneous room scatter, a collimator 5128 of 1.25-cm thick tungsten with a 2-cm diameter opening was attached to the source 5102.

An initial calibration was performed using a pair of point-like objects (metal balls) to determine the relative orientation of the detector 5102, beam 5112, and rotation plane at the object 5106. The rotation plane can generally correspond to a plane perpendicular to the rotation axis 5127. For CT acquisitions, projections were collected between 0 to 360° in 0.5° steps, for a total of 720 acquisitions. Exposure time was 10s/frame, for a total exposure time of approximately 2 hours. Including overhead for stage motion and detector readout, the total measurement time was 2.5 hours per dataset. This relatively long acquisition time was driven in part by the relatively high spatial resolution (˜250 μm voxels) at which the measurement was conducted, and by the aim of having low-noise measurements for examining signatures. Dark and flat images were acquired prior to setting up gratings and used to correct for detector response variations. Reference images with grids present (“grid flat”) were acquired both before and after each CT dataset. Different samples were used to highlight the different contrast modes, including a scatter step wedge consisting of ZnO particles embedded in epoxy to test scatter reconstructions, a set of plastic rods of different materials to highlight sensitivity to different density and compositions, and a 3-D printed object to illustrate structural complexity.

Multiple measurement sets were taken, and systematic errors and noise were reduced over the course of acquisitions. After initial measurements with short acquisition times were found to be noisy, integration time was increased. After the random noise was reduced, systematic effects became more prominent in the data. In particular, projection images from the source 5102 and source grating 5118 combination showed low-frequency intensity modulations from left to right across the field of view, due to varying numbers of source grid slits 5124 illuminating each point on the detector 5104 (e.g., some points in this geometry are illuminated by 4 slits, and others by 5). In general, these artifacts are not visible after correction with a reference image. However, over the course of a longer measurement series, the quality of the corrected images was found to have decreased with time, with the source grid lines 5124 varying but typically becoming more prominent. No similar changes in the projections 5116 produced by the object grid 5108 or detector grid 5122 were observed.

Because of the placement of the source grid 5102 near to the X-ray spot, magnification is very high. This makes projections of features from the source grid 5118 particularly sensitive to any instability of the focal spot, either in size or location. The instability was observed to be greatest at the beginning of a CT acquisition and stability would increase as the acquisition continued, becoming noticeably more stable after about 2 hours. Therefore, for each of the CT acquisitions the source 5102 was operated for two full hours before starting the CT acquisition, which greatly reduced source instability artifacts. Some artifacts remained even after stabilizing the source 5102 with a warmup. For the final set of data collection, correction algorithms were developed which combine reference images (grid flats) from both the beginning and end of the acquisition to match the source grid artifacts for each frame in the CT dataset, discussed further hereinbelow. This type of artifact can be specific to the spot-size and source grid aspect ratio combination in use for these measurements and is unlikely to affect a faster measurement. However, it does illustrate the overall importance of stability for GBX and particularly GBX-CT systems.

Bad pixels can be corrected by interpolating using values from surrounding pixels, similar to that shown in FIGS. 21A-21B hereinabove. For example, because gratings-based phase and scatter extraction relies on high-frequency Fourier components, a simple linear interpolation of the values of surrounding pixels onto a bad pixel gives poor results. Instead, the surrounding pixels are fit to a linear combination of sinusoid patterns corresponding to harmonics of the grid frequencies, including a zero-harmonic constant value. The value of the bad pixel is then interpolated using this fit.

After image processing, vertical artifacts can be visible on the phase and scatter images. These artifacts can be caused by slight changes in the projection location of source grid artifacts over time, likely caused by changes in the beam spot (as discussed hereinabove). As the projection of the grating will have shifted from the pre- or post-exposure grating-only image, this can lead to artifacts during the phase and scatter extraction steps. To correct for these artifacts, a strip at the top of the radiograph with no object present was identified. This allowed an object-free region against which the pre- and post-exposure grating only images could be compared. The reference strip in the object image and the grating images is Fourier transformed. A region in Fourier space around the source grid oscillation peak is isolated and the Fourier components of the object image in that region are fit to a linear combination of the corresponding components of the grating image. This linear combination of the grating images is taken as the reference grating image to use during processing to extract the phase and scatter images.

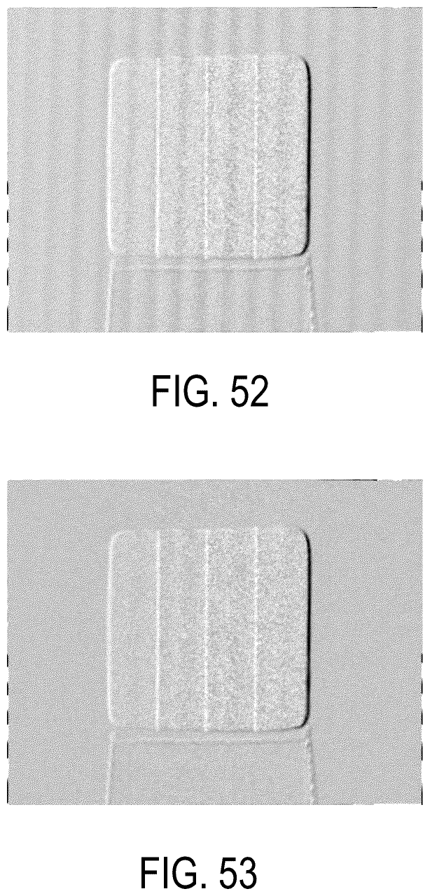

This technique has proven successful at removing the source grid artifacts. However, it leaves a non-zero baseline phase. In addition, there is a slight gradient to the phase across the image. This can be corrected out by averaging the phase over regions in each corner where no object is present. These averaged corner values are used in a bi-linear interpolation of the baseline phase across the image. This interpolated baseline phase is then subtracted off, leaving a phase image with values that are within the noise of zero in regions of the image away from the object. The outcome of this source grid artifact correction is shown in FIGS. 52-53, with FIG. 52 showing the phase image of the scatter step wedge object without correction and FIG. 53 showing the phase image of scatter step wedge object with correction. The vertical lines (artifacts) run approximately parallel to the axis of rotation for CT, and result in severe ring artifacts in CT reconstructions if not removed.

After artifacts in the projection images have been addressed, the absorption, phase, and dark field (visibility reduction) images are extracted from each projection image. The extraction process for directly-imaged periodic line pattern gratings is based on analysis of Fourier harmonics, and is described hereinabove in relation to FIG. 5. The dark field image is further normalized by a ratio with the dark field image based on the detector grating 5122, to isolate effects of structure in the object 5106 from spectral changes due to the object 5106, producing a scatter ratio image.

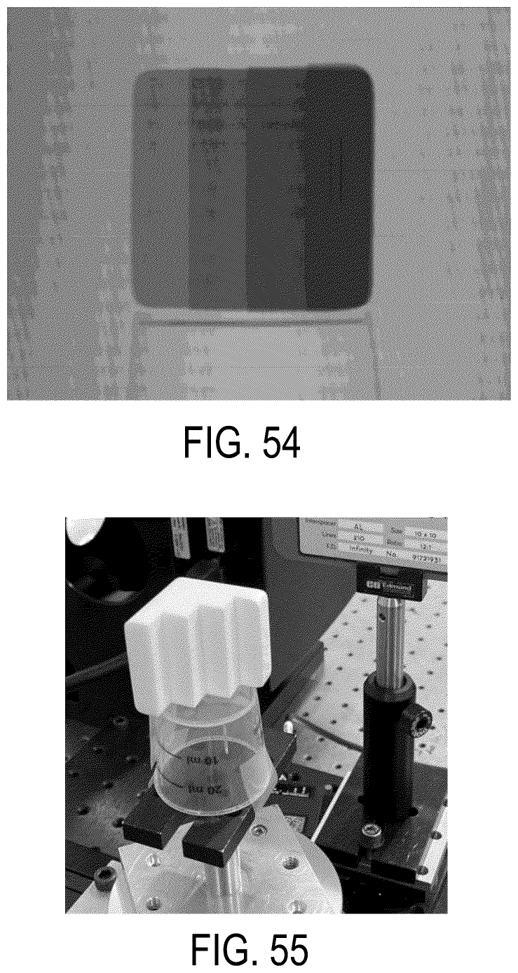

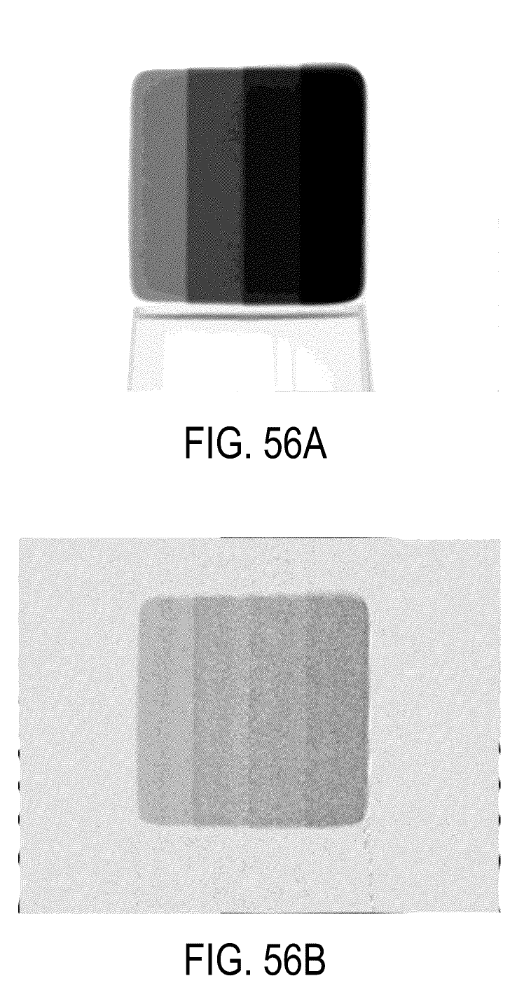

FIG. 54 shows an original radiograph of the step wedge object (ZnO particles, 5 μm and below in size, 20% weight fraction in epoxy) used in the experiment (corresponding to object 5106) and arranged at a single rotation angle. FIG. 55 is an image of the experimental setup. The image data shown in FIG. 54 has not yet been processed and the vertical and horizontal grid patterns are present. The zeroth harmonic extracted data (absorption) and the scatter ratio image (using the zeroth and first harmonic amplitude extracted data and after correction for spectral effects) for the step wedge object are shown in FIG. 56A-56B. As can be seen in FIG. 56A, the attenuation from the step wedge increases in steps from left to right, with darker regions corresponding to greater attenuation resulting from larger sample thicknesses. Further, the visibility measure shown in FIG. 56B shows that there is also greater small angle scatter in the thicker portions of the step wedge, corresponding to greater small angle scatter from a larger thickness of entrained zinc-oxide microspheres. Differential phase data was also extracted for this object.

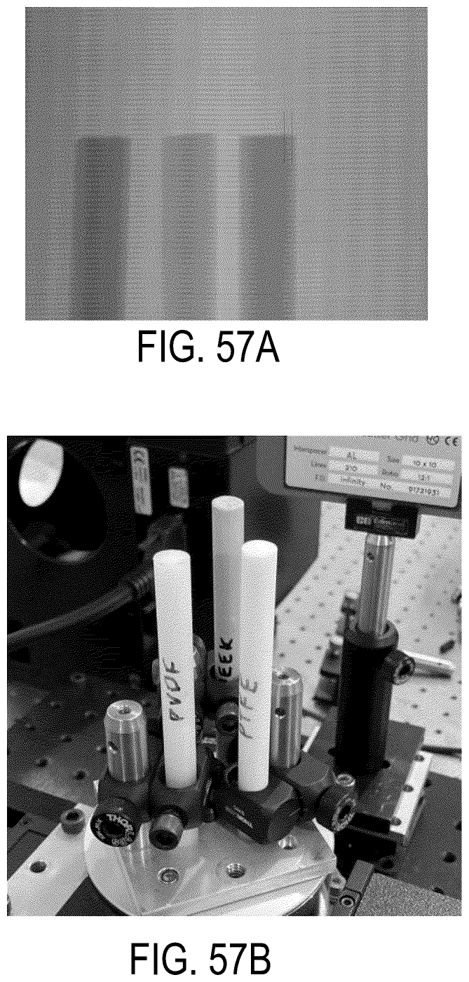

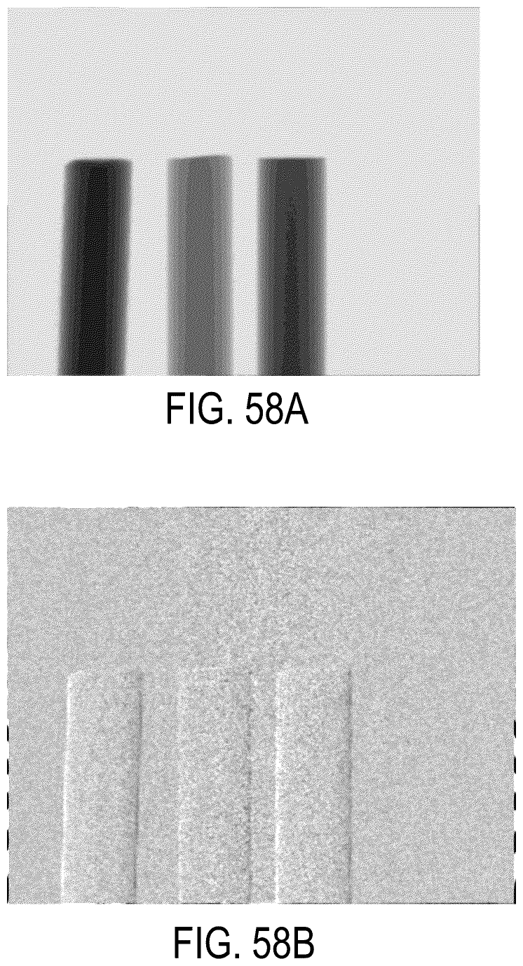

FIGS. 57A-57B show a radiograph of a plastic column object that was used to test the feasibility of making differential phase reconstructions and a picture of the plastic column object. It is composed of three plastic rods of ⅜″ diameter, more specifically, cylinders of polyetheretherketone (PEEK) (C₁₉H₁₂O₃ and 1.3 g/cm³), polyvinylidene fluoride (PVDF) (CH₂CF₂ and 1.77 g/cm³), and Polytetrafluoroethylene (PTFE) (C₂F₄ and 2.13 g/cm³). FIGS. 58A-58B show the extracted absorption information and the differential phase, respectively, for the plastic column object. In the differential phase image in FIG. 58B, the left-hand edges of the cylinders are bright (and positive), while the right hand are dark (and negative). This image can be interpreted as a differential image from left to right, where increasing object thickness from left to right results in positive values and decreasing object attenuation results in negative values. No scatter image is shown for these objects. Although pattern visibility is reduced behind the objects, they are internally homogeneous, and after correction for spectral effects no bulk scatter signal remains. A small apparent scatter signal may still be present at material interfaces which can be leveraged for tasks such as crack detection.

Performing a CT reconstruction requires that projection images can be described as the linear accumulation of a parameter as a function of sample thickness. For attenuation images (at a single energy), this condition is met by the negative log of the intensity image rather than the intensity image:

$$I = {I}_{0} {e}^{- \int μ ( x , y , E ) dt}$$

$$- ln \frac{I}{{I}_{0}} = \int μ ( x , y , E ) dt$$

For isotropic materials, scatter can be treated in an analogous fashion, but with initial and final visibility replacing intensity, and reconstructing on the scatter coefficient v(x,y,E). For both absorption and scatter, if sampled uniformly as a function of angle, an analytic reconstruction process known as Filtered Backprojection (FBP) can be used. In the parallel-beam approximation, each row of pixels is treated independently. A single row from each projection is extracted and stacked to form a sinogram, a two-dimensional image in which one dimension represents the position across the detector (pixel), and the other dimension represents the angle at which the projection was obtained. The sinograms are Fourier transformed, filtered using a ramp filter, inverse transformed and backprojected through the object space to form the reconstruction of a single slice through the object.

For the differential phase data, a different filter function can be used because the data is the differential of the image to be reconstructed. A signum function was used as the filter function and then the inverse Fourier transform and backprojection was completed. The signum function is defined as follows:

$$sgn x = \{ \begin{matrix}- 1 , & if x < 0 \\ 0 , & if x = 0 \\ 1 , & if x > 0\end{matrix}$$

Given the maturity of the CT field, software is readily available to make the reconstructions. For a relatively small detector active area placed 2 m from the source, a parallel beam approximation can be used for simplicity of reconstruction. Both absorption and scatter were reconstructed using FBP. For the differential phase reconstructions, different software is used to accommodate the modified filter function for the reconstruction. In the experiments, the ‘scikit-image’ python package was used, which contains a number of image processing methods including an inverse radon method, iradon, that was modified to complete filtered back projections with the signum function.

In general, the parallel beam approximation is not applicable to many geometries, particularly for security screening. Multiple software packages are available for CT reconstruction in other geometries. Tests were also conducted using the ASTRA toolbox which provides for fan and cone beam reconstructions using the FDK reconstruction method as well as iterative, model-based reconstructions which can be more flexible to different angular sampling schemes.

Scatter reconstruction can involve additional complexities. For isotropic materials, scatter is isotropic and can be reconstructed in the same manner as absorption. However, many materials, such as wood or other fibrous materials, are not isotropic. For these materials scatter is directionally sensitive, and is better represented as a tensor rather than a scalar value. The scatter tensor can be completely characterized by collecting multiple datasets with different directional sensitivity (either anisotropic object grid patterns or rotating the object grid), but in a single dataset this may lead to ambiguities. In some examples, this can be treated numerically.

It has also been found that the differential phase reconstruction using the signum filter can provide identical results to a process where each differential phase image was integrated first and then the set reconstructed using a ramp filter (as would be performed for absorption and scatter). In view of this equivalence, in some examples the alignment between the rotation axis and the grid lines of the object grating can be non-parallel.

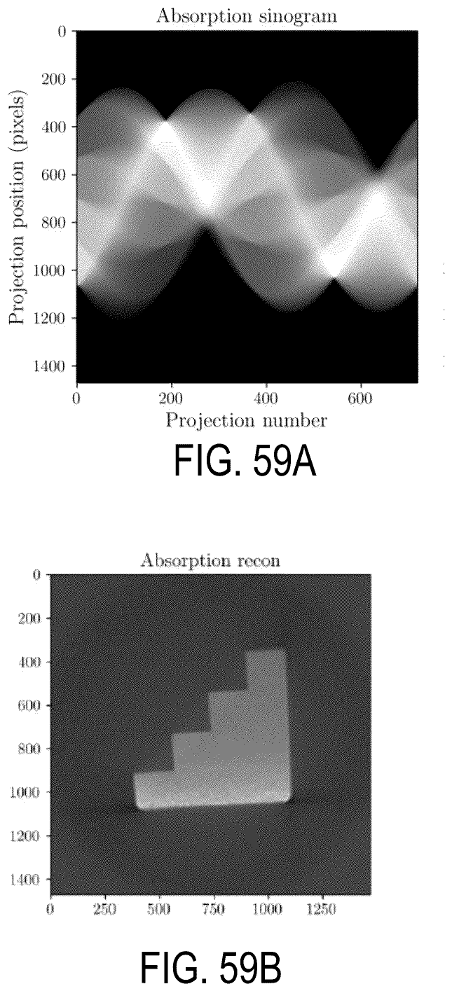

FIGS. 59A-59B respectively show an absorption sinogram for the scatter step wedge object, and the reconstruction using filtered back projection (FBP) and a ramp filter. Commercial CT software typically includes artifact and noise reduction (i.e., ring removal) to enhance CT reconstructions. In some examples, additional post-processing corrections can be made. The reconstruction shown in FIG. 59B was made for a single row of the image data, representing a single slice through the step wedge object. A greater attenuation at one end of the step wedge object can be seen in the reconstruction that is not evident in the radiograph. This is likely due to settling of the zinc oxide particles when the step wedge object was made.

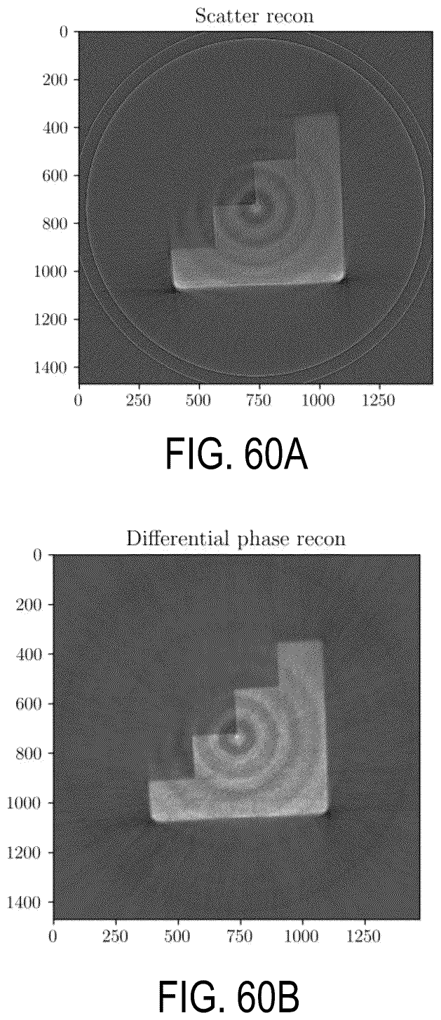

FIGS. 60A-60B show the reconstruction of the scatter and differential phase signals for the step wedge object. To lower noise, the scatter and differential phase reconstructions were made by averaging the image data over 100 rows and 10 rows of the projections, respectively. Thus, the reconstructions represent a slice of greater thickness than that reconstructed for the absorption data. In the scatter reconstruction, a greater scatter in the lower portions of the step wedge can be clearly seen, here correlating with where the higher attenuation was seen in the absorption reconstruction. The phase reconstruction also shows increased signal near the bottom edge where the ZnO concentration is higher, but the difference is less pronounced, consistent with the phase signal's dependence primarily on electron density, rather than on Z_{eff} or microstructure.

The scatter signal is reconstructed with the ramp filter while the differential phase signal is reconstructed with the signum function. Though the phase projection image is the differential signal, the reconstruction with the signum function shows a generally flat profile, demonstrating the phase integration incorporated into the CT reconstruction step. Some ring artifacts can be seen in the reconstruction images shown in FIGS. 60A-60B. Such artifacts can occur in CT when there is an intensity variation across a projection that is independent of the object, such as a variation in sensitivity that is not corrected properly. Here, the rings are due to low-frequency (and low intensity) variations from source instability remaining after the tube warmup and processing corrections discussed hereinabove. Although preprocessing to remove source grid artifacts has been employed for these images, post-processing ring removal methods were not employed. Numerous methods for ring removal are available, as understood in the art.

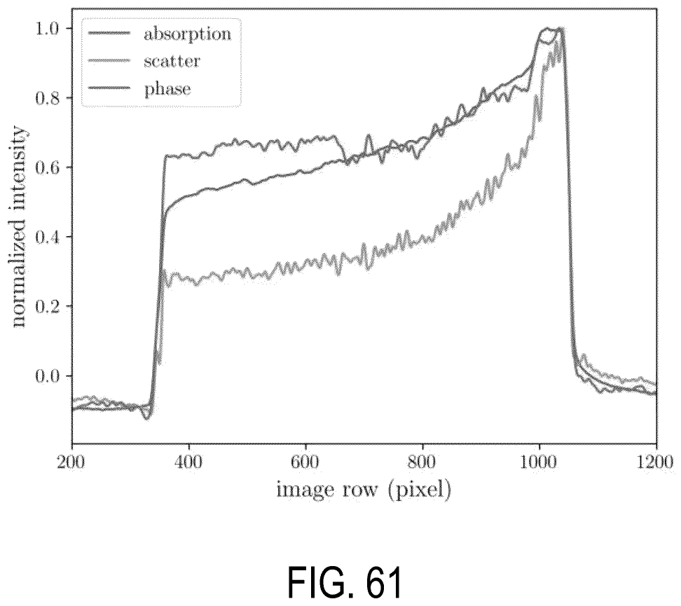

A graph in FIG. 61 shows the unique characteristics of each of the absorption, scatter, and phase signals. These normalized line profiles of the reconstructions through column 1000 indicate that the scatter signal is particularly sensitive to the higher density of the zinc-oxide particles. The phase reconstruction shows a flatter profile than both the scatter and absorption, indicating lower sensitivity to the zinc-oxide particles.

The relative signal strength of each mode can be considered by evaluating the Contrast-to-Noise ratio (CNR). This metric compares the signal difference between an object and background with the root-mean-squared sum of the noise, providing an estimate of detectability. CNR values are shown in CT Table 1 for the absorption, phase, and scatter reconstructions, for a region near the bottom right corner of the step wedge object. For this sample, CNR is highest in absorption, lowest in scatter, and intermediate for phase.

CT Table 1. Contrast-to-noise ratios for absorption,
phase, and scatter in the step wedge object.

| Contrast | |
|---|---|
| Mode | CNR |
| Absorption | 15.9 |
| Phase | 7.1 |
| Scatter | 2.3 |

The plastic cylinder data and reconstructions were also considered. As explained hereinabove, the plastic cylinders object included materials with different bulk density and electron density. PEEK, PVDF, and PTFE, which have densities of 1.3, 1.77, and 2.13 g/cm3, respectively. This corresponds to electron densities, ρₑ, of 0.68, 0.88, and 1.06 electron-mole/cm³.

FIG. 62A shows the sinogram for the absorption signal from the plastic cylinders object. FIG. 62B shows the reconstruction, which shows greater attenuation (brighter circles) for the plastics that have higher density, where PTFE (left circle in the reconstruction) has the highest density and PEEK (bottom circle) the lowest. The ability to make quantitative measurements of the attenuation can be confirmed with the absorption reconstruction by comparing it to known attenuation values from the x-ray data. Here, the attenuation values are weighted by the spectrum impinging on the plastics, simulated with SpekCalc based on an endpoint energy of 100 keV and attenuated by the 2-mm aluminum filter as well as an aluminum thickness corresponding to the beam passing through the interstitials of three grids. The result of this analysis is shown in CT Table 2. Expected linear attenuation coefficients, in cm1, are shown in the second column. For easier comparison with voxel values in the reconstruction, we normalize these values with respect to the attenuation coefficient of PTFE, shown the third column. Average reconstructed voxel values are shown in the fourth column, and normalized with respect to the PTFE values in the fifth column. After normalization, the measured material ratios are in good agreement with the ratios for the linear attenuation coefficients.

CT Table 2. Comparison of a weighted average of the known
attenuation coefficients, μ, to that extracted from
the reconstructed attenuation profiles shown in FIG. 62B.

| | μ weighted | | | Extracted μ |
|---|---|---|---|---|
| Compound | (cm⁻¹) | μ norm. | Extracted μ | norm. |
| PTFE | 0.4737 | 1.00 | 0.0329 ± 0.0002 | 1.00 ± 0.01 |
| PVDF | 0.3883 | 0.82 | 0.0273 ± 0.0002 | 0.83 ± 0.01 |
| PEEK | 0.2595 | 0.56 | 0.0188 ± 0.0002 | 0.57 ± 0.01 |

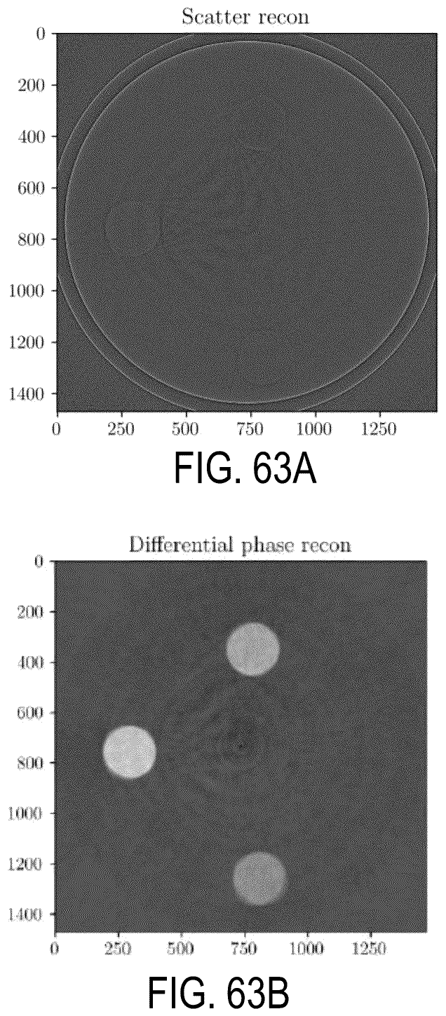

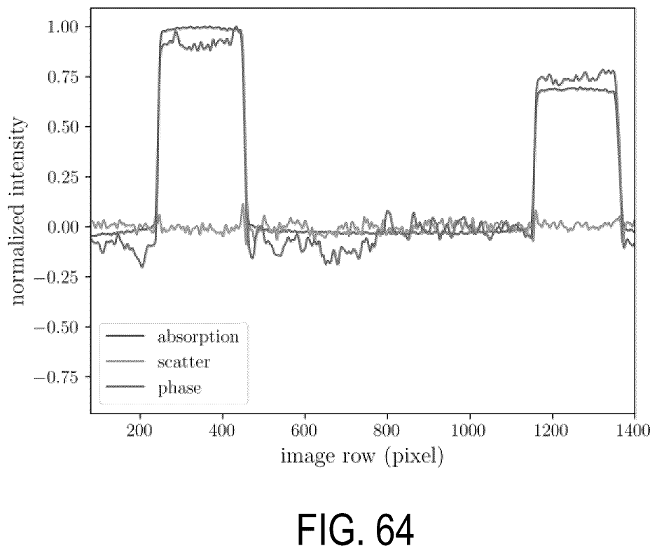

FIGS. 63A-63B shows reconstructions of the scatter and differential-phase signals, again averaging over 100 and 10 image rows, respectively, before reconstruction. The PVDF cylinder is at the top, PTFE at the left, and PEEK at the bottom. However, they are barely visible in the scatter reconstruction. Because the plastics of the plastic cylinders object have no entrained particles and are homogeneous, the scatter signal is consistent with zero except for small features at edges, unlike with the step wedge object. Significantly, this confirms that the beam hardening correction is allowing the separation of spectral effects from material microstructure. The reconstruction of the differential-phase data in FIG. 63B looks similar to the absorption reconstruction in FIG. 62B. However, there are quantitative differences seen in the line profiles of the reconstructions as shown in FIG. 64. The line profile is along column 790 of the reconstructions, through the PVDF and PEEK plastics.

The phase reconstruction is expected to be proportional to the x-ray index of refraction for the material, which is proportional to electron density. To explore this, regions of interest (ROI) were designed in the locations of each of the materials cylinders in the phase reconstruction. Each of these ROIs is averaged to find a measure of phase shift relative to background. The results are shown in CT Table 3. As with the attenuation properties, relative properties can be compared by examining the values in each region as compared with PTFE.

CT Table 3. A comparison of physical characteristics of the
plastics to the reconstructed differential phase values, where
the mean and standard deviation of a ROI are reported. The
electron density ρₑ and extracted phase are
normalized for direct comparison.

| | | ρₑ | ρₑ | Extracted | Phase |
|---|---|---|---|---|---|
| Compound | Z_{eff} | [mole/cm³] | norm. | phase | norm. |
| PTFE | 8.50 | 1.06 | 1.0 | 0.98 ± 0.04 | 1.0 ± 0.04 |
| PVDF | 8.01 | 0.88 | 0.83 | 0.78 ± 0.04 | 0.80 ± 0.04 |
| PEEK | 6.35 | 0.68 | 0.64 | 0.62 ± 0.03 | 0.63 ± 0.03 |

The relative phase values are in good agreement with the relative electron densities, independent of the average atomic number variations. The comparison of values extracted from the absorption and phase data, and their correspondence with the expected physical properties of the measured materials, demonstrate the quantitative information available in the phase data is physically distinct from attenuation at this spectrum. Contrast-to-noise values were also evaluated for the plastics. CNR is zero for scatter, because these samples do not have microstructure at length scales to which the system is sensitive. With all three materials, CNR is higher in absorption than in phase. Values are shown in CT Table 4.

CT Table 4. CNR values for the plastic rods.

| | CNR | CNR |
|---|---|---|
| Compound | absorption | phase |
| PTFE | 46 | 16 |
| PVDF | 57 | 14 |
| PEEK | 39 | 13 |

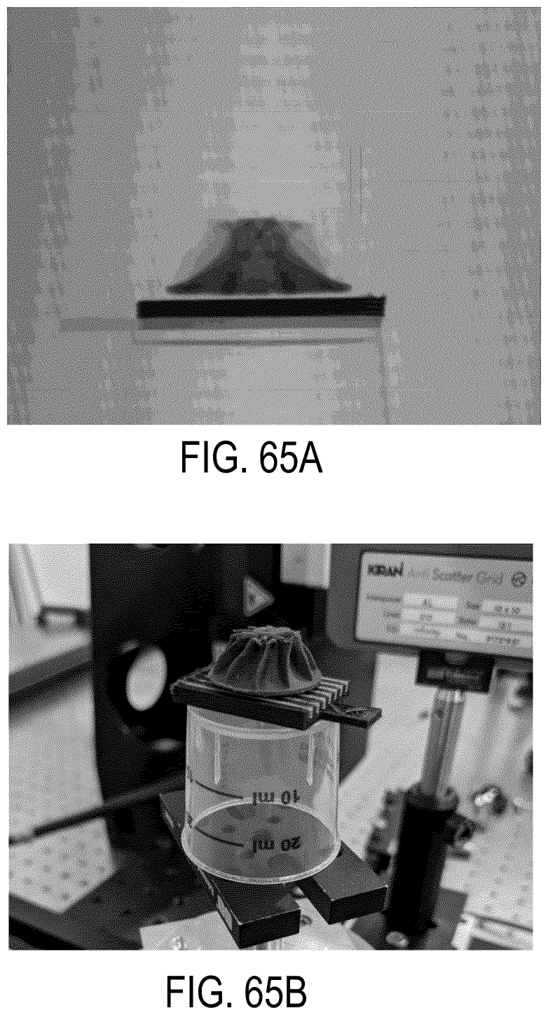

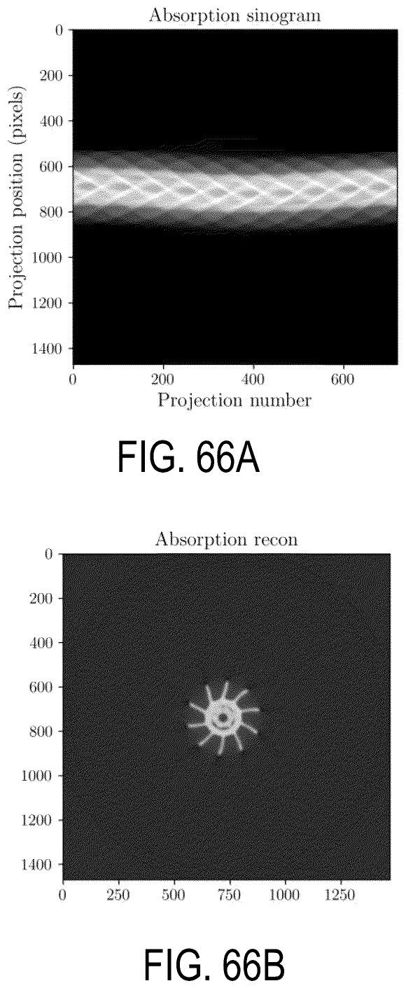

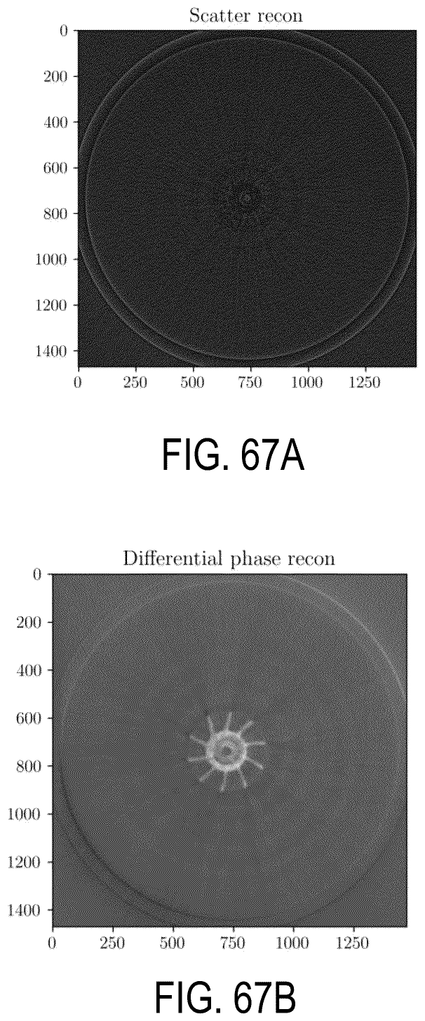

In addition to the step wedge and the plastics, a 3D-printed object was scanned. The radiograph and picture of the object are shown in FIGS. 65A-65B. The absorption sinogram and reconstructions of absorption, scatter, and phase are shown in FIGS. 66A-67B. Here, the fan-like structure of the object reconstructed in the absorption and phase reconstruction can be seen. For this object, no significant microstructure was detected in the scatter reconstruction.

The results shown hereinabove demonstrate the ability to reconstruct data from three contrast modes: absorption, phase, and scatter, and also that the different modes provide physically distinct signatures. Different characteristics of the GBX system can be tailored to the different modes, such as choice of source, detector, x-ray optics, and footprint, as well as sensitivity. The GBX signatures in the context of information currently in use for security screening (e.g., in aviation security) are typically electron density and effective atomic number derived from dual energy measurements. A representative CT geometry with a 75 cm diameter imaged region and a 1 m source-detector distance was used to consider the possible sensitivity of GBX system designs.

Computed tomography measurements are particularly useful for security screening because the 3-D reconstruction resolves the pathlength ambiguity that is present in projection imaging. When dual energy imaging is employed, this enables automatic threat recognition for many materials based on electron density and effective Z. The combination of absorption, phase, and scatter offers the potential to further enhance material discrimination. The absorption component of a GBX measurement offers equivalent material information as in conventional absorption imaging, with sensitivity to density and effective Z particularly for higher Z materials. The scatter reconstruction, when corrected for spectral effects, provides a measure of microstructure, enabling powders or composites to be distinguished from homogeneous materials such as liquids. This is a novel signature, not currently exploited in security screening.

The phase reconstruction, as seen hereinabove, is directly sensitive to electron density. It has been shown that the combination of phase and absorption can be used to parametrize density and effective Z in the same manner as dual energy. While this means that the phase measurement is not a physically independent measurement when compared with dual energy, phase still may provide advantages in terms of different systematic errors or higher contrast to noise. In several systems, extremely high contrast to noise has been demonstrated using phase contrast, detecting density variations of 1 part in 10⁶. As energy is increased, phase can still provide greater sensitivity to density differences than absorption. However, in both these examples, all materials were immersed in index matching fluid to avoid phase wrapping at material boundaries, which can be a limitation with high sensitivity systems.

As discussed hereinabove, the signal for phase and scatter depends on the geometry of the system and, in particular, on the characteristic scattering angle α₀ (for scatter, this holds when the microstructure length scales are significantly larger than the autocorrelation length). The geometric sensitivity (can be defined based on the ratio of the autocorrelation length of a proposed system to that of a baseline system, or equivalently the ratio of the inverse scattering angles. If the same x-ray spectrum is used in both cases, a simple expression can be obtained depending only on the geometry of the two systems:

$$ζ = \frac{( {l}_{2} / {{p}^{′}}_{obj} )}{{( {l}_{2} / {{p}^{′}}_{obj} )}_{baseline}} = \frac{1 / {α}_{0}}{{( 1 / {α}_{0} )}_{baseline}}$$

$$where$$

$${α}_{0} = \frac{{{p}^{′}}_{obj}}{{l}_{2}} = \frac{{p}_{obj} ( {l}_{1} + {l}_{2} )}{{l}_{1} \cdot {l}_{2}} .$$

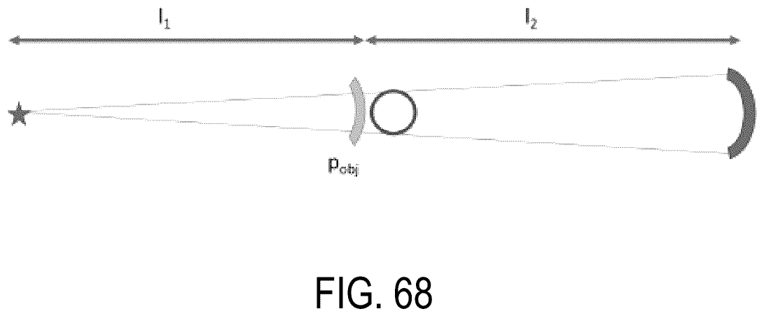

The geometric sensitivity parameter (can be used as a measure of the relative sensitivity to phase and scatter of different design configurations. For comparison, the system geometry from the measurements in the experiment discussed above was used as the baseline system. FIG. 68 shows the baseline system geometry, with the imaged region shown as a circle around the sample location. In this geometry, there is approximately 1 m from source (left most star) to the object grid (centered arc) and object, a small imaging region (shown here as a circle, with the axis of rotation perpendicular to the plane of the page), and approximately 1 m from object grid and object to detector (right most arc). For the lab geometry, sensitivity with a given object grating period (121 m) and fixed overall system length is maximized by placing the object grid midway between source and detector.

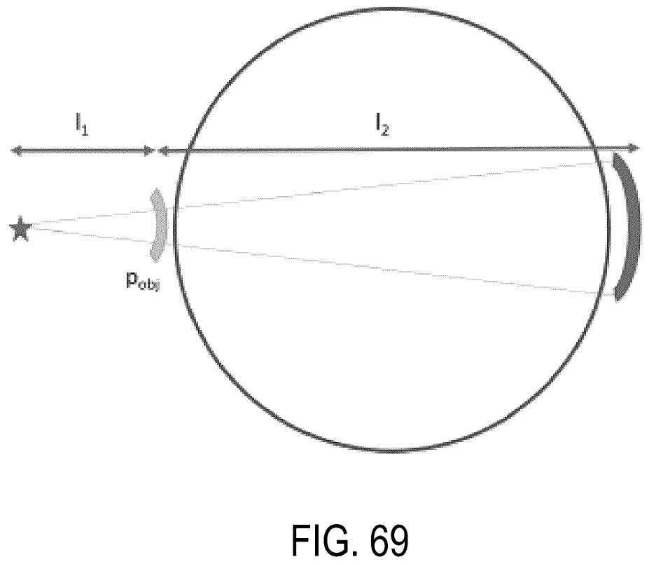

The nominal geometric parameters for a security screening CT can be significantly different than the baseline system described here. For example, the total system length (source-detector distance) can be around 1 m in many examples, rather than 2 m, which reduces the available detector standoff. Also, the imaged region can be very large in relation to the source-detector distance, e.g., encompassing a cylinder of approximately 75 cm diameter. As a notional geometry, the distance between the source and the imaged region can be approximately 20 cm and the distance from the detector to the imaged region can be κ cm. Under these conditions, the placement of the object grid can be more limited, e.g., the object grid cannot be placed midway between source and detector. In many of such examples, the object grid can be placed on the source side, as close as possible to the imaged region, as shown in FIG. 69. As can be seen in the figure, the axis of rotation is perpendicular to the plane of the figure.

Various GBX-CT system examples can be tailored based on sensitivity, fabrication difficulty, and complexity, with sensitivity results summarized in CT Table 5. As discussed hereinabove, pattern detection can be done directly, if the projected pattern sizes are resolved on the detector, e.g., resolved by at least 5 pixels on the detector. Indirect detection allows the use of finer grids and/or larger detector pixels. It relies on the use of an analyzer grating matched to the projected pattern, and stepping or scanning to extract absorption, differential phase, and dark field information for each pixel.

For direct imaging with a baseline grid, 121 m pitch object grid was used and placed as close as possible to the imaged region (e.g., l₁=15 cm, l₂=75 cm). The projected pattern size is 766 μm on the detector, which could be imaged directly with detector pixels as large as 150 μm. Geometric sensitivity (=0.25, compared with the baseline system. By direct imaging with a finer pitched object grid, a higher sensitivity can be obtained. For example, digital detectors with pixel size of 50 μm are readily available, corresponding to a minimum projected pattern size of 250 μm and an object grid period (placed at l₁=15 cm and l₂=75 cm) of 39 μm. This would require a source grid period of 47 μm. Grids finer than approximately 85 μm period are not available off the shelf and would require microfabrication as achieving the needed material thickness at these periods can be difficult. Finally, a decrease in grid period comes with a corresponding increase in required mechanical stability. In this finer grid configuration, geometric sensitivity can be improved to ζ=0.78 compared with the baseline system. With a very fine grid period, indirect imaging can be used and provide a sensitivity that can approach the sensitivity of the baseline system, as the object grid (and also the source grid) are made finer. For example, a source grid period of 30 μm, placed at l₁=15 cm and l₂=75 cm, could only be imaged directly with detector pixels 36 μm or smaller. Detectors as this resolution would be very costly or unavailable, and data readout rates could present a challenge. By using indirect imaging, the sensitivity in this case would be ζ=1.01 compared to the baseline system.

Another option for extending sensitivity while maintaining direct imaging (with the somewhat arbitrary choice of 50 μm pixels representing the smallest feasible detector pixel pitch) is to make the grids finer but to increase the magnification by moving the object grid closer to the source. In one example configuration, an object grid has a 4 μm period placed 1.5 cm from the source and a source grid having a 4.1 μm period produce a projected period of 250 μm, for a geometric sensitivity of ζ=0.89. However, this may be increasingly difficult to fabricate and include high precision constraints for a relatively small increase in sensitivity. Another alternative configuration that may be used in some examples is the placement of the object grid on the detector side of the imaged region, rather than the source side. For the sake of comparison, the imaging region can be assumed to be shifted closer to the source, such that the imaged region starts 5 cm from the source, and the distance from the imaged region to the detector is 25 cm. This may be unfavorable in practice due to the width of illumination from the source. In this case, any of the object grids discussed previously but placed with l₁=75 cm and l₂=15 cm would result in the same geometric sensitivity as the case where l₁=15 cm and l₂=75 cm. However, the projected grid pattern on the detector would be much smaller and may require indirect imaging in many or all examples. Also, the lateral size of the grid would have to be much larger. The analyzer grid used for indirect imaging would likewise have to be large in size and fine in frequency, while the source grid frequency would be coarser. Such examples may require custom fabrication of larger grids, may include issues with illumination angle, and may contain additional complexity overall.

CT Table 5. A summary of grid and geometry options explored in this
section. Some of the options show that sensitivity of the baseline
system could be reached within a nominal CT system geometry.

| | | | Detector | | |
|---|---|---|---|---|---|
| | Object | Source | resolution | | |
| | grid | grid | (μm) (if | | |
| | period | period | direct | | |
| Case | (μm) | (μm) | imaged) | ζₘₐₓ | Comments |
| 0. Baseline | 121 | 242.0 | 48.4 | 1.00 | COTS |
| 1. Baseline | 121 | 145.2 | 145.2 | 0.25 | COTS |
| grid, direct | | | | | |
| imaging | | | | | |
| 2. Finer grid, | 39 | 46.8 | 46.8 | 0.78 | grid |
| direct | | | | | fabrication |
| imaging | | | | | |
| 3. Very fine | 30 | 36.0 | 36 | 1.01 | grid |
| grid, indirect | | | | | fabrication |
| imaging | | | | | |
| 4. Very fine | 4 | 4.1 | 48 | 0.89 | grid |
| grid, direct | | | | | fabrication |
| imaging | | | | | |
| 5. Grid on | 121 | 726.0 | 29.04 | 0.25 | grid |
| detector | | | | | fabrication |
| side | | | | | (analyzer) |

If commercial anti-scatter grids are used, sensitivity in the GBX CT geometry will be lower than the lab system. However, this does not necessarily make the measurement infeasible. However, the reduced sensitivity does limit the quality of the data produced. Other options for increasing sensitivity represent more difficult grid fabrication issues.

As discussed above, another important parameter in GBX systems is stability. As can be seen in the lab measurements, where system stability is maintained throughout a CT measurement, the reconstructions that are produced contain fewer image artifacts. Currently available CT scanners image with resolution on the order of ˜1-3 mm, and so stability requirements for GBX systems can be expected to approximately on that level or a somewhat below. When gratings are added, the length scale to which the system must be held stable drops to approximately ⅕ of the grating period. This means that even in the case of the commercial anti-scatter grating, care must be taken with the engineering design to achieve the needed stability and systems with finer grid spacings pose a greater difficulty.

Another significant difference between security screening CT and the GBX CT performed in the lab is the size of the imaged region, particularly relative to the total system length. At a minimum, this means that a parallel beam assumption for the reconstruction geometry will not be appropriate. This should not be problematic, because methods exist for reconstructing a range of measurement geometries. One characteristic specific to GBX systems is that the sensitivity of the system to angular deviations decreases linearly with distance between a point in the object and the object grid. This means that the same region of the object exhibiting a large phase or scatter signal when close to the grid will show a reduced signal at all other rotation angles. This sensitivity gradient across the imaged region violates the normal properties for CT reconstruction, and may lead to reconstruction artifacts and decrease average sensitivity.

The usefulness of a given GBX CT system in improving material discrimination can depend on the ability of the system to provide material properties in a fixed amount of time. For example, lab-based measurements can provide material information, but at much longer timescales than desired in a security setting, and with a significantly different system geometry. A useful metric for describing the detectability of a feature in an image is the contrast to noise ratio (CNR):the difference in signal between the object and the background, compared with the square root of the sum of the squared errors. If an imaging standard is measured on different systems or under different acquisition conditions, CNR can provide a means to compare system image quality. CNR is shown below for visibility reduction (a scatter image), but a similar expression can be used for any of the non-differential images or image reconstructions.

$$CNR = \frac{{[ \frac{V}{{V}_{0}} ]}_{bkgd} - {[ \frac{V}{{V}_{0}} ]}_{obj}}{\sqrt{{σ}_{bkgd}^{2} + {σ}_{obj}^{2}}}$$

For a given object, contrast in conventional absorption or the absorption mode of GBX is primarily a function of x-ray spectrum, although background scattering can also affect contrast. For phase and scatter, contrast is also affected by the geometric sensitivity of the system, as discussed hereinabove. For small variations, this can be approximated as linear, although linear approximation is generally not appropriate with phase wrapping in the differential phase image, or for particle sizes of the same order of magnitude or less than the correlation length for the scatter image. Error in the image value can be estimated by measuring the standard deviation of a group of pixels in the same region. This can correspond to an estimate for how much image-to-image variation might be seen due to random noise. For absorption, random noise can be largely a function of the total exposure: counts per pixel for the given exposure time. For phase and scatter, noise can be a function of both total exposure and of the visibility of the object grid pattern, because the pattern visibility gives an estimate of the fraction of the beam which is modulated and can be used for extracting phase and scatter. In either case, increasing exposure is expected to reduce noise according to counting statistics, with a 4× increase in exposure resulting in a 2× decrease in noise.

Lab measurements were conducted at 7200 seconds acquisition, with 2m from source to detector and a reconstructed voxel size of 200 m on a side. Although this acquisition time is extremely long relative to operationally relevant measurement times, the spatial resolution is also higher than needed. Increasing the voxel size to 2 mm can give a 1000× increase to voxel volume. In principle, this should enable a 1000× decrease in measurement (exposure) time resulting in the same number of counts per voxel, corresponding to 7 seconds. In practice, reducing measurement time can involve additional factors, such as stage motions and detector readout time. Sensitivity or signal also can present challenges. For example, by using similar equipment to the lab measurement but working with the security screening geometry, sensitivity is expected to drop by a factor of 4 (ζ=0.25). Given the sensitivity gradient across the imaged region, the average geometric sensitivity will be further reduced. Assuming reconstruction is still possible, this would be roughly ζ=0.13. In terms of CNR, the source-detector distance of the security screening system is half that of the lab, for a 4× exposure increase and a 2× noise reduction, partially offsetting the decrease in sensitivity. Further reductions in spatial resolution (voxel size) or increases in exposure could result in CNR levels sufficient for threat detection. However, a limitation to CNR as a metric for detection is that it only accounts for statistical noise. Systematic errors, such as ring artifacts in reconstructions, image artifacts from system instability, or “cupping” artifacts resulting from beam hardening or Compton scatter eventually limit detection ability even as exposure is increased. This presents a limitation to offsetting arbitrarily low geometric sensitivity with increased exposure per voxel.

GBX-CT systems offer the potential of enhanced materials discrimination, e.g., based on the combination of absorption, phase, and scatter contrast combined with the ability to unambiguously identify volumetric regions of material. Absorption and phase provide information on the same physical signatures as dual-energy absorption, but the systematic errors and the accuracy of the information extracted may be significantly different. Scatter provides material microstructure information. With disclosed examples, GBX-CT measurements have been demonstrated in a laboratory setting using commercially available anti-scatter grids and radiographic detectors. Open-source software was used to complete the CT reconstructions, including both parallel-beam and cone-beam options. Post-processing was developed to mitigate errors related to system stability over the long exposure times necessary for a CT acquisition. CT measurements of the scatter step wedge showed the ability to reconstruct all 3 modes (absorption, phase, and scatter), and revealed an uneven distribution of ZnO microspheres. CT measurements of plastic cylinders of different composition demonstrate the ability to recover quantitatively accurate absorption, phase, and scatter information.

Matching GBX-CT system geometry to the geometry of existing security screening CT systems having a large imaging region and short source-detector distance. For example, for reconstruction, adaptations to existing algorithms may be used to account for the gradient in phase and scatter sensitivity across the imaged volume, as well as for reconstructing scatter signal which can be orientation dependent. Also, while commercial anti-scatter gratings can be used and will result in low sensitivity, such systems could be viable if systematic effects can be identified and accounted for. Manufacturing finer gratings at the desired thicknesses may be possible using microfabrication. However, mechanical stability can be problematic, because any motion on the order of a fraction of a grating period can cause severe image artifacts. For commercial anti-scatter gratings, stability on the order of 0.001-0.002″ may be necessary, and for microfabricated grids this would be proportionally smaller. Use of a stationary gantry (with multiple non-rotating source-detector pairs) rather than rotating gantry may be more favorable for achieving this level of stability.

GBX system design can also be affected by available detector technology. For example, most GBX detection approaches will require that detector arrays be 2-D, and large areas may be favorable for increasing effective measurement time (or decreasing actual measurement time). Direct imaging options become available if pixel sizes are small, which may decrease complexity, but at fixed area this increases the number of pixels to be read out and processed per unit time. Both larger areas and smaller pixels may increase system costs. However, the added detection capabilities may outweigh the cost increase. In some examples, GBX-CT systems can include large field-of-view geometries, with detection performance being measured as a function of exposure or spatial resolution. Low-sensitivity configurations may also be suitable where systematic errors can be minimized. Higher sensitivity designs can leverage custom gratings and related fabrication methods as well as high stability mechanical configurations.

It should be noted that the benefits of additional signatures from GBX may still be leveraged by other system examples. GBX-CT becomes less difficult if the imaged volume is reduced. This reduces the sensitivity gradient across the imaged region, and for the same source-detector distance increases the geometric sensitivity. At the same time, if energy can be reduced due to smaller object sizes, sensitivity to both phase and scatter increases at lower x-ray energies. For example, portable or small object detection systems are possible. Another consideration is coupling data from a primary-screening CT instrument with GBX at a single view. Algorithms can be used that correlate single view projection data with a 3-D dual energy set, allowing for GBX features from a single projection to be mapped to a 3-D feature region in a CT dataset, leveraging the 3-D geometry to re-interpret the projection. This would involve registration between the two datasets but potentially offers a lower-cost route to incorporating GBX data.

In view of the many possible embodiments to which the principles of the disclosed technology may be applied, it should be recognized that the illustrated embodiments are only representative examples and should not be taken as limiting the scope of the disclosure. Alternatives specifically addressed in these sections are merely exemplary and do not constitute all possible alternatives to the embodiments described herein. For instance, various components of systems described herein may be combined in function and use. We therefore claim all that comes within the scope of the appended claims.

What is claimed is:

1. A system, comprising:
an x-ray optical arrangement that includes an x-ray source configured to emit an x-ray beam along a beam path through at least a portion of an object placement location situated to receive an object for inspection, through an object grating situated along the beam path, and to a detector situated along the beam path to receive the x-ray beam after propagating through the object grating and object placement location and to detect image data, wherein the object grating includes object grating elements arranged in an object grating pattern; and
at least one movement stage coupled to the object placement location and/or the x-ray optical arrangement and configured to provide rotational movement between the x-ray optical arrangement and the object placement location to provide a plurality of computed tomography imaging positions for the object by the x-ray optical arrangement;
wherein the object grating pattern and a positioning of the x-ray source, object grating, and detector in relation to each other are configured to provide a plurality of computed tomography detection modes in the image data.

2. The system of claim 1, wherein the plurality of detection modes includes at least two of absorption, scatter, or differential phase.

3. The system of claim 1, wherein the object grating elements include parallel line elements.

4. The system of claim 3, wherein the object grating is situated such that the parallel line elements of the object grating are parallel to a rotational axis of the movement stage.

5. The system of claim 4, wherein the at least one movement stage also provides for translational motion along a line parallel to the rotational axis between the x-ray optical arrangement and the object placement location, wherein the X-ray beam is configured with a height along the rotational axis that is less than a height of the object placement location, and wherein an effective detection height of the detector is less than the height of the object placement location, and wherein a plurality of computed tomography imaging positions comprise position taken at a plurality of different translational positions of the at least one movement stage.

6. The system of claim 5, wherein the effective detection height of the detector includes a sufficient number of pixels to effectively detect at least one entire fringe spacing.

7. The system of claim 3, further comprising a detector grating situated closer to the detector than the object placement location and including detector grating elements arranged in a detector grating pattern, wherein the detector grating elements include parallel line elements and the detector grating is situated such that the parallel line elements of the detector grating are perpendicular to the parallel line elements of the object grating.

8. The system of claim 7, wherein the detector grating pattern is configured in relation to the object grating pattern to allow for a beam hardening correction to the image data through a separation of pattern visibility loss due to scatter from pattern visibility loss due to spectral changes.

9. The system of claim 3, further comprising a source grating situated adjacent to the x-ray source and including a source grating pattern configured to produce a plurality of line sources from the x-ray beam.

10. The system of claim 9, wherein the source grating pattern includes a plurality of parallel line elements, and the source grating is situated such that the parallel line elements of the source grating are parallel to the parallel line elements of the object grating.

11. The system of claim 1, wherein the object grating is situated farther from the detector than the object placement location, or, wherein the object grating is situated closer to the detector than the object placement location.

12. The system of claim 1, wherein the object grating is situated less than 0.45L, 0.4L, 0.3L, or 0.2L distance from the x-ray source, where L is the distance between the x-ray source and the detector.

13. The system of claim 1, further comprising a processor and memory configured with processor executable instructions which cause the processor to apply a beam hardening correction to the image data through a separation of pattern visibility loss due to scatter from pattern visibility loss due to spectral changes.

14. The system of claim 2, further comprising a processor and memory configured with processor executable instructions which cause the processor to perform a computed tomography reconstruction with the image data for one or more of the computed tomography detection modes.

15. The system of claim 14, wherein the memory is configured with processor executable instructions which cause the processor to perform the computed tomography reconstruction with the image data for the differential phase computed tomography detection mode, including Fourier transforming sinograms of the image data, filtering the transformations using a signum filter, and backprojecting the filtered transformations through the object space to obtain a slice;
wherein the object grating includes parallel line elements arranged parallel to a rotational axis of the movement stage.

16. The system of claim 14, wherein the memory is configured with processor executable instructions which cause the processor to perform the computed tomography reconstruction with the image data for the differential phase computed tomography detection mode, including integrating differential phase image data, Fourier transforming sinograms of the differential phase image data, filtering the transformations using a ramp filter, and backprojecting the filtered transformations through the object space to obtain a slice.

17. The system of claim 1, further comprising a processor and memory configured with processor executable instructions which cause the processor to:
Fourier transform the detected image data of an object-free region from which pre- and post-exposure grating-only image data can be compared; and
fit Fourier components of the transformed detected image data that are in a Fourier space region around a source grid oscillation peak, to a linear combination of Fourier components of the pre- and post-exposure grating-only image data so that the linear combination can be used as a reference grating image during image processing to extract the phase and scatter images.

18. A method, comprising:
with an x-ray optical arrangement having an x-ray source configured to emit an x-ray beam along a beam path through at least a portion of an object placement location situated to receive an object for inspection, through an object grating situated along the beam path that includes object grating elements arranged in an object grating pattern, and to a detector situated along the beam path to receive the x-ray beam after propagating through the object grating and the object placement location to produce image data, and
with at least one movement stage coupled to the object placement location and/or the x-ray optical arrangement and configured to provide rotational movement between the x-ray optical arrangement and the object placement location to provide a plurality of computed tomography imaging positions for the object by the x-ray optical arrangement:
directing the x-ray beam through a first computed tomography imaging position and collecting first image data with the detector;
rotating the object placement location and/or x-ray optical arrangement with the movement stage to a second computed tomography imaging position; and
directing the x-ray beam through the second computed tomography imaging position and collecting second image data with the detector;
wherein the object grating pattern and a positioning of the x-ray source, object grating, and detector in relation to each other are configured to provide a plurality of computed tomography detection modes in each of the first image data and second image data.

19. The method of claim 18, wherein the plurality of detection modes includes absorption, scatter, and/or differential phase.

20. The method of claim 18, wherein the object grating elements include parallel line elements.

21. The method of claim 20, wherein the object grating is situated such that the parallel line elements of the object grating are parallel to a rotational axis of the movement stage.

22. The method of claim 21, wherein the at least one movement stage also provides for translational motion along a line parallel to the rotational axis between the x-ray optical arrangement and the object placement location, wherein the X-ray beam is configured with a height along the rotational axis that is less than a height of the object placement location, and wherein an effective detection height of the detector is less than the height of the object placement location, and wherein a plurality of computed tomography imaging positions comprise position taken at a plurality of different translational positions of the at least one movement stage.

23. The method of claim 22, wherein the effective detection height of the detector includes a sufficient number of pixels to effectively detect at least one entire fringe spacing.

24. The method of claim 20, wherein the optical arrangement comprises a detector grating situated adjacent to the detector and including detector grating elements arranged in a detector grating pattern, wherein the detector grating elements include parallel line elements and the detector grating is situated such that the parallel line elements of the detector grating are perpendicular to the parallel line elements of the object grating.

25. The method of claim 24, wherein the detector grating pattern is configured in relation to the object grating pattern to allow for a beam hardening correction to the image data through a separation of pattern visibility loss due to scatter from pattern visibility loss due to spectral changes.

26. The method of claim 20, further comprising a source grating situated adjacent to the x-ray source and including a source grating pattern configured to produce a plurality of line sources from the x-ray beam.

27. The method of claim 26, wherein the source grating pattern includes a plurality of parallel line elements, and the source grating is situated such that the parallel line elements of the source grating are parallel to the parallel line elements of the object grating.

28. The method of claim 18, wherein the object grating is situated farther from the detector than the object placement location, or, wherein the object grating is situated closer to the detector than the object placement location.

29. The method of claim 18, wherein the object grating is situated less than 0.45L, 0.4L, 0.3L, or 0.2L distance from the x-ray source, where L is the distance between the x-ray source and the detector.

30. The method of claim 18, further comprising, with a processor and memory configured with processor executable instructions, applying a beam hardening correction to the image data through a separation of pattern visibility loss due to scatter from pattern visibility loss due to spectral changes.

31. The method of claim 19, further comprising, with a processor and memory configured with processor executable instructions, performing a computed tomography reconstruction with the image data for one or more of the computed tomography detection modes.

32. The method of claim 31, wherein the performing includes performing the computed tomography reconstruction with the image data for the differential phase computed tomography detection mode, including by Fourier transforming sinograms of the image data, filtering the transformations using a signum filter, and backprojecting the filtered transformations through the object space to obtain a slice;
wherein the object grating includes parallel line elements arranged parallel to a rotational axis of the movement stage.

33. The method of claim 31, wherein the performing includes performing the computed tomography reconstruction with the image data for the differential phase computed tomography detection modes, including by integrating differential phase image data, Fourier transforming sinograms of the differential phase image data, filtering the transformations using a ramp filter, and backprojecting the filtered transformations through the object space to obtain a slice.

34. The method of claim 18, further comprising, a processor and memory configured with processor executable instructions which provide for:
Fourier transforming the detected image data of an object-free region from which pre- and post-exposure grating-only image data can be compared; and
fitting Fourier components of the transformed detected image data that are in a Fourier space region around a source grid oscillation peak, to a linear combination of Fourier components of the pre- and post-exposure grating-only image data so that the linear combination can be used as a reference grating image during image processing to extract the phase and scatter images.

35. A non-transitory computer readable storage medium storing one or more programs, the one or more programs comprising instructions, that are executable by one or more processors operably connected to an x-ray imaging system that comprises:
an x-ray optical arrangement that includes an x-ray source configured to emit an x-ray beam along a beam path through at least a portion of an object placement location situated to receive an object for inspection, through an object grating situated along the beam path, and to a detector situated along the beam path to receive the x-ray beam after propagating through the object grating and object placement location and to detect image data, wherein the object grating includes object grating elements arranged in an object grating pattern; and
at least one movement stage coupled to the object placement location and/or the x-ray optical arrangement and configured to provide rotational movement between the x-ray optical arrangement and the object placement location to provide a plurality of computed tomography imaging positions for the object by the x-ray optical arrangement;
wherein the object grating pattern and a positioning of the x-ray source, object grating, and detector in relation to each other are configured to provide a plurality of computed tomography detection modes in the image data;
wherein the instructions cause the x-ray imaging system to collect the image data at one or more of the computed tomography imaging positions.
